(19) United States
(12) Patent Application Publication
(10) Pub. No.: US 2009/0123940 A1
(43) Pub. Date: May 14, 2009

(54) MOLECULES INVOLVED IN SYNAPTOGENESIS AND USES THEREFOR

(75) Inventors: Paul F. Worley, Baltimore, MD (US); Desheng Xu, Towson, MD (US); Richard William Cho, Newton Center, MA (US); Radhika Chinthamani Reddy, Portland, OR (US)

Correspondence Address: DLA PIPER LLP (US), 4365 EXECUTIVE DRIVE, SUITE 1100, SAN DIEGO, CA, 92121-2133, US

(73) Assignee: THE JOHNS HOPKINS UNIVERSITY, Baltimore, MD (US)

(21) Appl. No.: 12/249,850

(22) Filed: Oct. 10, 2008

Related U.S. Application Data
(60) Provisional application No. 60/979,609, filed on Oct. 12, 2007.

(51) Int. Cl. G01N 33/53; C07K 2/00; C07H 21/00; C07K 16/28

(52) U.S. Cl. 435/7.2; 530/300; 536/23.1; 530/387.1

(57) ABSTRACT

The present invention is based on the discovery that neuronal pentraxins play a role in the clustering and internalization of AMPA receptors, synaptogenesis, and metabotropic glutamate receptor-mediated long term depression (LTD) of a synapse. Accordingly, there are provided methods of identifying compounds that that modulate mGluR-mediated AMPA receptor internalization and LTD. Further provided are cleavage products of a member of the neuronal pentraxin family, neuronal pentraxin receptor (NPR). Also provided are isolated peptides comprising the Narp association regions 1 and 2 (NAR1 and NAR2, respectively) and the Narp binding motif (NBM) of AMPA receptors. Finally, there are provided antibodies that block binding of neuronal pentraxins to AMPA receptors, in particular, antibodies that bind NAR1, or NAR2, or NBM.

### RELATED APPLICATIONS

This application claims the benefit of priority under 35 U.S.C. § 119(e) to U.S. Provisional Application Ser. No. 60/979,609, filed Oct. 12, 2007, the entire content of which is incorporated herein by reference.

### GRANT INFORMATION

This invention was made with Government support under Grant Nos. K08NS01652, F31-AG19581-02, P50-MH068830, R01NS36715, R01NS39156, R01MH53608, and K02 MH01152, awarded by the National Institutes of Health. The government has certain rights in the invention.

### FIELD OF THE INVENTION

The present invention relates generally to molecules involved in synaptogenesis, and more specifically to the role of neuronal pentraxins in the formation of synapses and the clustering of receptors in synapses.

### BACKGROUND OF THE INVENTION

The mature central nervous system exhibits the capacity to alter cellular interactions as a function of the activity of specific neuronal circuits. This capacity is believed to underlie learning and memory storage, age-related memory loss, tolerance to and dependence on drugs of abuse, recovery from brain injury, epilepsy as well as aspects of postnatal development of the brain (Schatz, C., Neuron, 5:745, 1990). Currently, the role of activity-dependent synaptic plasticity is best understood in the context of learning and memory. Cellular mechanisms underlying activity-dependent plasticity are known to be initiated by rapid, transmitter-induced changes in membrane conductance properties and activation of intracellular signaling pathways (Bliss and Collingridge, Nature, 361:31, 1993). Several lines of evidence also indicate a role for rapid synthesis of mRNA and protein in long-term neuroplasticity. For example, classical studies of learning and memory demonstrate a requirement for protein synthesis in a long-term, but not short-term memory (Flexner, et al., Science, 141:57, 1963; Agranoff, B., Basic Neurochemistry, 3rd Edition, 1981; Davis and Squire, Physiol. Bull., 96:518, 1984), and long-term enhancement of synaptic connectivity, studied in cultured invertebrate neurons (Montarolo, et al., Science, 234:1249, 1986; Bailey, et al., Neuron, 9:749, 1992) or in the rodent hippocampus (Frey, et al., Science, 260:1661, 1993; Nguyen, et al., Science, 265:1004, 1194), is blocked by inhibitors of either RNA or protein synthesis. Importantly, inhibitors of macromolecular synthesis are most effective when administered during a brief time window surrounding the conditioning stimulus indicating a special requirement for molecules that are rapidly induced (Goelet, et al., Nature, 322:419, 1986).

Immediate early genes (IEGs) are rapidly induced in neurons by neurotransmitter stimulation and synaptic activity and are hypothesized to be part of the macromolecular response required for long-term plasticity (Goelet, et al., supra; Sheng and Greenberg, Neuron, 4:477, 1990; Silva and Giese, Neurobiology, 4:413, 1994). To identify cellular mechanisms that may contribute to long-term plasticity in the vertebrate brain, differential cloning techniques have been used to identify genes that are rapidly induced by depolarizing stimuli (Nedivi, et al., Nature, 363:713, 1993; Qian, et al., Nature, 361:453, 1993; Yamagata, et al., Neuron, 11:371, 1993; Yamagata, et al., Learning and Memory 1:140, 1994; Yamagata, et al., Journal of Biological Chemistry, 269:16333, 1994; Andreasson and Worley, Neuroscience, 69:781, 1995; Lyford, et al., Neuron, 14:433, 1995). In contrast to the earlier focus on transcription factors, many of the newly characterized IEGs represent molecules that can directly modify the function of cells and include growth factors (Nedivi, et al., supra; Andreasson and Worley, supra), secreted enzymes that can modify the extracellular matrix, such as tissue plasminogen activator (Qian, et al., supra), enzymes involved in intracellular signaling, such as prostaglandin synthase (Yamagata, et al., supra), and a novel homolog of H-Ras, termed Rheb (Yamagata, et al., supra), as well as a novel cytoskeleton-associated protein, termed Arc (Lyford, et al., supra). The remarkable functional diversity of this set of rapid response genes is representative of the repertoire of cellular mechanisms that are likely to contribute to activity-dependent neuronal plasticity.

The identification of molecules regulating the aggregation of neurotransmitter receptors at synapses is central to understanding the mechanisms of neural development, synaptic plasticity and learning. The most well characterized model for the synaptic aggregation of ionotropic receptors is the neuromuscular junction. Early work showed that contact between the axon of a motor neuron and the surface of a myotube rapidly triggers the accumulation of preexisting surface acetylcholine receptors (Anderson and Cohen, J. Physiol. 268:757-773, 1977; Frank and Fischbach, J. Cell. Biol. 83:143-158, 1979). Subsequent work has shown that agrin, a complex glycoprotein secreted by the presynaptic terminal, activates a postsynaptic signal transduction cascade (reviewed by Colledge and Froehner, Curr. Opin. Neurobiol. 8:357-63, 1998), that leads to receptor clustering by the membrane associated protein rapsyn.

In the central nervous system, ionotropic glutamate receptors are the major excitatory neurotransmitter receptors and are divided into three broad classes, termed AMPA, NMDA and kainate type receptors, on the basis of molecular and pharmacological criteria (Hollmann, M., and Heinemann, S., Ann. Rev. Neurosci. 17:31-108, 1994). The predominant charge carrier during routine fast excitatory synaptic transmission is the AMPA type receptor, while NMDA receptors contribute a significant calcium current, which is thought to modulate signal transduction pathways. Functional AMPA receptors are multimeric complexes of the homologous subunits GluR1-4 (Rosenmund et al., Science 280:1596-9, 1998; Mano and Teichberg, Neuroreport 9: 327-31 1998) which share about 60% to 70% homology at the amino acid level (Keinanen et al., Science 249:556-60, 1990). A variety of studies have shown that glutamate receptors are highly concentrated in neurons at excitatory synapses on dendritic spines and shafts.

Significant advances in the identification of molecules involved in excitatory synapse formation have recently occurred using genetic and biochemical techniques. A family of cytoplasmic proteins containing protein-protein interaction motifs, called PDZ domains, have been implicated in the clustering of both NMDA and AMPA receptors at synapses (O'Brien et al., Curr. Opin. Neurobiol. 8:364-9 1998). These PDZ domain containing proteins are thought to intracellularly cross link receptors and couple them to the cytoskeleton. The PSD-95 family of proteins contain three PDZ domains, which directly interact with the C-termini of NMDA receptor subunits and may be important in NMDA receptor clustering (Kornau et al., Science 22:1737-40, 1995). Similarly, the neuronal proteins GRIP (Dong et al., Nature 386:279-84, 1997), ABP (Srivastava et al., Neuron 21:581-91, 1998), and Pick1 (Xia et al., Neuron 22:179-187, 1998), each of which contains one or more PDZ domains, interact with the C-terminus of AMPA receptors and may be important in receptor targeting (Dong et al., supra). The extracellular factors that facilitate the formation of excitatory synapses in the central nervous system have not been identified.

An additional level of complexity in the formation of central excitatory synapses stems from the fact that two populations of neurons exist (termed spiny and aspiny) which receive excitatory input in mutually exclusive patterns (Sloper and Powell, 1979; Harris and Kater, 1994). Spiny neurons, such as hippocampal pyramidal neurons receive more than 90% of their excitatory input onto dendritic spines, while shaft synapses on these neurons are largely inhibitory. Aspiny neurons such as hippocampal interneurons and most spinal neurons receive both excitatory and inhibitory synapses on their dendritic shafts. Emerging evidence indicates that excitatory synapses on spines and shafts have different structural and functional properties which may imply different molecular mechanisms in their formation and maintenance (O'Brien et al., J. Neurosci. 17:7339-50, 1997; Rao et al., J. Neurosci. 18:1217-29, 1998, amongst others).

### SUMMARY OF THE INVENTION

Narp was originally identified using a subtractive cloning strategy from stimulated hippocampus (see U.S. Pat. No. 5,767,252, incorporated by reference). The present invention shows that Narp and other neuronal pentraxins play a role in clustering of AMPA receptors. It therefore follows that other members of the long pentraxin family may play a role in synaptognesis. Thus, the present invention is directed to methods and compositions employing long pentraxins in general, and the neuronal pentraxins in particular. Neuronal activity regulated pentraxin (Narp) and neuronal pentraxin receptor (NPR) are discussed for purposes of exemplary long pentraxins of the invention. It should be understood that the methods of the invention are useful for all long pentraxins, which are known in the art as a family of proteins that are found in the brain and share a core pentraxin domain. The neuronal pentraxins (NPs), are long pentraxins and include neuronal pentraxin 1 (NP1), Neuronal activity regulated pentraxin (Narp), and neuronal pentraxin receptor (NPR). NP1 and Narp are secreted Ca²⁺-dependent lectins that are present at excitatory synapses. However, NPR possesses an N-terminal transmembrane domain and a short (approximately 7 amino acid) intracellular sequence. The extracellular portion of NPR is highly homologous to Narp and NP1. Narp and NP-1 additionally include an extended N-terminus that appears to function in clustering. NP-1 is secreted and forms clusters on the surface of expressing cells, similar to observations with Narp.

In one aspect of the invention, a method is provided for identifying a compound which affects the formation of AMPA receptors into aggregates. The method includes incubating the compound and a pre-synaptic cell expressing Long pentraxin (e.g., Narp, NP1, NPR, or a functional fragment thereof) under conditions sufficient to allow the compound to interact with the cell, and determining the effect of the compound on the formation of AMPA receptors into aggregates in the pre-synaptic cell or in a post-synaptic cell synapsing with the pre-synaptic cell. The formation of AMPA receptors into aggregates of the pre-synaptic cell contacted with the compound, or in the post-synaptic cell is compared with the formation of AMPA receptors into aggregates in a pre-synaptic cell not contacted with the compound or a post-synaptic cell synapsing with the pre-synaptic cell not contacted with the compound. In certain embodiments, the long pentraxin is NPR or a functional fragment thereof and the pre-synaptic cell optionally expresses a metabotropic glutamate receptor (mGluR) and a tumor necrosis a converting enzyme (TACE).

A method is provided for identifying a compound which affects the formation of synaptic connections. The method includes incubating the compound and a cell expressing Long pentraxin (e.g., Narp, NP1, NPR, or a functional fragment thereof) under conditions sufficient to allow the compound to interact with the cell and determining the effect of the compound on the formation of synaptic connections. The synaptic connections of the cell contacted with the compound is compared with the synaptic connections of a cell not contacted with the compound. In certain embodiments, the long pentraxin is NPR or a functional fragment thereof and the pre-synaptic cell optionally expresses a metabotropic glutamate receptor (mGluR) and a tumor necrosis α converting enzyme (TACE).

A method is provided for identifying a compound that modulates immediate early gene expression. The method includes contacting a test compound with a sample containing a nucleic acid encoding long pentraxin (e.g., Narp, NP1, NPR, or a functional fragment thereof) and determining whether the test compound effects the expression of the immediate early gene nucleic acid, wherein the presence of an effect indicates that the test compound modulates immediate early gene expression.

A method is provided for increasing the number of excitory synapses of a neuron, including introducing into the neuron a polynucleotide sequence encoding a Long pentraxin (e.g., Narp, NP1, NPR, or a functional fragment thereof) operatively linked to a promoter, thereby increasing the number of excitory synapses of the neuron.

A method is provided for increasing the number of excitory synapses of a neuron, including introducing into the neuron a Long pentraxin (e.g., Narp, NP1, NPR, or a functional fragment thereof) polypeptide, thereby increasing the number of excitory synapses of the neuron.

A method is provided for treating a subject with a disorder associated with a decrease in a function or expression of Long pentraxin (e.g., Narp, NP1, NPR, or a functional fragment thereof), including administering to the subject a therapeutically effective amount of a compound that augments Long pentraxin (e.g., Narp, NP1, NPR, or a functional fragment thereof) function or expression.

A method is provided for treating a subject with a disorder associated with an increase in a function or expression of Long pentraxin (e.g., Narp, NP1, NPR, or a functional fragment thereof), including administering to the subject a therapeutically effective of a compound that inhibits Long pentraxin (e.g., Narp, NP1, NPR, or a functional fragment thereof) function or expression.

A method is provided for treating a patient having or at risk of having a disorder associated with decreased Long pentraxin (e.g., Narp, NP1, NPR, or a functional fragment thereof) expression. The method includes introducing into a cell of a patient having a disorder associated with decreased Long pentraxin (e.g., Narp, NP1, NPR, or a functional fragment thereof) expression or function, a polynucleotide sequence encoding a Long pentraxin polypeptide (e-g., Narp, NP1, NPR, or a functional fragment thereof) operatively linked to a promoter, thereby augmenting a function of Long pentraxin (e.g., Narp, NP1, NPR, or a functional fragment thereof).

A method is provided for treating a subject having a deficiency in a neuron's immediate early gene responsiveness to a stimulus. The method includes administering a nucleic acid encoding a Long pentraxin (e.g., Narp, NP1, NPR, or a functional fragment thereof) polypeptide to said subject, wherein the administration results in amelioration of the deficiency.

A method is provided for identifying a compound that modulates metabotropic glutamate receptor (mGluR) dependent internalization of AMPA receptors, wherein the method includes (a) incubating the compound and a post-synaptic cell expressing a neuronal pentraxin receptor (NPR) or a functional fragment thereof and an mGluR1/5 under conditions sufficient to allow the compound to interact with the cell; (b) determining the effect of the compound on the internalization of AMPA receptors upon stimulation with DHPG; and (c) comparing the mGluR-dependent internalization of AMPA receptors upon stimulation with DHPG in the presence of compound, to the mGluR-dependent internalization of AMPA receptors upon stimulation with DHPG in the absence of compound, wherein a difference in the mGluR-dependent internalization of AMPA receptors indicates that the compound modulates mGluR-dependent internalization of AMPA receptors.

A method is provided for identifying a compound that modulates metabotropic glutamate receptor (mGluR) dependent long term depression (LTD) of a synapse, wherein the method includes (a) incubating the compound and a post-synaptic cell expressing a neuronal pentraxin receptor (NPR) or a functional fragment thereof and an mGluR1/5 under conditions sufficient to allow the compound to interact with the cell; (b) determining the effect of the compound on the mGluR-dependent LTD upon stimulation with DHPG; and (c) comparing the mGluR-dependent LTD upon stimulation with DHPG in the presence of compound to the mGluR-dependent LTD upon stimulation with DHPG in the absence of compound, wherein a difference in the mGluR-dependent LTD indicates that the compound modulates mGluR-dependent LTD.

A method is provided for identifying compounds that inhibit clustering of AMPA receptors wherein the method includes, (a) contacting, in the presence and absence of a test compound: (i) a long pentraxin or fragment thereof capable of binding the Narp association region 1 (NAR1) region of AMPA receptors and (ii) a peptide containing an NAR1 region of AMPA receptors or functional variant thereof, wherein the peptide is capable of binding the long neural pentraxin, and (b) comparing the binding between the long pentraxin or fragment thereof and the peptide containing the NAR1 region in the presence and absence of the test compound, wherein a decrease in the amount of binding in the presence of the test compound indicates a compound that inhibits clustering of AMPA receptors.

A method is provided for identifying compounds that stimulate clustering of AMPA receptors including, (a) contacting, in the presence and absence of a test compound: (i) a long pentraxin or fragment thereof capable of binding the NAR1 region of AMPA receptors and (ii) a peptide containing an NAR1 region of AMPA receptors or functional variant thereof, wherein the peptide is capable of binding the long neural pentraxin, and (b) comparing the binding between the long pentraxin or fragment thereof and the peptide containing the NAR1 region in the presence and absence of the test compound, wherein an increase in the amount of binding in the presence of the test compound indicates a compound that stimulates clustering of AMPA receptors.

Also provided are isolated fragments of NPR, particularly NPR fragments that are biologically functional. In certain embodiments, the fragment is capable of internalizing AMPA receptors, clustering AMPA receptors, or modulating metabotropic glutamate receptor (mGluR) dependent long term depression (LTD) of a synapse. In some embodiments, fragments include those produced by cleavage of NPR at cleavage site A or cleavage site B. In one aspect the fragment includes amino acid residues 1-175 of NPR; amino acid residues 36-494 of NPR; or amino acid residues 176-494 of NPR. Also provided are variants of the recited fragments.

Also provided are polynucleotides encoding NPR fragments. In certain embodiments, the polynucleotides encode the fragment containing amino acid residues 1-175 of NPR, or the fragment containing amino acid residues 36-494 of NPR, or the fragment containing amino acid residues 176-494 of NPR. Also provided are polynucleotides encoding variant fragments.

Also provided herein are fragments of AMPA receptors capable of binding a long pentraxin or a cadherin protein. In certain embodiments, the fragment is a NAR1 peptide. In one aspect, the NAR1 peptide contains the following sequence VDWKRPKYTSALTYDGVKVMAEAFQSLRRQRIDISRRGNAGDC (SEQ ID NO:11). In other embodiments, a Narp binding motif (NBM) of about 12 amino acids within a NAR1 peptide is sufficient to bind long pentraxins. In certain embodiments, the fragment has the sequence LRRQRIDISRRG (SEQ ID NO:24).

In other embodiments, the fragment is a NAR2 peptide. In certain embodiments, the fragment is a NAR2 peptide. In one aspect, the NAR2 peptide contains the following sequence SVFVRTTEEGMIRVRKSKGKYAYLLESTMNEYIEQRKPCDTMKVG (SEQ ID NO:25).

Also provided herein are nucleic acids encoding a NAR1 peptide, an NBM peptide or a NAR2 peptide.

Further provided is an antibody that blocks binding of a long pentraxin to AMPA receptors. In certain embodiments, the antibody is against the NAR1 region of AMPA receptors.

A pharmaceutical composition is provided including a therapeutically effective mount of an expression vector including a nucleic acid encoding a long pentraxin (e.g., Narp, NP1, NPR, or a functional fragment thereof) polypeptide or a conservative variant thereof and a pharmaceutically acceptable carrier.

Also provided is a pharmaceutical composition including a therapeutically effective mount of a long pentraxin (e.g., Narp, NP1, NPR, or a functional fragment thereof) polypeptide or a conservative variant thereof and a pharmaceutically acceptable carrier.

Further provided are antibodies that block binding of a neuronal pentraxin to the AMPA receptor (AMPAR). In certain embodiments, the antibody binds the NAR1 region of AMPAR.

### BRIEF DESCRIPTION OF THE DRAWINGS

FIGS. 1A and 1B show the nucleotide sequence of Narp cDNA (SEQ ID NO: 1) and its predicted amino acid sequence (SEQ ID NO:2). The putative signal peptide of 16 amino acids is underlined. A dot indicates the predicted first amino acid of the mature protein. Putative glycosylation sites are circled. Two putative ATTTA mRNA instability motifs are present in the 3′ untranslated region and are boxed. The putative polyadenylation signal (ATTAAA) is underlined (sequence as shown in: Tsui et al., J. Neurosci. 16:2463-78, 1996, herein incorporated by reference.

FIG. 2 shows a schematic of the NPR protein. The top line shows a full-length NPR, including the 2 coiled-coil domains (“1st Coil” and “2nd Coil”) and the C-terminal pentraxin domain. Cysteines predicted to form disulfide linkages with other NPR subunits as well as other NPs are labeled. Regions of NPR used to generate Ab LFC-NPR, Ab 4999, and Ab 4450 are identified. Cleavage Sites A and B are identified and occur just before L36 and D176, respectively. Cleavage produces three NPR fragments, Long Form Cleaved (“LFC-NPR,” middle line), N-terminal Fragment (“NTF-NPR,” bottom line), and C-terminal Fragment (“CTF-NPR,” bottom line).

FIG. 3 shows schematics of Narp (solid bars), NPR (striped bars), and the chimeric proteins, CM-1 through CM-6, constructed from Narp and NPR.

FIG. 4A shows a plot of the Western blot band intensities corresponding to LFC-NPR in primary cortical culture lysates after 5 min of treatment mGluR1/5 agonist DHPG. FIG. 4B shows a plot of the Western blot band intensities corresponding to surface GluR1 in primary cortical cultures. FIG. 4C shows a plot of the Western blot band intensities corresponding to surface LFC-NPR from primary cortical cultures. FIG. 4D shows a plot of the Western blot band intensities corresponding to surface GluR1 from control and NP TKO (neuronal pentraxin total knockout mouse) primary cortical neurons. FIG. 4E shows a plot representing the quantification of surface GluR1 puncta density after vehicle control or DHPG stimulation with or with or without TAPI-2. FIG. 4F shows a plot representing the quantification of density of surface GluR1 per unit length after vehicle control or DHPG stimulation cultured neurons from control and NP TKO mice. FIG. 4G shows a plot representing the quantification of internalized GluR1 density per unit length after vehicle control or DHPG stimulation in cultured neurons from control and NP TKO mice.

FIG. 5A shows a plot of DHPG induced mGluR1/5-dependent LTD (expressed as percent baseline fEPSP) in hippocampal slices derived from control mice and upon bath application of TAPI-2 during the baseline and acute DHPG. FIG. 5B shows a plot of DHPG induced mGluR1/5-dependent LTD in slices from NPR KO and NP TKO mice.

FIG. 5C shows PP-1 Hz induced mGluR1/5-dependent LTD in slices from control mouse with or without application of TAPI-2, and in slices from NP TKO mouse.

FIG. 6A shows plots of LTD experiments in Purkinje cells derived from mice harboring mutations in NP genes: control; NP TKO; NPR KO; and NPR KO, NPR wt rescue. Representative current traces are shown (inset). FIG. 6B shows plots of LTD experiments in Purkinje cells treated with TACE inhibitors: TAPI-2; GM6001; GM6001 control compound.

FIG. 7A shows graphs depicting the effect of TAPI-2 application on presynaptic function before LTD induction by DHPG or PP-1 Hz as indexed by paired pulse ratio (PPR).

FIG. 7B shows graphs depicting presynaptic function in NPR KO and NP TKO compared to control as indexed by PPR. FIG. 7C shows graphs depicting basal synaptic strength in NPR KO and NP TKO compared to control as indexed by analysis of input/output relation. The input/output relation between the fiber volley and the fEPSP slope was determined over the range of stimulus intensities indicated.

FIG. 8 shows a schematic depicting a proposed model of NP function at excitatory synapses.

FIG. 9A shows an illustration depicting the receptor topology and domain structure of GluR1. FIG. 9B shows GluR1 and GluR6 constructs.

FIG. 10A shows an illustration of the domain structure of GluR1. FIG. 10B shows an alignment of NAR1 for GluR1-4 and 6 and an alignment of NAR2 for GluR1-4 and 6.

FIG. 11 shows an alignment of NAR1 sequences from GluR1 from several species. The Narp binding motif (NBM) is underlined.

FIG. 12 shows a plot of the diffusion coefficients of several GluR1 constructs in the presence or absence of Narp.

FIGS. 13A and B show plots of the co-localization of HA GluR1 or HA GluR6 constructs with GFP-PSD-95.

FIGS. 14A-C show amino acid sequences of NPR fragments: amino acid residues 1-175 of NPR (SEQ ID NO:5); amino acid residues 36-494 of NPR (SEQ ID NO:7); and amino acid residues 176-494 of NPR (SEQ ID NO:7).

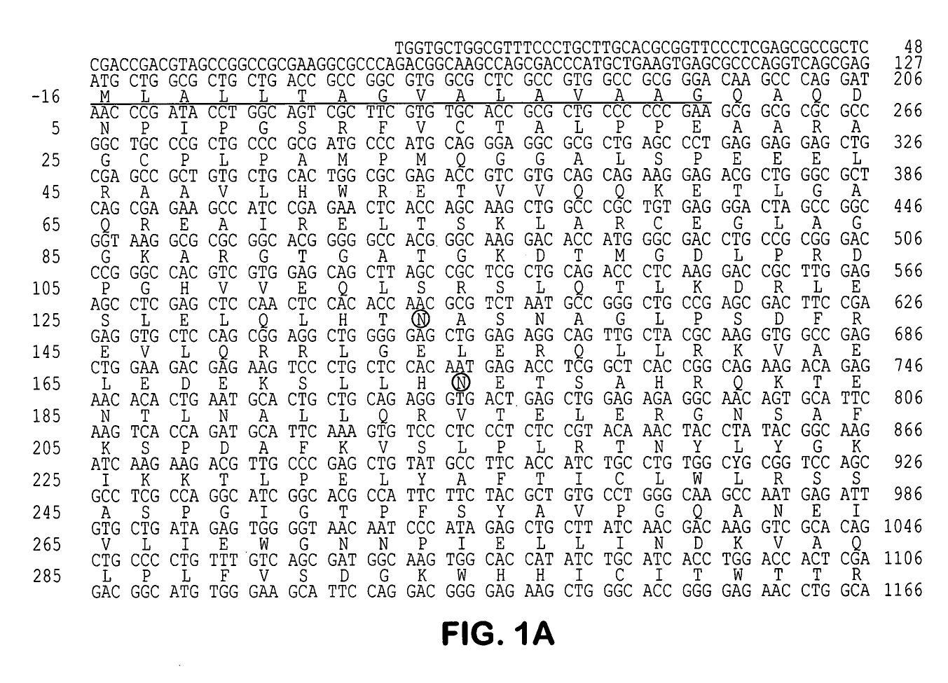

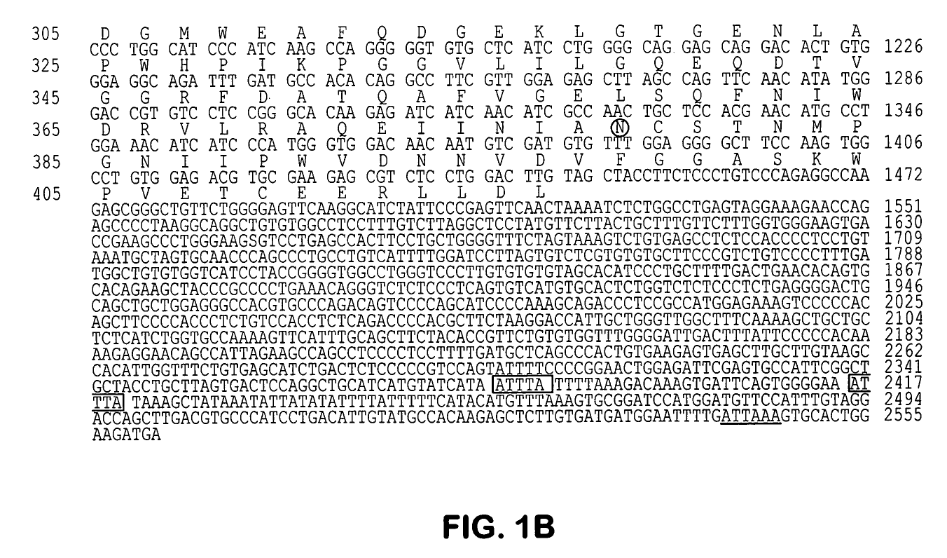

### DETAILED DESCRIPTION OF THE INVENTION

The present invention is based on the discovery that neuronal pentraxins play a role in the clustering and internalization of AMPA receptors, synaptogenesis, and metabotropic glutamate receptor-mediated long term depression (LTD) of a synapse. Accordingly, there are provided methods of identifying compounds that modulate AMPA receptor clustering and synaptogenesis. Further provided are methods of identifying compounds that modulate mGluR-mediated AMPA receptor internalization and LTD. Further provided are methods of treatment of disease states resulting from or characterized by an increase or decrease in function or expression of neuronal pentraxins. Also provided are pharmaceutical compositions including neuronal pentraxin polypeptide or nucleic acids encoding neuronal pentraxins.

As used herein the term “neuronal pentraxins” refers to a group of long pentraxin that include, but are not limited to, neuronal pentraxin 1 (NP1), neuronal pentraxin 2 (Narp), and neuronal pentraxin receptor (NPR).

Narp was originally identified using a subtractive cloning strategy from stimulated hippocampus (U.S. Pat. No. 5,767,252; Tsui et al., J. Neurosci. 16: 2463-78, 1996, both of which are herein incorporated by reference) and was identified as a member of the newly recognized subfamily of “long pentraxins” that includes neuronal pentraxin 1 (NP1) and 2 (Narp), which are found in the brain (Schlimgen et al., Neuron 14:519-26, 1995), TSG-14 (Lee et al., J. Immunol. 150:1804-12, 1993); a TNF inducible acute phase reactant; and Apexin (Reid and Blobel, J. Biol. Chem. 269:32615-20, 1994; Rothstein et al., Neuron 13:713-25, 1994), which is localized to the acrosome of mature sperm. These molecules are similar in structure in that they possess a C-terminal pentraxin domain and a ˜200 aa unique N-terminus whose function is unknown (Goodman et al., Cytokine Growth Factor Rev. 7:191-202, 1996). The pentraxin domain on Narp is similar to the mammalian proteins C-reactive protein (CRP), serum amyloid protein (SAP), as well as highly conserved homologs from species as distant as Limulus. (Tsui et al., 1996, supra). Pentraxins are secreted proteins that self multimerize to form pentamers and may further dimerize to form decamers, (Gewurz et al., Curr. Opin. Immunol. 7:54-64, 1995). A crystal structure of SAP showed that the pentraxin sugar binding motif is remarkably homologous in secondary and tertiary structure to the plant lectin concanavalin A (Emsley et al., Nature 367, 338-45, 1994), a feature that is conserved in Narp (Tsui et al., 1996, supra). The physiological roles of pentraxins have remained obscure, although CRP has been postulated to play a role in non-antibody mediated immune responses by binding and aggregating bacteria and other pathogens (Siegel et al., J. Exp. Med. 140:631-47, 1974; Siegel et al., J. Exp. Med. 142: 709-21 1975).

An exemplary polynucleotide encoding Narp is set forth as SEQ ID NO: 1. The term “polynucleotide” or “nucleic acid sequence” refers to a polymeric form of nucleotides at least 10 bases in length. By “isolated polynucleotide” is meant a polynucleotide that is not immediately contiguous with both of the coding sequences with which it is immediately contiguous (one on the 5′ end and one on the 3′ end) in the naturally occurring genome of the organism from which it is derived. The term therefore includes, for example, a recombinant DNA which is incorporated into a vector; into an autonomously replicating plasmid or virus; or into the genomic DNA of a prokaryote or eukaryote, or which exists as a separate molecule (e.g., a cDNA) independent of other sequences. The nucleotides of the invention can be ribonucleotides, deoxyribonucleotides, or modified forms of either nucleotide. A polynucleotide encoding Narp includes “degenerate variants,” sequences that are degenerate as a result of the genetic code. There are 20 natural amino acids, most of which are specified by more than one codon. Therefore, all degenerate nucleotide sequences are included in the invention as long as the amino acid sequence of a polypeptide encoded by the nucleotide sequence of SEQ ID NO: 1 is functionally unchanged.

A polynucleotide encoding Narp includes a polynucleotide encoding functional Narp polypeptides as well as functional fragments thereof. As used herein, the term “functional polypeptide” refers to a polypeptide which possesses biological function or activity which is identified through a defined functional assay and which is associated with a particular biologic, morphologic, or phenotypic alteration in the cell. The term “functional fragments of Narp polypeptide,” refers to all fragments of a Narp that retain a Narp activity, e.g., the ability to recruit AMPA receptors into aggregates or to facilitate the formation of new excitatory synapses. Biologically functional fragments, for example, can vary in size from a polypeptide fragment as small as an epitope capable of binding an antibody molecule to a large polypeptide capable of participating in the characteristic induction or programming of phenotypic changes within a cell.

A functional Narp polypeptide includes the polypeptide as set forth in SEQ ID NO:2 and conservative variations thereof.

The term “conservative variation” as used herein denotes the replacement of an amino acid residue by another, biologically similar residue. Examples of conservative variations include the substitution of one hydrophobic residue such as isoleucine, valine, leucine or methionine for another, or the substitution of one polar residue for another, such as the substitution of arginine for lysine, glutamic for aspartic acid, or glutamine for asparagine, and the like. The term “conservative variation” also includes the use of a substituted amino acid in place of an unsubstituted parent amino acid provided that antibodies raised to the substituted polypeptide also immunoreact with the unsubstituted polypeptide.

A second member of the neuronal pentraxin family is neuronal pentraxin 1 (NP1), which was identified in the rat as a binding protein for the snake venom toxin taipoxin (Schlimgen et al., Neuron 14:519-26, 1995). The human NP1 homolog was cloned by screening a human cerebellar cDNA library with the rat NP1 gene as a probe (Omeis et al., Genomics 36(3):543-5, 1996). The gene, designated NPTX1, encodes a predicted 430-amino acid protein that is 95% identical to rat NP1. Northern blot analysis suggest that the NPTX1 transcript is expressed only in the nervous system. Other studies demonstrated that rat NP1 formed heterocomplexes with Narp (Xu et al., Neuron 39:513-26, 2003), in which the proteins were covalently linked by disulfide bonds.

Neuronal pentraxin receptor (NPR) is a third member of the NP family that is primarily expressed in the central nervous system, and is physically associated with Narp and NP1 (Dodds et al., J Biol Chem 272:21499-94, 1997; Kirkpatrick et al., J Biol Chem 275:17786-92, 2000). Unlike Narp and NP1, NPR possesses an N-terminal transmembrane domain and a short (approximately 7 amino acid) intracellular sequence. The extracellular portion of NPR is highly homologous to Narp and NP1, and includes both a self-association domain and a pentraxin domain (Kirkpatrick et al., supra; Xu et al., Neuron 39:513-28, 2003).

An exemplary neuronal pentraxin receptor protein from human is provided in GenBank Accession No. NP_{—}055108 (SEQ ID NO:3). Species homologs of NPR from rat and mouse are provided in GenBank Accession Nos. NP_{—}110468 and AAK11300, respectively. In certain embodiments, NPR polypeptides may contain conservative variations in sequence while maintaining biological function. Also provided are fragments of NPR that are biologically functional. In certain embodiments, the fragment is capable of internalizing AMPA receptors, clustering AMPA receptors, or modulating metabotropic glutamate receptor (mGluR) dependent long term depression (LTD) of a synapse.

The phrases “cleavage site A” or “site A” are used interchangeably herein and refer to a site corresponding to amino acid residue L36 of SEQ ID NO:3 where the protein is cleaved. Cleavage at site A occurs N-terminal to residue L36 and results in fragments consisting of residues 1-35 and 36-494 of SEQ ID NO:3.

The phrases “cleavage site B” or “site B” are used interchangeably herein and refer to a site corresponding to amino acid residue A175 of SEQ ID NO:3 where the protein is cleaved. Cleavage at site B occurs N-terminal to residue D176 and results in fragments consisting of residues 1-175 and 176-494 of SEQ ID NO:3.

In some embodiments, the fragments are those produced by cleavage of NPR at cleavage site A or at cleavage site B of NPR. Particular fragments include the fragment containing amino acid residues 1-175 of NPR; the fragment containing amino acid residues 36-494 of NPR; and the fragment containing amino acid residues 176-494 of NPR. Exemplary fragments include residues 1-175 of SEQ ID NO:3 (SEQ ID NO:5); residues 36-494 of SEQ ID NO:3 (SEQ ID NO:7); and residues 176-494 of SEQ ID NO:3 (SEQ ID NO:9). Also provided are variants of the recited fragments. In some embodiments the variant fragments contain conservative variations. In other embodiments, the fragment is at least 90% identical, or at least 95% identical, or even at least 98% identical to a fragment having a sequence selected from the group consisting of SEQ ID NO:5, SEQ ID NO:7, and SEQ ID NO:9.

An exemplary polynucleotide encoding a human NPR is provided in GenBank Accession No. NM_{—}014293 (SEQ ID NO:4). Also provided are polynucleotides encoding NPR fragments. In certain embodiments, the polynucleotides encode the fragment containing amino acid residues 1-175 of NPR, or the fragment containing amino acid residues 36-494 of NPR, or the fragment containing amino acid residues 176-494 of NPR. Also provided are polynucleotides encoding variant fragments. In some embodiments, the polynucleotide is at least 90% identical, or at least 95% identical, or even at least 98% identical to a polynucleotide encoding a sequence selected from the group consisting of SEQ ID NO:5, SEQ ID NO:7, and SEQ ID NO:9.

Excitory amino acid receptors (EAA receptors) are the major class of excitatory neurotransmitter receptors in the central nervous system. “EAA receptors” are membrane spanning proteins that mediate the stimulatory actions of glutamate and possibly other endogenous acidic amino acids. EAA are crucial for fast excitatory neurotransmission and they have been implicated in a variety of diseases including Alzheimer's disease and epilepsy. In addition, EAA are integral to the processes of long-term potentiation, one of the synaptic mechanisms underlying learning and memory. There are three main subtypes of EAA receptors: (1) the metabotropic or trans ACPD receptors, (2) the ionotropic NMDA receptors, and (3) the non-NMDA receptors, which include the AMPA receptors.

The metabotropic glutamate receptors (mGluRs) are G-protein coupled receptors (GPCRs) and are divided into 3 groups according to agonist selectivity, coupling to different effector systems, and sequence homology. Group I includes mGluR1 and mGluR5, the activity of which receptors is mediated by a G-protein that activates a phosphatidylinositol-calcium second messenger system. Group II, which includes mGluR2 and mGluR3, and group III, which includes mGluR4, mGluR6, mGluR7, and mGluR8, act via a G-protein that inhibits adenylate cyclase activity. mGluR possess seven membrane-spanning domains and a large extracellular domain.

Ionotropic glutamate receptors are generally divided into two classes: the NMDA and non-NMDA receptors. Both classes of receptors are linked to integral cation channels and share some amino acid sequence homology. Seven non-NMDA glutamate receptor subunits have been cloned thus far. GluR1-4 are termed AMPA (α-amino-3-hydroxy-5-methylisoxazole-4-propionic acid) receptors because AMPA preferentially activates receptors composed of these subunits, while GluR5-7 are termed kainate receptors as these are preferentially sensitive to kainic acid. Thus, an “AMPA receptor” is a non-NMDA receptor that can be activated by AMPA. AMPA receptors include the GluR1-4 family, which form homo-oligomeric and hetero-oligomeric complexes which display different current-voltage relations and Ca²⁺ permeability. Polypeptides encoded by GluR1-4 nucleic acid sequences can form functional ligand-gated ion channels. Therefore, an AMPA receptor includes a receptor having a GluR1, GluR2, GluR3 or GluR4 subunit.

Also provided herein are fragments of AMPA receptors capable of binding a long pentraxin or a cadherin protein. In some embodiments, the AMPA receptor fragment is of about 10 amino acids, or about 12 amino acids, or about of about 15 amino acids, or about 25 amino acids, or about 40 amino acids, or about 50 amino acids. In some embodiments, the fragment is from a GluR1 extracellular domain. In certain embodiments, the AMPA receptor fragment is a Narp association region (NAR), for example a NAR1 peptide. An exemplary NAR1 peptide has the following sequence VDWKRPKYTSALTYDGVKVMAEAFQSLRRQRIDISRRGNAGDC (SEQ ID NO:11). In certain embodiments, the peptide is 90% identical to the sequence set forth in SEQ ID NO: 11; or the peptide is 95% identical to the sequence set forth in SEQ ID NO: 11; or the peptide is 98% identical to the sequence set forth in SEQ ID NO: 11. In one aspect, a fragment of about 12 amino acids of a NAR1 peptide is sufficient to bind long pentraxins. Such fragments include the Narp binding motif (N1M). In one aspect, the fragment has the sequence LRRQRIDISRRG (SEQ ID NO:24).

In other embodiments, the fragment is a NAR2 peptide. An exemplary NAR2 peptide has the following sequence: SVFVRTTEEGMIRVRKSKGKYAYLLESTMNEYIEQRKPCDTMKVG (SEQ ID NO:25). In certain embodiments, the peptide is 90% identical to the sequence set forth in SEQ ID NO:25; or the peptide is 95% identical to the sequence set forth in SEQ ID NO:25; or the peptide is 98% identical to the sequence set forth in SEQ ID NO:25.

Also provided herein is a nucleic acid encoding a NAR1 peptide. In some embodiments, the nucleic acid encodes a polypeptide 90% identical to the sequence set forth in SEQ ID NO: 11; or a polypeptide 95% identical to the sequence set forth in SEQ ID NO: 11; or even a polypeptide 98% identical to the sequence set forth in SEQ ID NO: 11. In one aspect, the nucleic acid encodes the amino acid sequence set forth in SEQ ID NO: 11.

Also provided herein is a nucleic acid encoding a NAR2 peptide. In some embodiments, the nucleic acid encodes a polypeptide 90% identical to the sequence set forth in SEQ ID NO:25; or a polypeptide 95% identical to the sequence set forth in SEQ ID NO:25; or even a polypeptide 98% identical to the sequence set forth in SEQ ID NO:25. In one aspect, the nucleic acid encodes the amino acid sequence set forth in SEQ ID NO:25.

Activation of an AMPA receptor results in the opening of associated Na⁺ channels and the generation of excitatory postsynaptic potentials. An “excitatory postsynaptic potential” or “EPSP” is a transient change in the membrane potential in a postsynaptic neuron caused by the binding of an excitatory neurotransmitter released by the corresponding presynaptic neuron to postsynaptic receptors. In general, the membrane potential changes in a depolarizing direction, although this is not always the case. The depolarization, during which membrane potential becomes more positive, reflects an increased excitability of the cell as the membrane potential is brought closer to the threshold for the generation of an action potential. An EPSP can be a “fast EPSP,” with an amplitude on the order of millivolts, with a latency of tens of milliseconds, and a duration of hundreds of milliseconds. However, some cells exhibit “slow EPSPs” with a long latency (approximately 0.5 seconds or more), and a prolonged time course.

Under certain conditions, AMPA receptors are known to form aggregates. An “aggregate” is a cluster of AMPA receptors. Methods for demonstrating the formation of aggregates are well known in the art (e.g., immunohistochemical methods). (See examples section for additionally methodology.) A method is provided for identifying a compound which affects the formation of AMPA receptors into aggregates. The method includes incubating the compound and a cell expressing A neuronal pentraxin under conditions sufficient to allow the compound to interact with the cell. The cell can be a pre-synaptic cell or a post-synaptic cell. The pre- or post-synaptic cell can be any cell of interest. The effect of the compound on the formation of AMPA receptors into aggregates is then determined. The effect can be determined directly in the pre-synaptic cell, or the effect can be determined in a post-synaptic cell synapsing with the pre-synaptic cell. The formation of AMPA receptors into aggregates of the cell is then compared with the formation of AMPA receptors into aggregates in a control. Suitable controls include, but are not limited to, the formation of AMPA receptors into aggregates in a pre-synaptic cell not contacted with the compound or a post-synaptic cell synapsing with the pre-synaptic cell not contacted with the compound.

The compounds which affect the aggregation of AMPA receptors can include peptides, peptidomimetics, polypeptides, pharmaceuticals, and chemical compounds and biological agents. Antibodies, neurotropic agents, and anti-epileptic compounds can also be tested using the method of the invention.

“Incubating” includes conditions which allow contact between the test agent and the cell of interest. “Contacting” includes in solution or in solid phase. Candidate compounds that affect a neuronal pentraxin include chemical compounds. One class is organic molecules, preferably small organic compounds having a molecular weight of more than 50 and less than about 2,500 Daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups.

The test agent may also be a combinatorial library for screening a plurality of compounds. Compounds such as peptides identified in the method of the invention can be further cloned, sequenced, and the like, either in solution of after binding to a solid support, by any method usually applied to the isolation of a specific DNA sequence Molecular techniques for DNA analysis (Landegren et al., Science 242:229-237, 1988) and cloning have been reviewed (Sambrook et al. Molecular Cloning: a Laboratory Manual, 2nd ed.; Cold Spring Harbor Laboratory Press, Plainview, N.Y., 1998, herein incorporated by reference).

Candidate compounds are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc., to produce structural analogs. Candidate agents are also found among biomolecules including, but not limited to: peptides, saccharides, fatty acids, steroids, purines, pyrimidines, derivatives, structural analogs or combinations thereof.

A compound can affect AMPA receptor aggregation by either stimulating or inhibiting the receptor aggregation. A compound “inhibits” AMPA receptor aggregation level of aggregation of AMPA receptors is decreased as compared with the level in the absence of the test compound. A compound “stimulates” AMPA receptor aggregation if the level of AMPA receptor aggregation is increased as compared to a control in the absence of the test compound.

A variety of other agents may be included in the screening assay. These include agents like salts, neutral proteins, e.g., albumin, detergents, etc. that are used to facilitate optimal protein-protein binding and/or reduce nonspecific or background interactions. Reagents that improve the efficiency of the assay, such as protease inhibitors, nuclease inhibitors, antimicrobial agents, etc., may be used. The mixture of components are added in any order that provides for the requisite binding. Incubations are performed at any suitable temperature, typically between 4 and 40° C. Incubation periods are selected for optimum activity, but may also be optimized to facilitate rapid high-throughput screening. Typically between 0.1 and 10 hours will be sufficient.

The sample can be any sample of interest. The sample may be a cell sample or a membrane sample prepared from a cell sample. Suitable cells include any host cells containing a neuronal pentraxin. The cells can be primary cells or cells of a cell line. In one embodiment, the sample is a primary cell, such as a neuronal cell, that expresses a neuronal pentraxin. In another embodiment, the cell is a cell line that expresses a neuronal pentraxin. Specific, non-limiting examples of cells suitable for use with the method of the invention are cultured hippocampal neurons or spinal neurons. Methods of culturing neuronal cell suitable for use in the method of the invention are known to one of skill in the art (see O'Brien et al., Neuron 21:1067-98, 1998; O'Brien et al., Curr. Opin. Neurobiol. 8:364-9, 1998; O'Brien et al., J. Neurosci. 17:7339-50, 1997; Mammen et al., J. Neurosci. 17: 7351-8, 1997; Liao et al., Nature Neurosci. 2:37-43, 1999, all herein incorporated by reference).

In yet another embodiment, the sample is a host cell transfected with a nucleic acid encoding a neuronal pentraxin. The nucleic acid encoding a neuronal pentraxin can be included in a nucleic acid encoding a fusion protein, wherein the nucleic acid encoding the neuronal pentraxin is linked to a nucleic acid encoding another polypeptide. The fusion protein can be a neuronal pentraxin polypeptide linked to a readily detectible polypeptide. A “detectible polypeptide” is any polypeptide that can be readily identified using methods well known to one of skill in the art. In one embodiment, the detectible polypeptide can be an antigen which can be specifically bound by an antibody of interest (e.g., myc antigen and an anti-myc antibody). In another embodiment, the detectible polypeptide can catalyze an enzymatic reaction (e.g., lacZ). In yet another embodiment, the detectible polypeptide can be detected by its physical parameters (e.g., fluorescence when excited with light of a specific wavelength) or spatial parameters.

DNA sequences encoding a neuronal pentraxin can be expressed in vitro by transfer of nucleic acid into a suitable host cell. “Host cells” are cells in which a vector can be propagated and its nucleic acid expressed. In certain embodiments, the host cell is eukaryotic. The term also includes any progeny of the subject host cell. It is understood that all progeny may not be identical to the parental cell since there may be mutations that occur during replication. However, such progeny are included when the term “host cell” is used. Methods of stable transfer, meaning that the foreign DNA is continuously maintained in the host, are known in the art.

In the present invention, neuronal pentraxin encoding polynucleotide sequences may be inserted into an expression vector. The term “expression vector” refers to a plasmid, virus or other vehicle known in the art that has been manipulated by insertion or incorporation of the neuronal pentraxin nucleic acid sequences. Polynucleotide sequence which encode a neuronal pentraxin can be operatively linked to expression control sequences. “Operatively linked” refers to a juxtaposition wherein the components so described are in a relationship permitting them to function in their intended manner. An expression control sequence operatively linked to a coding sequence is ligated such that expression of the coding sequence is achieved under conditions compatible with the expression control sequences. As used herein, the term “expression control sequences” refers to nucleic acid sequences that regulate the expression of a nucleic acid sequence to which it is operatively linked. Expression control sequences are operatively linked to a nucleic acid sequence when the expression control sequences control and regulate the transcription and, as appropriate, translation of the nucleic acid sequence. Thus, expression control sequences can include appropriate promoters, enhancers, transcription terminators, as start codon (i.e., ATG) in front of a protein-encoding gene, splicing signal for introns, maintenance of the correct reading frame of that gene to permit proper translation of mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter.

By “promoter” is meant minimal sequence sufficient to direct transcription. Also included in the invention are those promoter elements which are sufficient to render promoter-dependent gene expression controllable for cell-type specific, tissue-specific, or inducible by external signals or agents; such elements may be located in the 5′ or 3′ regions of the gene. Both constitutive and inducible promoters, are included in the invention (see e.g., Bitter et al., 1987, Methods in Enzymology 153:516-544). For example, when cloning in mammalian cell systems, promoters derived from the genome of mammalian cells (e.g., metallothionein promoter) or from mammalian viruses (e.g., the retrovirus long terminal repeat; the adenovirus late promoter; the vaccinia virus 7.5K promoter) may be used. Promoters produced by recombinant DNA or synthetic techniques may also be used to provide for transcription of the nucleic acid sequences of the invention.

In the present invention, the polynucleotide encoding a neuronal pentraxin may be inserted into an expression vector which contains a promoter sequence which facilitates the efficient transcription of the inserted genetic sequence of the host. The expression vector typically contains an origin of replication, a promoter, as well as specific genes which allow phenotypic selection of the transformed cells. A specific, non-limiting example of a vectors suitable for use in the present invention include, but are not limited to the pMSXND expression vector for expression in mammalian cells (Lee and Nathans, 1988, J. Biol. Chem. 263:3521), amongst others. The DNA segment can be present in the vector operably linked to regulatory elements, for example, a promoter (e.g., T7, metallothionein I, neurofilament, or polyhedrin promoters).

In yeast, a number of vectors containing constitutive or inducible promoters may be used. For a review see, Current Protocols in Molecular Biology, Vol. 2, 1988, Ed. Ausubel et al., Greene Publish. Assoc. & Wiley Interscience, Ch. 13; Grant et al., 1987, “Expression and Secretion Vectors for Yeast,” in Methods in Enzymology, Eds. Wu & Grossman, 31987, Acad. Press, N.Y., Vol. 153, pp. 516-544; Glover, 1986, DNA Cloning, Vol. II, IRL Press, Wash., D.C., Ch. 3; and Bitter, 1987, “Heterologous Gene Expression in Yeast,” Methods in Enzymology, Eds. Berger & Kimmel, Acad. Press, N.Y., Vol. 152, pp. 673-684; and The Molecular Biology of the Yeast Saccharomyces, 1982, Eds. Strathem et al., Cold Spring Harbor Press, Vols. I and II. A constitutive yeast promoter such as ADH or LEU2 or an inducible promoter such as GAL may be used (“Cloning in Yeast,” Ch. 3, R. Rothstein In: DNA Cloning Vol. 11, A Practical Approach, Ed. D. M. Glover, 1986, IRL Press, Wash., D.C.). Alternatively, vectors may be used which promote integration of foreign nucleic acid sequences into the yeast chromosome. Yeast may be used to search for molecules that disrupt interaction between neuronal pentraxin molecules or neuronal pentraxin and AMPA receptor, for example, by co-expressing a neuronal pentraxin and AMPA receptor or fragments thereof, that interact.

Mammalian expression systems which utilize recombinant viruses or viral elements to direct expression may be engineered. For example, when using adenovirus expression vectors, the neuronal pentraxin coding sequence may be ligated to an adenovirus transcription/translation control complex, e.g., the late promoter and tripartite leader sequence. Alternatively, the vaccinia virus 7.5K promoter may be used (e.g., see, Maced. et al., 1982, Proc. Natl. Acad. Sc. USA 79:7415-7419; Maced. et al., 1984, J. Biol. 49:857-864; Panniculi et al., 1982, Proc. Natl. Acad. Sc. USA 79:4927-4931). Of particular interest are vectors based on bovine papilloma virus which have the ability to replicate as extrachromosomal elements (Salver, et al., 1981, Mol. Cell. Biol. 1:486). Shortly after entry of this nucleic acid into mouse cells, the plasmid replicates to about 100 to 200 copies per cell. Transcription of the inserted cDNA does not require integration of the plasmid into the host's chromosome, thereby yielding a high level of expression. These vectors can be used for stable expression by including a selectable marker in the plasmid, such as, for example, the neo gene. Alternatively, the retroviral genome can be modified for use as a vector capable of introducing and directing the expression of the a neuronal pentraxin gene in host cells (Cone & Mulligan, 1984, Proc. Natl. Acad. Sc. USA 81:6349-6353). High level expression may also be achieved using inducible promoters, including, but not limited to, the metallothionein I.A. promoter and heat shock promoters.

Hosts can include yeast and mammalian organisms. Methods of expressing DNA sequences having eukaryotic or viral sequences in eukaryotes are well known in the art. In addition, prokaryotic cells, such as bacterial cells can be used for the production of a neuronal pentraxin protein of use with the subject invention. Biologically functional viral and plasmid DNA vectors capable of expression and replication in a host are known in the art. Such vectors are used to incorporate nucleic acid sequences of the use with the invention.

For long-term, high-yield production of recombinant proteins, stable expression is desirable. Rather than using expression vectors which contain viral origins of replication, host cells can be transformed with a neuronal pentraxin cDNA controlled by appropriate expression control elements (e.g., promoter, enhancer, sequences, transcription terminators, polyadenylation sites, etc.), and a selectable marker. The selectable marker in the recombinant plasmid confers resistance to the selection and allows cells to stably integrate the plasmid into their chromosomes and grow to form foci which in turn can be cloned and expanded into cell lines. For example, following the introduction of foreign nucleic acid, engineered cells may be allowed to grow for 1-2 days in an enriched media, and then are switched to a selective media. A number of selection systems may be used, including, but not limited to the herpes simplex virus thymidine kinase gene (Wigler, et al., 1977, Cell 11:223), hypoxanthine-guanine phosphoribosyltransferase gene (Szybalska & Szybalski, 1962, Proc. Natl. Acad. Sci. USA 48:2026), and the adenine phosphoribosyltransferase [Lowy, et al., 1980, Cell 22:817) genes can be employed in tk-, hgprt or aprt cells respectively. Additionally, antimetabolite resistance can be used as the basis of selection for which confers resistance to methotrexate (Wigler, et al., 1980, Natl. Acad. Sci. USA 77:3567; O'Hare, et al., 1981, Proc. Natl. Acad. Sci. USA 78: 1527); the gpt gene, which coders resistance to mycophenolic acid (Mulligan & Berg, 1981, Proc. Natl. Acad. Sci. USA 78:2072; the neo gene, which confers resistance to the aminoglycoside G-418 (Colberre-Garapin, et al., 1981, J. Mol. Biol. 150:1); and the hygro gene, which confers resistance to hygromycin (Santerre, et al., 1984, Gene 30:147) genes. Recently, additional selectable genes have been described, namely trpB, which allows cells to utilize indole in place of tryptophan; hisD, which allows cells to utilize histinol in place of histidine (Hartman & Mulligan, 1988, Proc. Natl. Acad. Sci. USA 85:8047); and ODC (ornithine decarboxylase) which confers resistance to the ornithine decarboxylase inhibitor, 2-(difluoromethyl)-DL-ornithine, DFMO (McConlogue L., 1987, In: Current Communications in Molecular Biology, Cold Spring Harbor Laboratory ed.).

By “transformation” is meant a genetic change induce in a cell following incorporation of new DNA (i.e., DNA exogenous to the cell). Where the cell is a mammalian cell, the genetic change is generally achieved by introduction of the DNA into the genome of the cell (i.e., stable).

By “transformed cell” is meant a cell into which (or into an ancestor of which has been introduced, by means of recombinant DNA techniques, a nucleic acid molecule encoding a neuronal pentraxin. Transformation of a host cell with recombinant DNA may be carried out by conventional techniques as are well known to those skilled in the art. Where the host is prokaryotic, such as E. coli, and is used for the production of a neuronal pentraxin polypeptide, competent cells which are capable of DNA uptake can be prepared from cells harvested after exponential growth phase and subsequently treated by the CaCl₂ method using procedures well known in the art. Alternatively, MgCl₂ or RbCl can be used. Transformation can also be performed after forming a protoplast of the host cell if desired. Vectors for the transformation of prokaryotic cells are well known to one of skill in the art (see Sambrook et al., supra).

When the host is a eukaryote, such methods of transfection of DNA as calcium phosphate co-precipitates, conventional mechanical procedures such as microinjection, electroporation insertion of a plasmid encased in liposomes, or virus vectors may be used. Eukaryotic cells can also be cotransformed with DNA sequences encoding a neuronal pentraxin, and a second foreign DNA molecule encoding a selectable phenotype, such as the herpes simplex thymidine kinase gene. Another method is to use a eukaryotic viral vector, such as simian virus 40 (SV40) or bovine papilloma virus, to transiently infect or transform eukaryotic cells and express the protein (see for example, Eukaryotic Viral Vectors, Cold Spring Harbor Laboratory, Gluzman ed., 1982).

In another embodiment, a method is provided for identifying a compound which affects the formation of synaptic connections. The method includes incubating the compound and a cell expressing a neuronal pentraxin under conditions sufficient to allow the compound to interact with the cell, determining the effect of the compound on the formation of synaptic connections, and comparing the synaptic connections of the cell contacted with the compound with the synaptic connections of a cell not contacted with said compound. A “synaptic connection” or a “synapse” is a specialized junction between two nerve cells or between a nerve and a muscle cell across which signals are transmitted. A synapse can be a chemical synapse or an electrical synapse. A “chemical synapse” is a synapse where the plasma membrane of the axon terminal of the transmitting neuron and that of the receiving cell are separated by a small gap. Chemical neurotransmitters are released from the axon terminal, diffuse across the synapse, and stimulate receptors on the postsynaptic membrane. An “electrical synapse” is a synapse where an electrical signal carried by ions is transmitted from one cell to another by a gap junction. Methods for analyzing and quantitating synaptic connections are well known to one of skill in the art and provided in the Examples as well.

A compound can affect synapse formation by either stimulating or inhibiting the formation of synaptic connections. A compound “inhibits” formation of synaptic connections if the level of formation of synaptic connections is decreased as compared with the level in the absence of the test compound. A compound “stimulates” formation of synaptic connections if the level of formation of synaptic connections is increased as compared to a control in the absence of the test compound.

The sample can be any sample of interest. The sample may be a cell sample or a membrane sample prepared from a cell sample. Suitable cells include any host cells containing a neuronal pentraxin. The cells can be primary cells or cells of a cell line that express a neuronal pentraxin. In one embodiment, the sample is a primary cell, such as a neuronal cell, that expresses a neuronal pentraxin. In another embodiment, the cell is a cell line that expresses a neuronal pentraxin. In yet another embodiment, the sample is a host cell transfected with a nucleic acid encoding a neuronal pentraxin. The nucleic acid encoding the neuronal pentraxin may be including in a nucleic acid encoding a fusion protein, wherein the nucleic acid encoding the neuronal pentraxin is linked to a nucleic acid encoding another polypeptide.

A method is also provided for identifying a compound that modulates the expression of an immediate early gene. The method includes contacting a test compound with a sample containing a nucleic acid encoding a neuronal pentraxin; and determining whether the test compound affects the expression of the immediate early gene nucleic acid, wherein the presence of an effect indicates that the test compound modulates immediate early gene expression.

Immediate early genes (IEGs) are rapidly induced in neurons by neurotransmitter stimulation and synaptic activity and are hypothesized to be part of the macromolecular response required for long-term plasticity (Goelet, et al., supra; Sheng and Greenberg, Neuron, 4:477, 1990; Silva and Giese, Neurobiology, 4:413, 1994). To identify cellular mechanisms that may contribute to long-term plasticity in the vertebrate brain, differential cloning techniques have been used to identify genes that are rapidly induced by depolarizing stimuli (Nedivi, et al., Nature, 363:713, 1993; Qian, et al., Nature, 361:453, 1993; Yamagata, et al., Neuron, 11:371, 1993; Yamagata, et al., Learning and Memory 1:140, 1994; Yamagata, et al., Journal of Biological Chemistry, 269:16333, 1994; Andreasson and Worley, Neuroscience, 69:781, 1995; Lyford, et al., Neuron, 14:433, 1995). In contrast to the earlier focus on transcription factors, many of the newly characterized IEGs represent molecules that can directly modify the function of cells and include growth factors (Nedivi, et al., supra; Andreasson and Worley, supra), secreted enzymes that can modify the extracellular matrix, such as tissue plasminogen activator (Qian, et al., supra), enzymes involved in intracellular signaling, such as prostaglandin synthase (Yamagata, et al., supra), and a novel homolog of H-Ras, termed Rheb (Yamagata, et al., supra), as well as a novel cytoskeleton-associated protein, termed Arc (Lyford, et al., supra). The remarkable functional diversity of this set of rapid response genes is representative of the repertoire of cellular mechanisms that are likely to contribute to activity-dependent neuronal plasticity.

An “immediate early gene” or an “IEG” is a gene whose expression is rapidly increased immediately following a stimulus. For example, genes expressed by neurons that exhibit a rapid increase in expression immediately following neuronal stimulation are neuronal IEGs. Such neuronal IEGs have been found to encode a wide variety of polypeptides including transcription factors, cytoskeletal polypeptides, growth factors, and metabolic enzymes as well as polypeptides involved in signal transduction. The identification of neuronal IEGs and the polypeptides they encode provides important information about the function of neurons in, for example, learning, memory, synaptic transmission, tolerance, and neuronal plasticity.

In another embodiment, a method is provided for increasing the number of excitatory synapses of a neuron, including introducing into the neuron a polynucleotide sequence encoding a neuronal pentraxin, operatively linked to a promoter, thereby increasing the number of excitory synapses of the neuron. The polynucleotide sequence encoding a neuronal pentraxin may be introduced into the neuron in vitro. Alternatively, the polynucleotide sequence encoding a neuronal pentraxin can be introduced into the neuron in vivo. A method is also provided for increasing the number of excitory synapses of a neuron, including introducing into the neuron a neuronal pentraxin polypeptide, thereby increasing the number of excitory synapses of the neuron.

A method is further provided for treating a subject with a disorder associated with a decrease in a function or expression of a neuronal pentraxin, including administering to the subject a therapeutically effective amount of a compound that augments neuronal pentraxin function or expression. In yet another embodiment, a method is provided for treating a subject with a disorder associated with a decrease in function or expression of a neuronal pentraxin, including administering to the subject a therapeutically effective amount of a polynucleotide encoding a neuronal pentraxin.

Essentially, any disorder which is etiologically linked to increased expression of a neuronal pentraxin could be considered susceptible to treatment with an agent that inhibits the neuronal pentraxin expression or activity. Any disorder which is etiologically linked to decreased expression of a neuronal pentraxin could be considered susceptible to treatment with an agent that stimulates a neuronal pentraxin expression or activity. The disorder may be a neuronal cell disorder. Examples of neuronal cell disorders include but are not limited to Alzheimer's disease, Parkinson's disease, stroke, epilepsy, neurodegenerative disease, Huntington's disease, and brain or spinal cord injury/damage, including ischemic injury.

Detection of altered (decreased or increased) levels of a neuronal pentraxin expression can be accomplished by hybridization of nucleic acids isolated from a cell of interest with a neuronal pentraxin polynucleotide of the invention. Analysis, such as Northern Blot analysis, are utilized to quantitate expression of the neuronal pentraxin, such as to measure neuronal pentraxin transcripts. Other standard nucleic acid detection techniques will be known to those of skill in the art. Detection of altered levels of neuronal pentraxin can also accomplished using assays designed to detect the neuronal pentraxin polypeptide. For example, antibodies that specifically bind NP1, Narp, or NPR polypeptide can be utilized. Analyses, such as radioimmune assay or immunohistochemistry, are then used to measure the neuronal pentraxin, such as to measure protein concentration qualitatively or quantitatively.

Treatment can include modulation of neuronal pentraxin gene expression or neuronal pentraxin activity by administration of a therapeutically effective amount of a compound that modulates the neuronal pentraxin. The term “modulate” envisions the suppression of neuronal pentraxin activity or expression when neuronal pentraxin is overexpressed or has an increased activity as compared to a control. The term “modulate” also includes the augmentation of the expression of neuronal pentraxin when it is underexpressed or has a decreased activity as compared to a control. The term “compound” as used herein describes any molecule, e.g., protein, nucleic acid, or pharmaceutical, with the capability of altering the expression of neuronal pentraxin polynucleotide or activity of neuronal pentraxin polypeptide.

Candidate agents include nucleic acids encoding a neuronal pentraxin, or that interfere with expression of a neuronal pentraxin, such as an antisense nucleic acid. Candidate agents also encompass numerous chemical classes wherein the agent modulates neuronal pentraxin expression or activity.

Where a disorder is associated with the increased expression of a neuronal pentraxin, nucleic acid sequences that interfere with the expression of the neuronal pentraxin can be used. In this manner, the clustering of AMPA receptors, or the formation of excitory synapses, can be inhibited. This approach also utilizes, for example, antisense nucleic acid, ribozymes, or triplex agents to block transcription or translation of neuronal pentraxin mRNA, either by masking that mRNA with an antisense nucleic acid or triplex agent, or by cleaving it with a ribozyme in disorders associated with increased neuronal pentraxin. Alternatively, a dominant negative form of a neuronal pentraxin polypeptide could be administered. In one embodiment an agent which decreases neuronal pentraxin expression can be administered to a subject having a disorder associated with increased number of synapses. Such disorders are associated with central or peripheral nervous tissue. In one specific, non-limiting example the disorder is epilepsy. In one specific non-limiting example the disorder is a stroke.

When a neuronal pentraxin is overexpressed, candidate agents include antisense nucleic acid sequences. Antisense nucleic acids are DNA or RNA molecules that are complementary to at least a portion of a specific mRNA molecule (Weintraub, 1990, Scientific American, 262:40). In the cell, the antisense nucleic acids hybridize to the corresponding mRNA, forming a double-stranded molecule. The antisense nucleic acids interfere with the translation of the mRNA, since the cell will not translate a mRNA that is double-stranded. Antisense oligomers of about 15 nucleotides are preferred, since they are easily synthesized and are less likely to cause problems than larger molecules when introduced into the target cell. The use of antisense methods to inhibit the in vitro translation of genes is well known in the art (Marcus-Sakura, 1988, Anat. Biochem., 172:289).

Use of an oligonucleotide to stall transcription is known as the triplex strategy since the oligomer winds around double-helical DNA, forming a three-strand helix. Therefore, these triplex compounds can be designed to recognize a unique site on a chosen gene (Maher, et al., 1991, Antisense Res. and Dev., 1(3):227; Helene, C., 1991, Anticancer Drug Design, 6(6):569).

Ribozymes are RNA molecules possessing the ability to specifically cleave other single-stranded RNA in a manner analogous to DNA restriction endonucleases. Through the modification of nucleotide sequences which encode these RNAs, it is possible to engineer molecules that recognize specific nucleotide sequences in an RNA molecule and cleave it (Cech, 1988, J. Amer. Med. Assn., 260:3030). A major advantage of this approach is that, because they are sequence-specific, only mRNAs with particular sequences are inactivated.

There are two basic types of ribozymes namely, tetrahymena-type (Hasselhoff, 1988, Nature, 334:585) and “hammerhead”-type. Tetrahymena-type ribozymes recognize sequences which are four bases in length, while “hammerhead”-type ribozymes recognize base sequences 11-18 bases in length. The longer the recognition sequence, the greater the likelihood that the sequence will occur exclusively in the target mRNA species. Consequently, hammerhead-type ribozymes are preferable to tetrahymena-type ribozymes for inactivating a specific mRNA species and 18-based recognition sequences are preferable to shorter recognition sequences.

Where a disorder is associated with the decreased expression of a neuronal pentraxin, nucleic acid sequences that encode the neuronal pentraxin can be used. For example, an agent which modulates Narp expression includes a polynucleotide encoding the polypeptide of SEQ ID NO:2, or a conservative variant thereof. Similarly, an agent which modulates NP1 or NPR expression includes a polynucleotide encoding the polypeptide of SEQ ID NO: 13 (GenBank Accession No. NP_{—}002513) or SEQ ID NO:3, respectively, or a conservative variants or a functional fragment thereof. Alternatively, an agent for use with the subject invention includes agents that increase the expression of a polynucleotide encoding a neuronal pentraxin or an agent that increases the activity of neuronal pentraxin polypeptide. In one embodiment, an agent which increases Narp expression or function is administered to a subject having a disorder associated with a decreased number of synapses.

The method of treating a subject with a neuronal disorder associated with a decreased expression of a neuronal pentraxin includes intracerebral grafting of neurons expressing the neuronal pentraxin to the region of the CNS having the disorder. Where necessary, the neuron can be genetically engineered to contain a second exogenous gene. The disorder may be from either disease or trauma (injury). Neuronal transplantation, or “grafting” involves transplantation of cells into the central nervous system or into the ventricular cavities or subdurally onto the surface of a host brain. Such methods for grafting will be known to those skilled in the art and are described in Neural Grafting in the Mammalian CNS, Bjorklund and Stenevi, eds., (1985), and U.S. Pat. No. 5,082,670 incorporated by reference herein. Procedures include intraparenchymal transplantation, (i.e., within the host brain) achieved by injection or deposition of tissue within the host brain so as to be apposed to the brain parenchyma at the time of transplantation.

Administration of the neurons into selected regions of the recipient subject's brain may be made by drilling a hole and piercing the dura to permit the needle of a microsyringe to be inserted. The neurons can alternatively be injected intrathecally into the spinal cord region. The neuronal preparation permits grafting of neurons to any predetermined site in the brain or spinal cord, and allows multiple grafting simultaneously in several different sites using the same cell suspension and permits mixtures of cells from different anatomical regions. The present invention provides a method for transplanting various neural tissues, by providing previously unavailable neurons in order to grow a sufficient number of cells for in vitro gene transfer followed by in vivo implantation.

The neuron used for treatment of a neuronal disorder associated with decreased function or expression of a neuronal pentraxin may optionally contain a second exogenous gene, for example, an oncogene, a gene which encodes a receptor, or a gene which (encodes a ligand, and/or a neuronal pentraxin-encoding polynucleotide. Such receptors include receptors which respond to dopamine, GABA, adrenaline, noradrenaline, serotonin, glutamate, acetylcholine and other neuropeptides, as described above. Examples of ligands which may provide a therapeutic effect in a neuronal disorder include dopamine, adrenaline, noradrenaline, acetylcholine, gamma-aminobutyric acid and serotonin. The diffusion and uptake of a required ligand after secretion by a donor neuroblast would be beneficial in a disorder where the subject's neural cell is defective in the production of such a gene product. A neuron genetically modified to secrete a neurotrophic factor, such as nerve growth factor, (NGF), or a neuronal pentraxin might be used to prevent degeneration of cholinergic neurons that might otherwise die without treatment. Alternatively, neurons to be grafted into a subject with a disorder of the basal ganglia, such as Parkinson's disease, can be modified to contain an exogenous gene encoding a neuronal pentraxin, and/or L-DOPA, the precursor to dopamine. Parkinson's disease is characterized by a loss of dopamine neurons in the substantia-nigra of the midbrain, which have the basal ganglia as their major target organ. Alternatively, neurons derived from substantia-nigra neuronal cells which produce dopamine could be introduced into a Parkinson's patient brain to provide cells which “naturally” produce dopamine.

Other neuronal disorders that may be associated with a decreased expression of a neuronal pentraxin, that can be treated by the method of the invention, include Alzheimer's disease, Huntington's disease, neuronal damage due to stroke, and damage in the spinal cord. Alzheimer's disease is characterized by degeneration of the cholinergic neurons of the basal forebrain. The neurotransmitter for these neurons is acetylcholine, which is necessary for their survival. Engraftment of cholinergic neurons, or neurons containing an exogenous gene for a neuronal pentraxin which would promote synaptogenesis of neurons can be accomplished by the method of the invention, as described. Following a stroke, there is selective loss of cells in the CA1 of the hippocampus as well as cortical cell loss which may underlie cognitive function and memory loss in these patients. Engraftment of neurons expressing a neuronal pentraxin, or therapy with nucleic acid sequences encoding a neuronal pentraxin, can be used to increase the number of synapses or the clustering of AMPA receptors, in selected regions of the nervous system. The engraftment of neurons may affect learning or memory.

The method of treating a subject with a neuronal disorder also contemplates the grafting of neurons expressing a neuronal pentraxin in combination with other therapeutic procedures useful in the treatment of disorders of the CNS. For example, the neurons can be co-administered with agents such as growth factors, gangliosides, antibiotics, neurotransmitters, neurohormones, neurotrophins, toxins, neurite promoting molecules and antimetabolites and precursors of these molecules such as the precursor of dopamine, L-DOPA.

The present invention also provides gene therapy for the treatment of disorders which are associated with a neuronal pentraxin. Such therapy would achieve its therapeutic effect by introduction of a therapeutic polynucleotide into cells in vivo having the disorder or introducing the therapeutic polynucleotide into cells ex vivo and then reintroducing the cells into the subject. The “therapeutic polynucleotide” may be polynucleotide sequences encoding the neuronal pentraxin, or antisense polynucleotide specific for the neuronal pentraxin, designed to treat a neuronal pentraxin-associated disorder. Polynucleotides encoding dominant negative forms of neuronal pentraxin polypeptides or antisense polynucleotides specific for neuronal pentraxins are also included.

Delivery of the therapeutic polynucleotide can be achieved using a recombinant expression vector such as a chimeric virus or a colloidal dispersion system. In particular aspects of therapeutic delivery of antisense sequences, or neuronal pentraxin polynucleotides, viral vectors or targeted liposomes are used.

Various viral vectors which can be utilized for gene therapy as taught herein include adenovirus, herpes virus, vaccinia, or, preferably, an RNA virus such as a retrovirus. Preferably, the retroviral vector is a derivative of a murine or avian retrovirus. Examples of retroviral vectors in which a single foreign gene can be inserted include, but are not limited to: Moloney murine leukemia virus (MoMuLV), Harvey murine sarcoma virus (HaMuSV), murine mammary tumor virus (MuMTV), Rous Sarcoma Virus (RSV) or lentiviral vectors (e.g., derived from HIV or FIV, for example). Preferably, when the subject is a human, a vector such as the gibbon ape leukemia virus (GaLV) is utilized. A number of additional retroviral vectors can incorporate multiple genes. All of these vectors can transfer or incorporate a gene for a selectable marker so that transduced cells can be identified and generated. By inserting a DNA sequence encoding a neuronal pentraxin of interest into the viral vector, along with another gene which encodes the ligand for a receptor on a specific target cell, for example, the vector is now target specific. Retroviral vectors can be made target specific by attaching, for example, a sugar, a glycolipid, or a protein. Preferred targeting is accomplished by using an antibody to target the retroviral vector. Those of skill in the art will know of, or can readily ascertain without undue experimentation, specific polynucleotide sequences which can be inserted into the retroviral genome or attached to a viral envelope to allow target specific delivery of the retroviral vector containing the neuronal pentraxin polynucleotide.

Since recombinant retroviruses are defective, they require assistance in order to produce infectious vector particles. This assistance can be provided, for example, by using helper cell lines that contain plasmids encoding all of the structural genes of the retrovirus under the control of regulatory sequences within the LTR. These plasmids are missing a nucleotide sequence which enables the packaging mechanism to recognize an RNA transcript for encapsidation. Helper cell lines which have deletions of the packaging signal include, but are not limited to Q2, PA317, and PA12, for example. These cell lines produce empty virions, since no genome is packaged. If a retroviral vector is introduced into such cells in which the packaging signal is intact, but the structural genes are replaced by other genes of interest, the vector can be packaged and vector virion produced.

Alternatively, NIH 3T3 or other tissue culture cells can be directly transfected with plasmids encoding the retroviral structural genes gag, pol and env, by conventional calcium phosphate transfection. These cells are then transfected with the vector plasmid containing the genes of interest. The resulting cells release the retroviral vector into the culture medium.

Another targeted delivery system for the therapeutic polynucleotides is a colloidal dispersion system. Colloidal dispersion systems include macromolecule complexes, nanocapsules, microspheres, beads, and lipid-based systems including oil-in-water emulsions, micelles, mixed micelles, and liposomes. The preferred colloidal system of this invention is a liposome. Liposomes are artificial membrane vesicles which are useful as delivery vehicles in vitro and in vivo. It has been shown that large unilamellar vesicles (LUV), which range in size from 0.2-4.0 ™ can encapsulate a substantial percentage of an aqueous buffer containing large macromolecules. RNA, DNA and intact virions can be encapsulated within the aqueous interior and be delivered to cells in a biologically active form (Fraley et al., 1981, Trends Biochem. Sci., 6:77). In addition to mammalian cells, liposomes have been used for delivery of polynucleotides in plant, yeast and bacterial cells. In order for a liposome to be an efficient gene transfer vehicle, the following characteristics should be present: (1) encapsulation of the genes of interest at high efficiency while not compromising their biological activity; (2) preferential and substantial binding to a target cell in comparison to non-target cells; (3) delivery of the aqueous contents of the vesicle to the target cell cytoplasm at high efficiency; and (4) accurate and effective expression of genetic information (Mannino, et al., 1988, Biotechniques, 6:682).

The composition of the liposome is usually a combination of phospholipids, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations.

Examples of lipids useful in liposome production include phosphatidyl compounds, such as phosphatidylglycerol, phosphatidylcholine, phosphatidiylserine, phosphatidylethanolamine, sphingo-lipids, cerebrosides, and gangliosides. Particularly useful are diacylphosphatidyl-glycerols, where the lipid moiety contains from 14-18 carbon atoms, particularly from 16-18 carbon atoms, and is saturated. Illustrative phospholipids include egg phosphatidylcholine, dipalmitoylphosphatidylcholine and distearoylphosphatidylcholine.

The targeting of liposomes can be classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticulo-endothelial system (RES) in organs which contain sinusoidal capillaries. Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization.

The surface of the targeted delivery system may be modified in a variety of ways. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand.

This invention involves administering to a subject a therapeutically effective dose of a pharmaceutical composition containing the compounds of the present invention and a pharmaceutically acceptable carrier. “Administering” the pharmaceutical composition of the present invention may be accomplished by any means known to the skilled artisan.

The pharmaceutical compositions are preferably prepared and administered in dose units. Solid dose units are tablets, capsules and suppositories. For treatment of a patient, depending on activity of the compound, manner of administration, nature and severity of the disorder, age and body weight of the patient, different daily doses are necessary. Under certain circumstances, however, higher or lower daily doses may be appropriate. The administration of the daily dose can be carried out both by single administration in the form of an individual dose unit or else several smaller dose units and also by multiple administration of subdivided doses at specific intervals.

The pharmaceutical compositions according to the invention are in general administered topically, intravenously, orally or parenterally or as implants, but even rectal use is possible in principle. Suitable solid or liquid pharmaceutical preparation forms are, for example, granules, powders, tablets, coated tablets, (micro) capsules, suppositories, syrups, emulsions, suspensions, creams, aerosols, drops or injectable solution in ampule form and also preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of present methods for drug delivery, see Langer, 1990, Science, 249:1527-1533, which is incorporated herein by reference.

The pharmaceutical compositions according to the invention may be administered locally or systemically. By “therapeutically effective dose” is meant the quantity of a compound according to the invention necessary to prevent, to cure or at least partially arrest the symptoms of the disorder and its complications. Amounts effective for this use will, of course, depend on the severity of the disease and the weight and general state of the patient. Typically, dosages used in vitro may provide useful guidance in the amounts useful for in situ administration of the pharmaceutical composition, and animal models may be used to determine effective dosages for treatment of particular disorders. Various considerations are described, e.g., in Gilman et al., eds., 1990, Goodman And Gilman's: The Pharmacological Bases of Therapeutics, 8th ed., Pergamon Press; and Remington's Pharmaceutical Sciences, 1990, 17th ed., Mack Publishing Co., Easton, Pa., each of which is herein incorporated by reference.

Provided herein are antibodies or fragments thereof that bind AMPA receptors and thereby block binding of a neuronal pentraxin to the AMPAR. In certain embodiments, the antibody or fragment thereof binds the NAR1 region of AMPAR and blocks binding of a neuronal pentraxin. In one aspect, the antibody is generated using the peptide or a portion thereof of the NAR1 region of AMPAR as set forth in SEQ ID NO: 11.

The term “antibody” as used herein includes polyclonal and monoclonal antibodies, chimeric, single chain, and humanized antibodies, as well as intact molecules and fragments thereof, such as Fab, F(ab′)2, and Fv which are capable of binding to an epitopic determinant present in an AMPA receptor. Such antibody fragments retain some ability to selectively bind with its antigen.

Methods of making antibodies and antibody fragments are known in the art. (See for example, Harlow and Lane, Antibodies: A Laboratory Manual, Cold Spring Harbor Laboratory, New York (1988), incorporated herein by reference). Monoclonal antibodies are made from antigen containing fragments of the protein by methods well known to those skilled in the art (Kohler & Milstein, Nature 256:495 (1975); Coligan et al., sections 2.5.1-2.6.7; and Harlow et al., Antibodies: A Laboratory Manual, page 726 (Cold Spring Harbor Pub. 1988), which are hereby incorporated by reference. Briefly, monoclonal antibodies can be obtained by injecting mice with a composition including an antigen/ligand, verifying the presence of antibody production by analyzing a serum sample, removing the spleen to obtain B lymphocytes, fusing the B lymphocytes with myeloma cells to produce hybridomas, cloning the hybridomas, selecting positive clones that produce antibodies to the antigen, and isolating the antibodies from the hybridoma cultures. Monoclonal antibodies can be isolated and purified from hybridoma cultures by a variety of well-established techniques. Such isolation techniques include affinity chromatography with Protein-A Sepharose, size-exclusion chromatography, and ion-exchange chromatography. See, e.g., Coligan et al., sections 2.7.1-2.7.12 and sections 2.9.1-2.9.3; Barnes et al., “Purification of Immunoglobulin G (IgG)” in Methods In Molecular Biology, Vol. 10, pages 79-104 (Humana Press 1992).

Antibodies that bind to AMPA receptors and block neuronal pentraxin binding can be prepared using a portion of the AMPAR protein where such neuronal pentraxins bind (e.g., the NAR1 region) or a portion thereof as the immunizing antigen. For the preparation of polyclonal antibodies, the polypeptide or peptide used to immunize an animal is derived from translated cDNA or chemically synthesized and can be conjugated to a carrier protein, if desired. Commonly used carrier proteins which may be chemically coupled to the immunizing peptide include keyhole limpet hemocyanin (KLH), thyroglobulin, bovine serum albumin (BSA), tetanus toxoid, and the like.

Invention polyclonal or monoclonal antibodies can be further purified, for example, by binding to and elution from a matrix to which the polypeptide or a peptide to which the antibodies were raised is bound. Those of skill in the art will know of various techniques common in the immunology arts for purification and/or concentration of polyclonal antibodies, as well as monoclonal antibodies (See, for example, Coligan, et al., Unit 9, Current Protocols in Immunology, Wiley Interscience, 1994, incorporated herein by reference).

The following examples are intended to illustrate but not to limit the invention in any manner, shape, or form, either explicitly or implicitly. While they are typical of those that might be used, other procedures, methodologies, or techniques known to those skilled in the art may alternatively be used.

### EXAMPLE 1

### NARP is Enriched at Excitatory Synapses in a Subpopulation of Neurons from Hippocampus and Spinal Cord

To study Narp protein, a rabbit polyclonal antibody was generated against a full length GST fusion protein of Narp. On western blot, this antibody recognized a single broad protein band centered at 56 kDa in rat brain, similar to the size of recombinant Narp expressed in detergent extracts of transfected HEK-293 cells. Narp protein was also detected as a similar sized triplet in rat testes, but not in other peripheral tissues. This restricted distribution of Narp protein parallels the Narp mRNA expression previously reported (Tsui et al., supra, 1996). The broadness of the Narp band in brain is consistent with the observation that Narp is glycosylated (Tsui et al., supra, 1996). In non-reducing gels, Narp migrates as a multimer, with a size greater than 220 kDa, consistent with the known ability of members of the pentraxin family to covalently multimerize through disulfide bonds (Emsley et al., Nature 367, 338-45, 1994; Gewurz et al., Curr. Opin. Immunol. 7:54-64, 1995; Bottazzi et al., J. Biol. Chem. 272:32817-23, 1997). Like Narp mRNA expression, Narp protein levels are significantly increased in adult rat hippocampus following seizure induction.

Maximal electroconvulsive seizure (MECS) resulted in a robust increase in Narp protein (8 to 12 fold at 12 hrs; n=2) without a change in the level of the constitutively expressed protein actin. The time course of Narp protein expression is typical for an immediate early gene (IEG) in that it is increased within 30 min of MECS, but is unique among known neuronal IEGs in that it remains elevated for more than 24 hrs. The prolonged increase is not associated with a prolonged increase in mRNA since Narp mRNA returns to basal levels by 8 hrs after MECS (Tsui et al., supra, 1996), suggesting that the Narp protein may be relatively stable. Immunohistochemistry in rat hippocampus showed that Narp protein is expressed in the cell bodies of most neurons throughout the hippocampus, including the dentate gyrus and CA1 region, paralleling the distribution of Narp mRNA (Tsui et al., supra, 1996). Four hours after induction of either MECS or long-term potentiation in vivo, Narp immunostaining showed a large increase throughout the dentate gyms, with little change in CA1. The hippocampal neuropil was lacking in intense Narp staining except in the hilus of the dentate gyrus, the site of termination of many of the mossy fibers. Of note, hippocampal interneurons, defined by their location and lack of dendritic spines (Buckmaster and Soltesz, Hippocampus 6:330-9, 1996), showed a clear dendritic staining pattern that was nearly as intense as that seen in the granule cell layer.

In cultured cells, the anti-Narp antibody recognized a protein with a molecular mass of 54 kD in hippocampal neurons and 58 kD in spinal neurons. Recognition of these proteins by the Narp antibody was blocked by preincubation of the antibody with Narp fusion protein. A second, minor protein at 140 kD was also seen in immunoblots of neuronal cultures, however, this protein was not blocked by preabsortion of the antibody with antigen. The 140 kD protein was not observed in immunoblots of cell surface proteins isolated by surface biotinylation techniques, indicating that it is an intracellular protein, non-specifically recognized by the rabbit serum. Of note, immunoblots of the media overlying neuronal cultures and Narp transfected 293 cells indicated that a substantial amount of Narp was secreted into the media, consistent with the observations of Tsui et al. (supra, 1996), and in keeping with the characteristics of the family of pentraxins.

The immunohistochemical distribution of Narp in cultures of postnatal rat hippocampal neurons, was quite striking, and paralleled and extended the results observed in vivo. Aspiny neurons, which are composed predominantly of inhibitory interneurons (Craig et al., Proc. Natl. Acad. Sci. USA 91:12373-7, 1994; Buckmaster and Soltesz, supra, 1996), and make up about one third of the neurons present in these cultures, displayed a somato-dendritic staining pattern for Narp with large superimposed clusters which showed a tight colocalization with GluR1 but not GAD. Greater than 90% of GluR1 clusters on aspiny neurons had associated Narp staining while only 4 of 111 GAD positive synapses contained Narp. Since our previous, and ongoing, work has shown that dendritic GluR1 clusters are almost always synaptic (defined by synaptophysin immunostaining; O'Brien et al., supra, 1997; Mammen et al., J. Neurosci. 17:7351-8, 1997) these results indicate that Narp is localized to excitatory synapses.

The near complete colocalization of Narp and GluR1 on these aspiny neurons was seen at both early and late time points in culture. Moreover the colocalization of Narp with GluR1 was also seen when the anti-Narp antibody was applied to live cultures, implying this is a cell surface phenomenon. Strikingly, the vast majority of spiny, pyramidal neurons had almost no Narp localized to GluR1 clusters in either fixed or live preparations, although bright Narp immunostaining was present in the cell body. A few (less than 5%), scattered spiny neurons did have detectable Narp immunostaining in their spines. Clues to the origin of these latter cells include their large proximal spines and the fact that they were only seen when the oldest postnatal rats (P6-7) were used as a source of cells. Both observations suggest an association with the dentate gyrus. Whether the distinct distribution of Narp in these subtypes of hippocampal neurons is the result of differential synthesis or subcellular transport is, as yet, unresolved.

In addition to the synaptic localization of Narp, many hippocampal axons, identified by Tau immunostaining, were positive for Narp at early time points in culture (45 of 101 on Day 7 in vitro). These axons are likely to be exclusively excitatory, as GAD positive processes were uniformly Narp negative. Axonal staining was only seen in permeablized preparations. Narp staining, both live and permeablized, was blocked by preabsortion of the Narp antibody with antigen, confirming its specificity.

In cultured spinal neurons, which are almost all aspiny, clusters of Narp immunostaining were more widespread, and were seen to co-localize with the AMPA receptor subunit GluR1 in most cases. Surface Narp immunostaining was seen to colocalize with synaptic GluR1 immunostaining but was not present in synapses devoid of GluR1, which we have previously shown to be inhibitory synapses (O'Brien et al., supra, 1997). Narp clustering at excitatory synapses was also observed when the Narp antibody was applied to fixed and permeablized preparations. Overall 73% (529/720) of GluR1 clusters had associated Narp immunostaining, both in live and fixed preparations. In contrast, surface Narp rarely co-localized with inhibitory synapses (12/218) identified by presynaptic GAD immunostaining. As in hippocampal cultures, Narp immunostaining was also observed in a subset of Tau immunopositive axons at early time points, although its appearance was more clumped than that seen in hippocampal axons. Of note, surface staining with the Narp antibody in early spinal cultures frequently revealed, a few scattered, non-synaptic, dendritic, Narp clusters, which, in contrast to synaptic Narp clusters, were only variably associated with GluR1).

### EXAMPLE 2

### Transfected Narp Accumulates at Excitatory Synapses

In order to characterize the synaptic targeting of Narp in neurons, a C-terminal myc-tagged version of Narp (designated myc-Narp) was transfected into cultured spinal neurons (Dong et al., Nature 386:279-84, 1997). After 72 hours of expression, live staining with an anti-myc (mouse) antibody, followed by a FITC labeled anti-mouse secondary, was used to reveal the surface distribution of the transfected Narp. Subsequent staining of the same cells, after fixation and permeabilization, with an AMCA labeled anti-myc (rabbit) antibody was used to identify transfected neurons and their axons and dendrites, while GluR1 and GAD staining were used to identify excitatory and inhibitory synapses, respectively.

Staining of transfected neurons indicated that myc-Narp, similar to endogenous Narp, was distributed throughout the somato-dendritic domain of the transfected neuron and was also specifically localized to excitatory synapses. In a series of 3 separate experiments, 74% (123/167) of GluR1 clusters on 34 transfected neurons had clear clusters of surface myc-Narp stain. Like in untransfected neurons, nearly all GluR1 clusters in transfected neurons are synaptic (i.e., associated with synaptophysin or synapsin 1 staining). Conversely, myc-Narp staining in transfected neurons rarely (18/114) co-localized with presynaptic GAD staining. In addition to the clusters of transfected Narp associated with GluR1, a slightly smaller number of myc-Narp clusters were also seen that were not associated with GluR1. It is likely that these are similar to the extrasynapic Narp clusters see in non transfected cells. Whether these represent Narp in the process of migrating to excitatory synapses, or represent a form of Narp available to interact with nascent excitatory terminals is yet to be determined.

To exclude the possibility that the live staining technique induced the aggregation of myc-Narp at excitatory synapses, the co-localization of myc-Narp with GluR1 was examined in neurons fixed before staining. In these transfected neurons, 86% (266/309) of the dendritic GluR1 clusters were associated with focally clustered myc-Narp staining.

### EXAMPLE 3

### Synaptic Narp is Derived from Both the Presynaptic and Postsynaptic Neuron

Since only 1 to 3% of the neurons in a given experiment are transfected by the procedure described above, it was relatively easy to distinguish myc-Narp that came from the postsynaptic cell (surface stain associated with the large proximal processes of an isolated transfected cell) from myc-Narp originating from the presynaptic cell (surface stain associated with a solitary thin process coursing over a non-transfected cell body or dendrite which is not in continuity with any visibly transfected cell). This operationally defined distinction between axon and dendrite was verified to be nearly 90% specific in pilot experiments in which labeled processes were stained with anti-Tau antibodies to distinguish axons from dendrites. As discussed above, 74% of excitatory synapses in transfected neurons had clear surface myc-Narp stain, indicating that Narp is targeted to the synapse from the postsynaptic neuron. However, additional observations suggested that Narp was also targeted to the synapse from the presynaptic terminal. Myc-Narp positive processes were frequently observed far removed from any transfected neuronal cell bodies (autaptic connections between Narp positive axons and dendrites were uncommon). Parallel studies revealed that these processes were axons, as 80 of 91 such processes were strongly positive for the axon specific protein Tau. Nearly all the myc-Narp containing axons were immunonegative for GAD, suggesting that myc-Narp, like endogenous Narp, is not transported down the axons of GABAergic neurons. Thus, presynaptic myc-Narp was largely restricted to axons of excitatory neurons. This pattern of axonal staining is reminiscent of the endogenous Narp positive axons seen in early spinal arid hippocampal cultures. When myc-Narp containing axons contacted a GluR1 positive, non-transfected neuron, 37 of 48 such contacts were associated with at least one GluR1 cluster. Clusters were invariably seen at sites where live myc-Narp staining revealed extracellular myc-Narp (for example, in one microscopic field 54 myc-Narp clumps were associated with 51 GluR1 clusters). These observations indicate that the myc-Narp transgene is derived from the presynaptic element.

Electron microscopy from rat hippocampus confirmed and extended many of the observations made with light microscopy in vivo and in vitro. Immunogold labeling was found in presynaptic terminals, and was especially prevalent in mossy terminals of the hilus and CA3 stratum lucidum, although labeling was also seen in other areas of the hippocampus. Presynaptic gold was often seen in the region of synaptic vesicles. Gold also was found in synaptic clefts and postsynaptic densities. In general, postsynaptic labeling was not as dense as presynaptic labeling. In controls in which the primary antibody was preadsorbed with fusion protein, gold was very uncommon in presynaptic or postsynaptic structure; and was absent in the synaptic cleft. The surprising occurrence of pre and postsynaptic Narp in spines of the dentate gyms in vivo contrasts with results in vitro where spiny Narp staining was decidedly uncommon. One possible explanation for this discrepancy lies in the postnatal development of the dentate gryus (Bayer and Altman, J. Comp. Neurol. 158:55-79, 1974) where much of the spiny Narp immunostaining was seen in vivo. These cells would be under represented in cultures taken from perinatal animals. Alternatively, the relative sensitivities of the two techniques may differ.

### EXAMPLE 4

### Over Expression of Narp Increases the Number of Excitatory Synapses in Cultured Spinal Neurons

Given the presence of Narp on both pre and postsynaptic processes and the biochemical propensity of pentraxins to form head to head multimeric aggregates (Emsley et al., Nature 367, 338-45, 1994; Gewurz et al., Curr. Opin. Immunol. 7:54-64, 1995; Bottazzi et al., J. Biol. Chem. 272:32817-23, 1997), the possibility that Narp may function to facilitate the formation of excitatory synapses was explored. Thus, the number of synaptic GluR1 clusters (i.e., GluR1 clusters associated with synaptophysin staining) in transfected neurons over expressing myc-Narp was compared with the number of synaptic GluR1 clusters in untransfected neurons or in neurons transfected with a control plasmid containing the C-terminus of the NMDA receptor subunit NR1A (NR1CT).

In five transfection experiments, the number of synaptic GluR1 clusters on Narp transfected neurons was increased 1.9 fold compared to either control (Table 1; p<0.01). A similar increase in the total number of synaptophysin clusters (synapses) per neuron was observed in Narp transfected neurons. No change was noted in the number of GAD positive terminals on Narp transfected neurons or in the size of the visualized neuritic field of Narp transfected neurons. Interestingly, no increase in non-synaptic GluR1 clusters was noted.

TABLE 1
The effect of NARP overexpression on cultured spinal neurons.

| | NARP Transfected | | | | NR1CT Transfected | | | |
|---|---|---|---|---|---|---|---|---|
| | Transfected | | Untransfected | | Transfected | | Untransfected | |
| Synaptic GluR1 Clusters | 13.0 +/− 3.8 ** | (n = 59) | 7.4 +/− 1.2 | (n = 77) | 6.9 +/− 1.4 | (n = 51) | 6.1 +/− 1.3 | (n = 71) |
| Synaptophysin Clusters | 16.0 +/− 4.6 * | (n = 59) | 11.8 +/− 1.2 | (n = 77) | 10.9 +/− 2.3 | (n = 51) | 10.5 +/− 1.6 | (n = 71) |
| Neuritic Length (Tm) | 225 +/− 51 | (n = 32) | — | | 220 +/− 60 | (n = 33) | — | |
| GAD Clusters | 5.9 +/− 3.9 | (n = 40) | 5.4 +/− 2.8 | (n = 37) | 6.3 +/− 3.2 | (n = 33) | 6.0 +/− 2.1 | (n = 43) |

Cultured spinal neurons were transfected with either a myc-tagged NARP or NR1CT construct as described in Methods. The number of synaptic GluR1 clusters as well as the number of total synapses were calculated in transfected and non-transfected neurons in each condition in a series of 5 experiments. The calculation of neuritic length was taken from a subset of 3 of the 5 experiments and was determined using the permeablized myc staining at 100X (No attempt was made to distinguish axons from dendrites or to measure the entire neuritic length). In a separate series of 3 experiments, the number of GAD clusters on similarly transfected neurons were calculated. Numbers are expressed as +/− S.D of the mean for each experiment.
** P < .01 (paired) for all comparisons.
* P < .05 (paired) for all comparisons.

### EXAMPLE 5

### Narp Clusters the AMPA Receptor Subunits GluR1-3 in Transfected 293 Cells

The clustering of AMPA receptors is a major characteristic of excitatory synapse formation. To investigate the possible role of Narp in AMPA receptor clustering (similar to rapsyn aggregating nicotinic acetylcholine receptors at the neuromuscular junction; see Phillips et al., J. Cell. Biol. 115:1713-23, 1991; Ramarao and Cohen, Proc. Natl. Acad. Sci. USA 3:4007-12, 1998), the ability of Narp to aggregate AMPA receptors was investigated. When expressed alone, Narp (or myc-tagged Narp) forms large surface aggregates which can be seen in live or permeablized preparations. These same surface clusters are also seen when an Fab fragment of the anti-myc antibody is used on live preparations. Moreover, using a marker for transfected cells, surface Narp staining was only seen in transfected cells, and was not the result of non-specific binding of secreted protein to untransfected cells. In contrast to Narp, GluR1 is normally distributed diffusely either in live or fixed preparations.

Co-expression of GluR1 with Narp induced GluR1 to form large surface aggregates which co-localized with the Narp clusters. These Narp-GluR1 aggregates were seen either with surface staining using whole antibody or Fab fragments, or in fixed and permeablized preparations. In addition to GluR1, Narp induced aggregates of GluR2 and GluR3 but not GluR4, GluR6, NR1, NR1/2A or the neuronal glutamate transporter EAAC1. Of note, preliminary electrophysiological studies in transfected 293 cells showed no differences in AMPA receptor current amplitudes or desensitization kinetics in the presence or absence of co-expressed Narp.

### EXAMPLE 6

### Narp Co-Immunoprecipitates with AMPA Receptor Subunits in Both 293 Cells and Brain

Co-immunoprecipitation experiments were used to examine whether Narp is physically associated with AMPA receptors in HEK-293 cells. Immunoprecipitation of Narp demonstrated that Narp was specifically associated with the GluR1, GluR2 and GluR3 subunits in co-transfected cells. Interestingly, treatment of transfected cells with 1 mM tunicamycin had no effect on the association between Narp and any of the AMPA receptor subunits, implying that the lectin property of Narp (Tsui et al., supra, 1996) does not mediate its interaction with AMPA receptor subunits.

To determine whether Narp is associated with AMPA receptors in vivo, co-immunoprecipitation experiments were performed from rat brain. As in the HEK cells, Narp was found to be specifically associated with GluR1 when either anti-Narp or anti-GluR1 antibodies were used to isolate the complex. The co-immunoprecipitation of Narp and GluR1 was blocked by preincubating the initial antibody with its cognate peptide, and no immunoprecipitation was seen with preimmune serum. These studies suggest that Narp and GluR1-3 could directly interact. Furthermore, their association in 293 cells indicates that other neural-specific proteins are not required for these interactions to occur.

### EXAMPLE 7

### Narp Induces the Aggregation of AMPA Receptor Subunits in Apposing Cells

To evaluate whether Narp could mediate intercellular aggregation of GluR1, similar to that expected at a synapse, myc-tagged Narp and GluR1 were expressed separately in 293 cells and subsequently co-cultured together. Under these conditions, surface Narp clusters on one cell could induce large surface GluR1 aggregates on another. In a total of 109 contacts between a GluR1 expressing transfected cell and a Narp expressing cell, there was a mean of 1.2+/−0.75 large overlapping GluR1/myc-Narp clusters at these sites of contact. The specificity of the interaction between GluR1 and Narp was evidenced by the fact that Narp did not induce clusters of either the neuronal glutamate transporter EAAC1 (0.04+/−0.05; n=121) or the NMDA receptor subunits NR1 and NR2A (0 coclusters; n=32 contacts). Moreover, the number of GluR1 clusters at sites of contact with non-Narp expressing 293 cells was extremely low [0.07 R1 clusters per contact with a GluR2 expressing cell (n=76); 0.03 R1 clusters per contact with a Pick1/GluR2 expressing cell (n=58)]. The interaction between GluR1 and Narp required contact between the heterologous cells, as there was no evidence that Narp could diffuse from one cell and induce GluR1 clusters on another.

These results indicate that Narp and GluR1 do not need to be expressed in the same cell for AMPA receptor aggregation to occur. However, when untagged Narp was co-expressed with GluR1 and then co-cultured with cells expressing only myc-tagged Narp, there was a greater than two fold increase in the number of intercellular myc-Narp*GluR1 co-clusters (mean 2.6+/−1.4 n=124; p<0.01). This suggests that the expression of Narp in both the “pre-” and “postsynaptic” cell facilitates cluster formation. The effect of co-expressed Narp on the incidence of intercellular Narp-GluR1 clusters occurred in the absence of any effect on the expression/accumulation of GluR1. To ensure that the surface staining technique did not induce the intercellular co-clustering of GluR1 and Narp, we examined the co-localization of GluR1 and Narp in cells fixed and permeablized prior to staining, as well as in cells in which live staining was done with an Fab fragment to prevent antibody induced aggregation. In all cases the results were identical, both for the ability of Narp to cluster AMPA receptors on apposing cells, and for the coexpression of Narp with GluR1 in the “postsynaptic” cell to potentiate this process.

### EXAMPLE 8

### Narp Clusters GluR1 in Cultured Spinal Neurons

Although Narp is secreted from transfected 293 cells and can be collected from the media, it appeared to have no bioactivity in this form, assayed by its ability to bind to other 293 cells transfected with Narp and/or GluR1. Moreover this soluble form of Narp did not bind to cultured spinal neurons and did not cluster glutamate receptors on these cells. However, the bioactivity of Narp could be readily demonstrated when 293 cells expressing myc-Narp on their surface were mixed with spinal neurons previously grown in culture for 4 days. The neurons and transfected HEK-293 cells were cocultured for 48 hours and then fixed and stained for myc-Narp, GluR1 and synapsin. When contacts between myc-Narp expressing 293 cells and GluR1 expressing spinal neurons were examined for overlapping myc-Narp/GluR1 clusters, 64% (82/128) of the myc-Narp clusters in contact with the neuron overlapped with neuronal GluR1 clusters (mean of 2.6+/−1.5 overlapping myc-Narp/GluR1 clusters per 293-neuron contact).

Only 5 of the 82 GluR1 clusters associated with myc-Narp had any staining for the synaptic vesicle protein synapsin 1 compared with native GluR1 clusters which almost always (90%) co-localize with synapsin (146/163 on day 6). These results strongly suggest that the myc-Narp transfected 293 cells induced the non-synaptic GluR1 clusters rather than associated with established synaptic GluR1 clusters. To control for random GluR1 clusters which might occur at sites of contact between 293 cells and spinal neurons, 293 cells were transfected with a combination of Pick1 and GluR2, which also form large aggregates on the surface of 293 cells (Xia et al., Neuron 22:179-187, 1999). No clusters of neuronal GluR1 were seen associated with these Pick1/GluR2 clusters at a total of 58 cell-cell contacts involving 88 Pick1 clusters. Moreover there was no evidence that contact with an untransfected 293 cell or a 293 cell transfected with a diffusely expressed construct (such as GluR2) could cluster GluR1 on the contacted dendrite. As a sign of specificity, Narp transfected 293 cells did not cluster the NMDA receptor subunit NR1 or the neuronal glutamate transporter EAAC1. In addition to the typical overlapping clusters of myc-Narp and GluR1 at contact points between neurons and 293 cells, clusters of myc-Narp which appeared to have broken off from processes of 293 cells were also capable of clustering GluR1. Although the number of these latter aggregates were not formally tabulated, they appeared to be at least as numerous as those still obviously in contact with the 293 cell. We did not see an ingrowth of axons into the transfected 293 cells. The reasons for this discrepancy is not certain but it is possible that axons avoided the 293 cells (transfected or not).

Thus, it has been determined that the neuronal IEG Narp is selectively expressed at the majority of excitatory, axo-dendritic shaft synapses on (aspiny spinal cord and hippocampal neurons in vitro. In addition, a small number of spine bearing neurons express Narp at their excitatory synapses in culture. In vivo, immunoelectronmicroscopy confirms Narp to be present at both pre- and postsynaptic sites of spiny and aspiny synapses. The prominent presynaptic localization of Narp in mossy fiber terminals is associated with synaptic vesicles. It has been further demonstrated that Narp is capable of clustering AMPA receptors and that this clustering activity involves a physical interaction (direct or indirect) with AMPA receptor subunits. Because Narp is dramatically upregulated in neurons in response to patterned synaptic activity and is expressed at relatively high levels in developing and adult brain (Tsui et al., supra, 1996), the studies suggest that Narp may play an critical role in linking activity with the Development of plasticity and excitatory synapses.

The predominant expression of Narp at axo-dendritic shaft synapses in vitro infers highly specific cellular expression and subcellular targeting. Narp appears to be targeted to synapses from both the pre- and postsynaptic cell. Evidence for specific presynaptic localization is provided both by electron microscopy (EM) and by the observation that Narp is expressed in a subset of axons in both native and transfected cells. Strong endogenous Narp staining is seen in axons in both hippocampal and spinal neurons. The fact that none of these axons show staining for GAD, a marker for GABAergic interneurons which comprise approximately 25% of the neurons in spinal cultures (O'Brien et al., 1997) and 30% of the neurons in these postnatal hippocampal cultures, suggests that Narp is transported only into the axons of glutamatergic neurons. Furthermore, in transfected neurons, epitope tagged Narp is only seen in GAD negative axons where it becomes externalized exclusively at excitatory terminals. Narp is also present on the dendritic surface of native and transfected neurons at both synaptic and non-synaptic sites. The synaptic localizations are most dramatic and are notably specific for excitatory synapses.

The pattern of glutamate receptor clustering in hippocampal interneurons in vitro appears somewhat similar to that observed in spinal neurons, and different from spiny pyramidal cells. The fact that a molecular difference may exist in the aggregating molecules expressed at excitatory shaft and spine synapses is not surprising. In cultured hippocampal neurons, pyramidal cell spine synapses are highly enriched in NMDA receptors, in contrast to dendritic shaft synapses on aspiny interneurons and spinal neurons, which are more heavily weighed towards AMPA receptors (O'Brien et al., supra, 1997; Rao et al., J. Neurosci. 18:1217-29, 1998; Liao et al., Nature Neuroscience 2, 3743, 1999). In addition, the actin cytoskeleton (Alkon et al., J. Neurosci. 18:2423-36, 1998), the distribution of the Rho target citron (Zhang et al., J. Neurosci. 19:96-108, 1999), and the modulation of AMPA receptors by activity and BDNF (Rutherford et al., Neuron 2:521-30, 1998), all appear different in spiny and aspiny hippocampal neurons. Interestingly, the major excitatory input onto hippocampal interneurons is through the granule cells of the dentate gyrus (Accsady et al., J. Neurosci. 18:3386-3403, 1998) the region most enriched in Narp protein and mRNA (Tsui et al., supra, 1996). Sprouting and synaptogenesis are documented to occur in terminal mossy fibers following seizure (Cavazos et al., J. Neurosci. 11:2795-803, 1991) and may involve the activity of molecules such as Narp which are strongly induced by seizures. In vivo, the distribution of Narp by immuno-electron microscopy appears to be more widespread amongst excitatory synapses than our data in vitro would indicate, with spine as well as shaft accumulation.

The family of long pentraxins, of which Narp is a member, has several characteristics which might play a role in promoting excitatory synapse formation. Included among these are the ability to form side to side and head to head multimeric aggregates (Bottazzi et al., J. Biol. Chem. 272: 32817-23, 1997; Emsley et al., Nature 367, 338-45, 1994; Goodman et al., Cytokine Growth Factor Rev. 2: 191-202, 1999, and the ability to bind other proteins via a lectin like domain. When assayed by non-reducing SDS-PAGE, native Narp migrates with an apparent size of >200 kDa and this mobility shifts to ˜55 kDa with addition of reducing agent, consistent with the prediction that Narp forms cysteine-linked multimers. These multimers however are far below the size of the large macroaggregates seen on the surface of transfected cells, a property unique to Narp among the family of pentraxins. It is possible that an additional domain in the unique N-terminus mediates the formation of these macroaggregates. The ability of Narp to cluster AMPA receptors would not have been predicted from a knowledge of the family of pentraxins, since the association of Narp with AMPA receptor subunits in the presence of tunicamycin suggests that it is not the lectin component of Narp which mediates this interaction. Indeed, the specificity of the interaction (GluR1-3 but not GluR4, 6, and NR1) would also argue against a non-specific interaction such as that mediated by a lectin.

Given the localization of Narp at excitatory synapses, its AMPA clustering activity may play an important role in the synaptic aggregation of those receptors. Biochemical and immunohistochemical characterization demonstrates the specificity of the interaction between Narp and AMPA receptor subunits. When viewed in the context of other glutamate receptor clustering molecules, Narp displays several novel features. In contrast to the intracellular proteins PSD-95 and GRIP, Narp is an extracellular molecule with no PDZ domains and no access to intracellular domains on AMPA receptor subunits. These results suggest that if Narp directly interacts with AMPA receptor subunits, it interacts with extracellular domains on these proteins. Narp's ability to form large aggregates contrasts with the pattern seen with PSD-95, which by itself is diffusely expressed (Kim et al., 1996). Like rapsyn (Ramarao and Cohen, 1998) and Pick1 (Xia et al., 1999), Narp has coiled-coil domains in its unique N-terminus (Tsui et al., 1996), which may be available for interaction with extracellular components of GluR1-3 as well as for Narp-Narp interactions. It is very likely that Narp directly interacts with AMPA receptor subunits. Although unlikely, the presence of an additional linker molecule in both neurons and HEK-293 cells has not been completely ruled out.

Another notable functional property of Narp expressing cells is their ability to cluster AMPA receptors on apposing cells, even when the contacted cell does not express Narp. In view of the documented physical interaction between Narp and AMPA receptors when these proteins are expressed in the same cells, it seems likely that the intercellular clustering activity also involves their physical interaction. The transcellular clustering activity of Narp is further enhanced when the apposing cell co-expresses Narp with AMPA receptors, suggesting that a Narp-Narp interaction may also contribute to transcellular clustering. In this regard, Narp may potentially display similarities with cadherins, which self associate and participate in synaptogenesis from both the pre and postsynaptic surfaces (Fannon and Colman, Neuron 17:423-34, 1996; Uchida et al., J. Cell. Biol. 135:767-79, 1996). Unlike the family of cadherins, however, Narp appears to be completely extracellular with no transmembrane domain (Tsui et al., supra, 1996). Moreover, direct proof that Narp functions as an adhesion molecule is lacking. The hypothesized function of Narp to transynaptically aggregate AMPA receptors is unique but reconcilable when considering that synaptic distances in the CNS are small due to the lack of a well defined basement membrane (Gordon-Weeks et al., Exp. Physiol. 77:68 1-92, 1992), and that Narp is likely to be secreted and may bind postsynaptic proteins at an intermediary point between the two cells. EM localization shows Narp in the synaptic cleft, and are consistent with this proposed function.

A model in which Narp-Narp interactions between pre- and postsynaptic cells contribute to excitatory synapse formation with a secondary, clustering of synaptic AMPA receptors due to Narp-AMPA receptor interactions is proposed. In support of a “presynaptic” effect of Narp, one should note that Narp expressed on heterologous cells induces AMPA receptor clusters on neurons. By contrast, a “postsynaptic” effect of Narp is suggested by the observation that when Narp expression is upregulated by transfection, it results in a two fold increase in the number of excitatory synapses, with no change in the number of inhibitory synapses. Since only a small percentage of cells are transfected in this experiment, the increase in number of excitatory synapses on transfected cells as compared with non-transfected cells in the same dish argues strongly for a postsynaptic action of Narp. The synaptogenic effect of upregulated Narp expression in neurons would be a manifestation of the potentiating effect of Narp on transcellular cluster formation seen in heterologous cells when Narp is co-expressed with AMPA receptor subunits. It is also possible that excess Narp in the postsynaptic neuron promotes Narp-Narp intercellular interactions with presynaptic elements and makes it more favorable for the over expressing cell to attract new synapses. This would favor excitatory synapses over inhibitory synapses, since inhibitory axons do not appear to express Narp. Preliminary observations suggest that the small extrasynaptic clusters of Narp seen in native and transfected cells lack an association with GluR1. It may be that only the large Narp clusters seen at synapses or in 293 cells are able to recruit AMPA receptors, perhaps due to interacting domains on Narp. Further analysis of the structure-function relationship for Narp should help reveal all the mechanisms that contribute to synaptogenesis.

Several attempts have been made to disrupt endogenous synaptogenesis using one anti-Narp antibody. However, the anti-Narp antibody currently available does not appear to be a blocking antibody up to a concentration of 30 mg/ml either in spinal cord cultures or in the 293 cell assay. More antibodies will be developed. Antisense oligonucleotides directed to several sites on Narp have had no effect on Narp production assayed by immunoblots and immunohistochemistry. Other antisense oligonucleotides are currently being studied to produce agents that inhibit Narp expression. Further investigations will also be made into the soluble form of Narp.

Narp provides further evidence that the neuronal IEG response can directly modify synaptic function. Other examples include Homer, which regulates the coupling of synaptic group 1 metabotropic glutamate receptors to inositol trisphosphate receptors (Tu et al., Neuron 21.717-726, 1998), RGS2 which regulates coupling of specific G-protein linked receptors to their down stream signaling cascade (Ingi et al., J. Neurosci. 18:7178-88, 1998), Cox-2, which is the nodal enzyme in prostaglandin synthesis (Kaufmann et al., Proc. Natl. Acad. Sci. USA 93, 2317-21, 1996: Yamagata et al., Neuron 11:371-86, 1993), and Arc (Lyford et al., Neuron 14, 433-45, 1995; Steward et al., Neuron 21:741-51, 1998), which may function as an adapter protein for CaMKII. One of the challenges in describing the contribution of IEGs to long-term synaptic plasticity is to understand how a genomic response, that is temporally and spatially remote from an “activated” synapse, can selectively modify the function of specific synapses. Emerging evidence suggests that IEG products may be targeted to specific active synapses. For example, Arc mRNA selectively accumulates at dendritic sites of recent synaptic activity (Steward et al., Neuron 21:741-51, 1998). The present studies suggest an alternative mechanism that could mediate synapse-specific effects of Narp. Presynaptic secretion of Narp at active synapses could target its action to specific synapses where it could cluster, or perhaps stabilize, GluRs and thereby selectively strengthen active synapses. Thus Narp provides new tools to study the mechanisms of synapse-specific and protein synthesis-dependent neuronal plasticity.

### EXAMPLE 9

### Neural Activity Regulated Pentraxin Binds the AMPA Receptor X-Domain and Mediates Synaptic Capture

In this example, the Narp binding site in GluR1 is demonstrated to be within a discrete, 40 amino acid region (Narp Association Region 1, NAR1) of the N-terminal “X domain.” Like wild type GluR1, a modified GluR1 lacking the NAR1 region (GluR1ΔNAR1) trafficks to the membrane with Stargazin, but displays increased diffusional mobility on the dendritic surface of hippocampal neurons. Narp transgene markedly reduces mobility of wild type GluR1, but does not alter diffusion properties of GluR1ΔNAR1. Deletion of NAR1 from GluR1 results in inefficient targeting to synapses, while insertion of NAR1 into the X-domain of GluR6 confers anomalous synaptic targeting. These studies identify a novel molecular interaction that mediates diffusional capture of AMPAR by Narp, and is essential for normal targeting of AMPAR to developing synapses.

Reagents. Polynucleotide sequences encoding the EC1 and EC2 fragments of GluR1 were cloned into the pDisplay vector (Invitrogen) by polymerase chain reaction (PCR) amplification from the plasmid GluR1/pRK5. PCR primers were engineered to contain the appropriate flanking sequences as well as SacII restriction sites for cloning into pDisplay. HA-GluR1 and HA-GluR6 were also generated from PCR amplification products of GluR1/pRK5 and GluR6/pRK5 with PCR primers containing flanking sequences of the mature protein including the 3′ stop codon and SacII restriction sites for subsequent insertion into pDisplay. HA-GluR1 and HA-GluR6 were excised from pDisplay and subcloned into pRK5 to achieve maximal protein expression using an EcoRI/SalI digestion strategy. An HA-GluR1 construct lacking NAR1 (HA-GluR1ΔNAR1) was generated using with a PCR-based site-directed mutagenesis strategy using sense and antisense primers containing a hexa-glycine flanked by sequences adjacent to the deleted region and using HA-GluR1 as the template. HA-GluR1ΔNAR2 (i.e., an HA-GluR1 construct lacking NAR2) was generated using the same strategy. HA-GluR1ΔNAR1,2 (i.e., an HA-GluR1 construct lacking both NAR1 and NAR2) was made using the same primers used for the creation of HA-GluR1ΔNAR2, with HA-GluR1ΔNAR1 as the template. HA-GluR1 and HA-GluR1ΔNAR1 were digested with EcoRI and SalI and subcloned into plasmid pIRES2 EGFP (BD Biosciences Clontech). HA-GluR6+NAR1 (i.e., an HA-GluR6 construct in which the corresponding GluR6 sequence has been replaced with NAR1) was generated using a megaprimer method in which NAR1 was PCR amplified from GluR1 with the addition of homologous flanking GluR6 regions in the primers. The resulting “megaprimer” PCR product was used and primed against HA-GluR6 template for replacement of the corresponding GluR6 sequence with NAR1.

HEK 293T transfection and co-immunoprecipitation. Confluent 10 cm dishes of HEK 293T cells were split 1:6 into 6-well tissue culture dishes containing culture media (Dulbecco's Modified Eagle Medium with Glutamax, 10% Fetal Bovine Serum, Invitrogen). After 12-24 hours of incubation at 37° C., 5% CO₂, cells were transfected with the appropriate plasmid construct using Fugene 6 (Roche) according to the manufacturer's protocol. After 24 hours of incubation at 37° C., 5% CO₂, cells were lysed in RIPA buffer (50 mM Tris-HCl, 150 mM NaCl, 0.5% Sodium-Desoxycholate, 1% NP-40, 0.1% SDS, 1 mM PMSF, “Complete” Protease Inhibitor Cocktail (Roche), pH 7.4) for 10 minutes on ice. Un-solubilized cellular debris was removed by 10 minute centrifugation at 13,000 rpm at 4° C. A small aliquot was removed for analysis as input while the remaining solution was incubated with 2 μL of rabbit polyclonal Narp antiserum rocking at 4° C. for 1 hour. After adding 25 μL of pre-washed GammaBind G sepharose beads (AP Biotech), samples were rocked at 4° C. for an additional 45 minutes. The samples were then washed 2 times with HNTG Buffer (10% Glycerol, 1% Triton X-100, 150 mM NaCl, 20 mM Hepes pH 7.4), 2 times with HNTG Buffer+500 mM NaCl, and 2 times with HNTG Buffer. Samples were diluted in sample buffer and boiled for 1 minute before standard SDS-PAGE analysis. Proteins were visualized with western blotting using mouse monoclonal HA antibodies (Santa Cruz) and peptide-generated rabbit polyclonal Narp antisera directed against the C-terminal region.

Cos-7 cell transfection and surface staining. Cos-7 cells were selected for immunostaining experiments because their relative flatness facilitates imaging. Confluent plates of Cos-7 cells were split 1:10 into 12-well dishes containing pre-coated coverslips (Carolina Biological) in culture media. Coverslips were prepared by autoclaving, followed by a 15-minute incubation with 2% gelatin, and PBS washing. After 12-24 hours of incubation at 37° C., 5% CO₂, cells were transfected with the appropriate plasmid construct using Fugene 6 (Roche) according to manufacturer's protocol. After 24 hours of incubation in 37° C., 5% CO₂, the coverslips were incubated in serum free culture media with primary antibodies for 15 minutes at room temperature. The primary antibodies were used at a dilution of 1:1000 for the mouse monoclonal HA antibody (Santa Cruz) and 1:200 for the rabbit polyclonal Narp antiserum. The coverslips were then washed with DMEM, fixed, blocked in 10% Fetal Bovine Serum (FBS)/PBS for 1 hour at room temperature, incubated with fluorescence conjugated secondary antibodies (Jackson Laboratories) diluted 1:500 in blocking solution for 1 hour at room temperature, washed in PBS, and mounted in ProLong Antifade Reagent (Molecular Probes).

In Vitro Binding. Polynucleotides encoding NAR fragments were subcloned into pGEX-4T-2 vector (Amersham) in the EcoRI and SalI sites. The constructs were confirmed by restriction enzyme digestion, DNA sequencing and fusion protein expression. GST-fusion proteins were purified by bacterial inoculation of one clone containing GST-fusion protein plasmid into 5 mL of LB liquid medium and cultured at 37 C overnight. Cultured bacteria was transferred into 400 mL of LB liquid medium and cultured at 30 C for 3 hours. Protein expression was induced with 5 mM IPTG and the bacteria were cultured at 30 C for another 2 hours. A bacterial pellet was collected and resuspended in 10 mL of lysis buffer (pH 7.4 PBS containing 10 mM sodium pyrophosphate, 1 mM sodium orthovanadate, 1% triton and 0.1% PMSF), sonicated for 10 seconds, 3 times at 4 C. Debris was pelleted with centrifugation at 12,000 rpm at 4 C for 10 minutes. The supernatant was incubated with Glutathione Sepharose 4B beads (Pharmacia) with agitation at 4 C for 2 hours. Beads were washed with lysis buffer+1% triton+0.1% PMSF twice; lysis buffer+PMSF twice; PBS+0.5M NaCl once; and PBS twice. The fusion protein bound beads were then used in the in vitro binding assay. Purified Narp protein preparation was accomplished by splitting 293T cells is into 100 mm culture dishes at the density of 5×10⁵ cells/mL. Cells were transfected with myc-Narp in pRK5 with Lipofectamine (Invitrogen) 24 hours after splitting. Transfected cells were harvested 48 hours later. The cells were pelleted, re-suspended and sonicated in 5 mL lysis buffer with 1% triton+0.1% PMSF at 4° C. Insoluble debris was pelleted and discarded by centrifugation at 10,000 rpm for 10 min. The supernatant used as the Narp protein supply for in vitro binding. GST-fusion protein bound glutathione-conjugated beads was incubated with purified Narp at 4° C. with rotation for 2 hrs. Beads were washed with lysis buffer+0.5% Triton X-100; lysis buffer+0.25% Triton X-100; twice with lysis buffer alone; PBS+5M NaCl; and twice with PBS alone. Co-immunoprecipitation was visualized by standard SDS-PAGE.

Neuronal transfection and immunostaining. Cortical neurons were incubated in 1 mL of pre-equilibrated Neurobasal Media (Invitrogen). 3 μg DNA was mixed with Neurobasal Media, and 3 μl Lipofectamine 2000 (Invitrogen). Lipofectamine complexes were incubated at RT for 10 minutes then placed on neuronal coverslips for 1 hour at 37° C., 5% CO₂. Cells were returned to neuronal culture media for 12-24 hours. Surface labeling was accomplished by live application of primary antibody for 30 minutes at 37° C. Neurons were fixed in 4% sucrose, 4% paraformaldehyde and permeabilized in 0.2% Triton X-100. Neurons were blocked in 10% Fetal Bovine Serum (FBS)/PBS for 1 hour at room temperature, and incubated in PBS with primary antibody for 1 hour at room temperature. Neurons were washed with PBS, incubated with fluorescence conjugated secondary antibodies (Jackson Laboratories) diluted 1:500 in blocking solution for 1 hour at room temperature, washed in PBS, and mounted in ProLong Antifade Reagent (Molecular Probes). Primary antibodies were used at a concentration of 200 ng/mL for the mouse monoclonal HA antibody (Santa Cruz), 5 μg/mL for the monoclonal PSD-95 antibody (UBI), 2.5 μg/mL polyclonal Narp antibody (JH3412), and 1:2000 crude polyclonal sera for NP1 (JH3415). Transfection efficiencies raged from 1-5%. Quantitation of immunostaining was done using a computer-assisted image processing (Metamorph).

Single Quantum Dot (QD) Tracking. Hippocampal neurons were prepared from postnatal days 1-3 mouse pups and cultured in Banker Style conditions to 6 D. I. V., 8 D. I. V., or 11 D. I. V. Cultured hippocampal neurons were transfected as above. 0.5 μL of QD 655 mouse IgG (QuantumDot Corporation) was incubated with 0.8 μg of HA antibody (Santa Cruz) in a 20 μL volume of PBS for 1 hour at room temperature. Neurons were incubated in a 1:500 dilution of this QD solution for 15 minutes at 37° C., 5% CO₂. After brief washes with culture media, the neurons were mounted on a live imaging chamber with culture media/10 mM Hepes. Transfected neurons were identified based on GFP fluorescence. 100 millisecond exposure images were collected from neurons at a frequency of 2.67 Hz for 60 seconds with a coolsnap HQ (Photometrix). The graphical and numerical representation of the trajectory of each QD was extracted from each stack of images using the Motion Analysis Package of Metamorph (Universal Imaging Corporation). The numerical representation of the trajectory was used to calculate a diffusion coefficient using MSD software. Analysis was performed blinded.

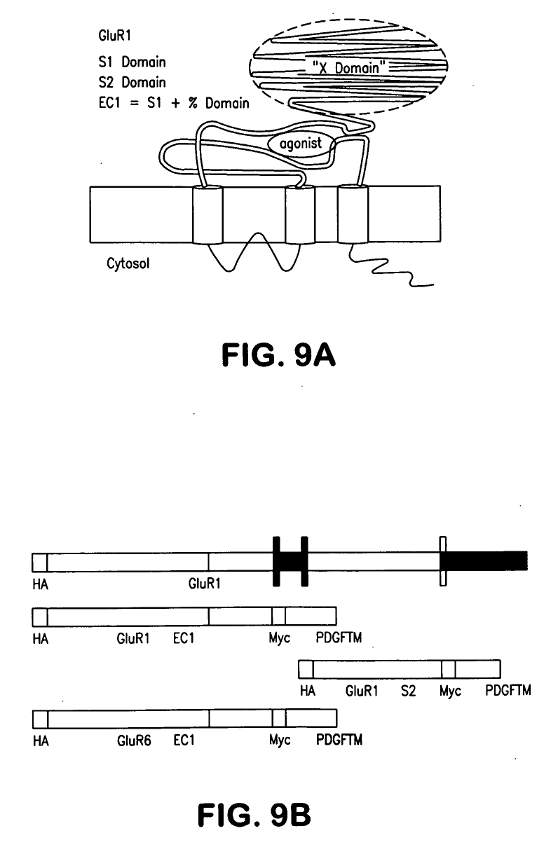

Discrete regions in extracellular domains of GluR1 bind Narp. Structure-function studies of the extracellular domains of AMPARs in the full length receptor have proven challenging since even conservative mutations can result in failure of cell surface expression. Thus, to identify binding sites for NP, fusion constructs were generated that separately display the extracellular domain 1 (EC1) and extracellular domain 2 (S2) of the AMPAR on the surface of the plasma membrane (FIGS. 9A and 9B). When co-expressed with Narp, both EC1 and S2 coimmunoprecipitate (co-IP). By contrast, GluR6, which does not co-cluster with Narp, does not co-IP with Narp. Binding was further demonstrated by the ability of Narp to co-cluster EC1 and S2 domains of GluR1, but not the EC1 domain of GluR6. These data suggest that both extracellular domains of GluR1 can associate with Narp.

Deletion mutants were used to map regions within GluR1 EC1 and S2 domains that are necessary for binding Narp. Deletion of amino acids 1 to 304 (HA GluR1 EC1 AA 305-520 of the GluR1 protein corresponding to NCBI GeneID:2890) of EC1 results in a fragment that does not co-IP with Narp, while a mutant lacking 1-262 (HA GluR1 EC1 AA 263-520) does co-IP. Similarly, deletion of amino acids 618 to 719 (HA GluR1 S2 AA 720-788) of S2 results in a fragment that does not co-IP, while a mutant lacking amino acids 618-676 (HA GluR1 S2 677-788) retains the interaction. These results suggest that amino acids 263-305 and 677-720 are important for GluR1 association with Narp, and are termed Neural Pentraxin Association Region 1 (NAR1) and 2 (NAR2), respectively.

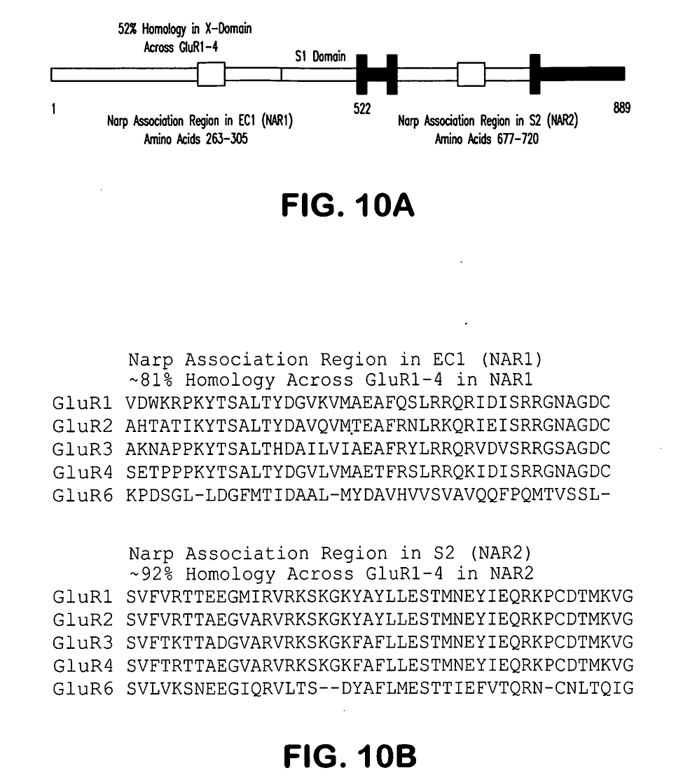

GluR1 with deletions of both NAR1 and NAR2 retains membrane trafficking. To explore the contribution of NAR1/2 to the properties of GluR1, a GluR1 mutant with internal deletions of NAR1 and NAR2 (replaced by polyglycine linkers) was generated. NAR1 and NAR2 are regions of relatively high homology between GluR1,2,3,4 and are distinct from the analogous regions of GluR6 (FIG. 10). and tested to determine whether it could traffic to the plasma membrane when expressed either alone or in combination with Stargazin. Stargazin has been shown to be required for the delivery of AMPA receptors to the plasma membrane of cerebellar granule cells and Xenopus oocytes, and is proposed to function as a chaperone for proper folding and insertion into the plasma membrane. Stargazin interacts with subunits that comprise mature AMPARs (GluR1-4, but not GluR6) and appears to recognize extracellular or transmembrane regions of the receptor. Cos-7 cells were transfected with recombinant receptors tagged with an N-terminal HA tag, and membrane receptor was visualized by labeling the cells live with a monoclonal antibody directed against HA. Surface expression of HA GluR1 increased ˜5 fold in the presence Stargazin. Surface expression of HA GluR1ΔNAR1,2 was less than wild type GluR1 (typically ˜½) but showed a similar increase (˜4 fold) with Stargazin. Stargazin interaction with the NAR mutants is confirmed in biochemical assays by the co-IP of HA-GluR1ΔNAR1 and HA-GluR1ΔNAR2 with myc-Stargazin. Thus, deletion of NAR1 and 2 reduces, but does not destroy trafficking to the plasma membrane and does not interfere with the action of Stargazin.

NAR1 is necessary for co-clustering of GluR1 with Narp, and sufficient to confer co-clustering of GluR6 with Narp. The role of the NAR domains of GluR1 in co-clustering with Narp in heterologous cells was next examined. As reported previously, HA GluR1 co-clusters with Narp on the cell surface of HEK293 cells. By contrast, HA GluR1 ΔNAR1 was detected on the cell surface but did not co-cluster with Narp. HA GluR1ΔNAR2 was also detected on the cell surface but retained the ability to co-cluster with Narp. These results indicate that NAR1 was more important for co-clustering of GluR1 with Narp, and are consistent with the observation that NAR1 binds Narp more avidly than NAR2 (see below). To examine the sufficiency of NAR1 for binding, a chimeric HA GluR6+NAR1 was engineered by substituting NAR1 into the homologous region of GluR6. HA GluR6+NAR1 was expressed on the plasma membrane and co-clustered with Narp, while HA GluR6 did not.

NAR1 mediates binding of GluR1 to Narp. Biochemical assays demonstrate that Narp co-IPs with wildtype GluR1 more effectively than GluRΔNAR1 or ΔNAR1,2 or GluR6. In contrast, insertion of NAR1 into GluR6 conferred binding with Narp. To examine binding in a more defined system, NAR1 and NAR2 were expressed in bacteria as GST fusion proteins and used in pull down assays. myc-Narp from HEK cell lysate bound GST-NAR1 and GST-NAR2, but not a GST fusion protein of the homologous regions in GluR6. NAR1 bound Narp more avidity than did NAR2. These studies support the hypothesis that NPs bind to AMPAR by an interaction that is mediated primarily by NAR1.

Pentraxins are enriched at sites of neurite contact and co-localize with PSD-95. The expression of native, surface expressed Narp and NP1 in dissociated hippocampal culture preparations used for diffusion assays was examined. These cultures, prepared from P1-P3 rat hippocampus, undergo the majority of synapse formation between DIV 7-10. Pentraxin expression was examined at DIV 6, corresponding to the age of transfection in the receptor mobility assay. This DIV revealed early phase synapse formation, reflected by a mixture of diffuse and punctate PSD-95. Both PSD-95 and Narp appeared diffusely expressed along neurites. However, in places where neurites came into contact with one another, both molecules clustered in large co-localized puncta. Neurites showing diffuse expression of Narp formed a distinct subset from those diffusely expressing PSD-95. This reflected the prominent association of Narp with axons, while PSD-95 was present in dendrites. In contrast, NP1 was punctate in DIV 6 cultures and showed extensive co-localization with PSD-95 puncta along neurites. This was consistent with the observation that pentraxins can also be secreted at the dendritic surface. It was concluded that Narp and NP1 are present at developing synapses in these cultures.

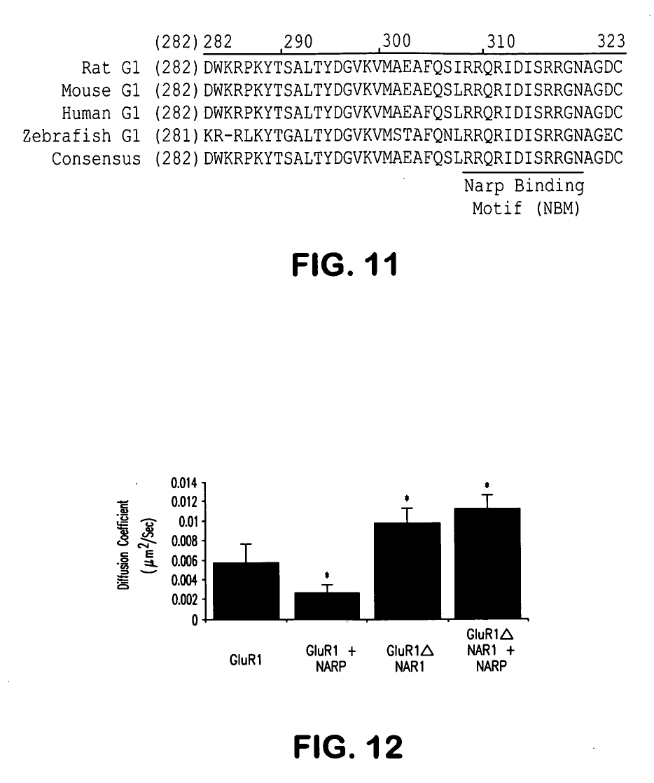

A 12 amino acid sequence within NAR1 is sufficient for Narp binding and predicts a novel N-cadherin/Narp interaction. Analysis of NAR1 sequence revealed a 12 amino acid sequence in NAR1 that is conserved in all AMPAR subunits in all species, and is not present in GluR6 (FIG. 11). It was hypothesized that this conserved 12 aa sequence in AMPAR may be a Narp binding motif. To test this, in vitro binding assays were performed with bacteria GST-tagged GluR1-12mer (GST-G1-12mer) and myc-tagged full length Narp. The equivalent GST-tagged GluR6 NAR1 sequence (GST-G6-Nar1e) was used as control. GST-G1-12mer pulls down Narp, but GST-G6-Nar1e does not. Additionally, a synthetic G1-12mer peptide blocked in vitro binding between GST-G1-NarI and Narp.

Having identified a potential Narp binding motif, other proteins were searched for those that might possess this sequence, and members of the cadherin family were identified. Cadherins function in cell-cell adhesion and are known to play a role in synaptogenesis. The interaction between Narp and one member of the cadherin family, N-cadherin, was tested. Co-IP experiments showed that full-length Narp co-IPs N-cadherin when the proteins are co-expressed in HEK293 cells. As anticipated, the pentraxin domain, which is for AMPAR interaction, is also critical for co-IP of N-cadherin. Immunostaining of COS-7 cells showed that Narp co-clusters on the surface of cells with N-cadherin, and endogenous N-cadherin and Narp form co-clusters in cultured hippocampal neurons. These data support the hypothesis that a 12 aa sequence in NAR1 represents a Narp binding motif that is present in both AMPAR and E- and N-cadherins.

Narp reduces surface diffusion of GluR1 but not GluR1ΔNAR1. GluR1ΔNAR1 was used to examine the hypothesis that Narp and NP1 function to limit the diffusion of receptors, and thereby recruit GluR1 to synapses. As part of a validation of GluR1ΔNAR1 for cell biological studies it was confirmed that it retained agonist-dependent channel activity. This was consistent with the finding that deletion of the entire X-domain did not alter channel activity. The mobility of HA GluR1 relative to HA GluR1ΔNAR1 on the surface of neuronal dendrites were compared using 5 nm fluorescent Quantum Dots (QDs) and Single Quantum Dot Tracking (SQDT). Dissociated hippocampal cultures (DIV 6) were transfected with HA GluR1-IRES2-GFP or HA-GluR1ΔNAR1-IRES2-GFP. QDs were coupled to HA antibodies and applied to living neurons. After washing, neurons were mounted onto a live imaging chamber. QDs adhered specifically to transfected cells, identified by their GFP fluorescence, suggesting specific detection of expressed receptor. QD-HA-GluR1 and QD-HA-GluR1ΔNAR1 were visualized by live, time-lapse fluorescence imaging. The trajectories of QD-coupled receptors were determined by the position of the QD in each frame, and the receptor diffusion coefficients were calculated from QD trajectories over multiple trials. Endogenous Narp expression was confirmed for this preparation. GluR1ΔNAR1 diffused at a higher rate than wt GluR1. At DIV 7, HA GluR1 ΔNAR1 diffused at an average rate nearly twice that of wild type GluR1 (FIG. 12; p<0.05 as compared to HA GluR1 in 2 way ANOVA). Similarly, at 9 D. I. V. HA GluR1 ΔNAR1 diffused nearly 2-fold faster that wt HA GluR1 (p<0.05 as compared to HA GluR1 in 2-way ANOVA). By 12 D. I. V., wildtype HA GluR1 and HA GluR1 ΔNAR1 were not significantly different in their mobility. To test if GluR1 diffusion is modified by Narp, Narp was co-expressed with HA tagged receptors in SQDT assays. At 7 D. I. V., HA GluR1 diffused at an average rate 58% slower than in the absence of Narp transgene (p<0.05 in 2-way ANOVA). In contrast, the HA-GluR1ΔNAR1 average diffusion rate was not significantly altered by Narp transgene. Similar results were obtained at 9 D. I. V. where HA GluR1 diffused 44% slower in the presence of the Narp transgene, while HA GluR1 ΔNAR1 remained unchanged. These observations indicated that the diffusional properties of GluR1 were modified by their ability to interact with Narp during a restricted developmental window.

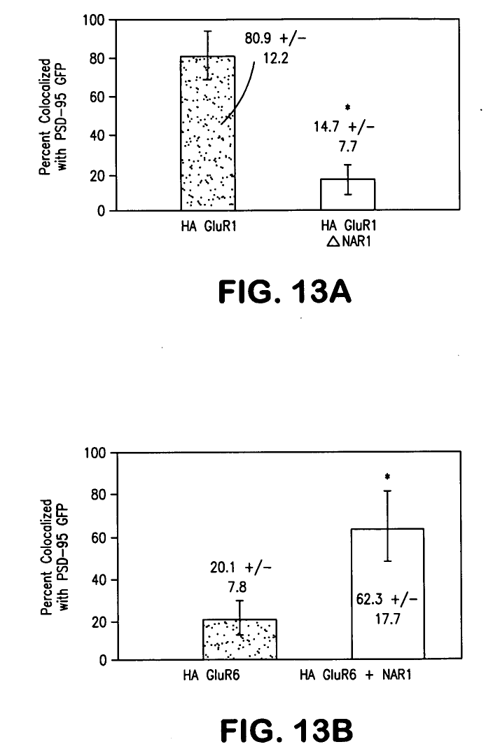

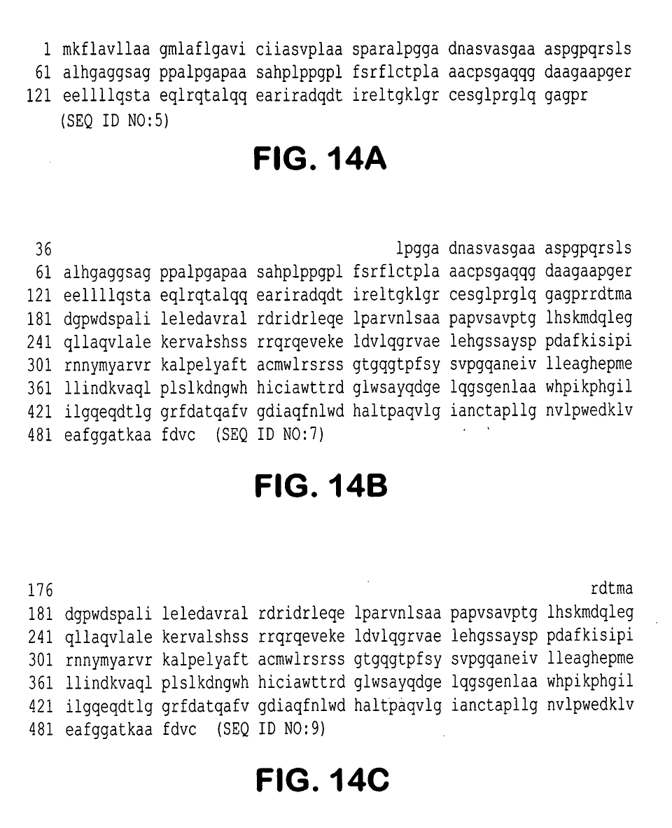

NAR1 is necessary and sufficient for receptor targeting to synapses. To evaluate the role of NAR1 in the synaptic targeting of GluR1, the cellular distribution of HA GluR1ΔNAR mutants was assayed in dissociated hippocampal culture prepared from postnatal day 1 rat pups, transfected at 9 D. I. V. and examined at 10 D. I. V. Neurons were transfected with HA GluR1 or HA GluR1ΔNAR1 together with GFP-PSD-95 as a marker for synaptic sites. The surface population of transfected receptor was detected by live label application of anti HA antibody (Santa Cruz). HA GluR1 showed extensive co-localization with GFP-PSD-95 (FIG. 13A). HA GluR1ΔNAR1 was detected on the neuronal cell surface but did not co-localize with GFP-PSD-95. The preceding observations indicate a necessary role for NAR1 in GluR1 targeting to developing synapses.

To assess whether NAR1 is sufficient to target a membrane glutamate receptor to the synapse, the targeting of GluR6 was compared to GluR6+NAR1. GluR6 was previously reported not to co-localize with the synaptic marker vGlut1, but rather appeared in diffuse non-synaptic puncta along the dendrite. In the present experiments, wildtype HA GluR6 was not enriched at GFP-PSD-95 positive synapses. In contrast, HA GluR6+NAR1 showed extensive co-localization with GFP-PSD-95 (FIG. 13B). Thus, it was concluded that NAR1 is sufficient to confer synaptic targeting of GluR6.

The present experiments identified a discrete extracellular region of GluR1 that is essential for GluR1's interaction with Narp, and for its effective targeting to developing synapses. NAR1 maps to amino acids 262-305 and resides within the “X Domain;” a name that reflects its here-to-fore elusive role in receptor function. NAR1 mediates diffusion-capture of GluR1 during synaptogenesis and is necessary for GluR1 to target developing synapses. Previous studies have shown that complete deletion of the X domain does not alter receptor kinetics, surface trafficking, agonist binding, or potentiation by cyclothiazide in heterologous cells (Pasternack et al., JBC 277:49662-7, 2002). Nevertheless, a small subregion of the X-domain is necessary for GluR1 to target to developing synapses, and when inserted into the X-domain of GluR6 is sufficient to target it to synapses. Moreover, the requirement of NAR1, as demonstrated herein, for biophysical and physiological interactions with Narp provides strong support for the hypothesis that Narp and NP1 mediate interactions in the extracellular compartment that contribute importantly to GluR1 synaptic localization.

The physical association between Narp and GluR1 appears to involve novel structural interactions. The pentraxin domains of Narp and NP1 possess a high degree of sequence similarity and bind AMPARs with similar avidity. The pentraxin domain appears structurally isomorphic to the Laminin G-like (LG or LNS) domain that is present in other synaptogenic proteins like Agrin and Neurexin, despite low primary sequence homology (10-15%). Mutagenesis of the LNS domain of laminin α2 chain defined residues that are critical for interaction with the extracellular subunit of dystroglycan, and distinguished these residues from residues critical for interaction with heparin (Wizemann et al., J Mol Biol 332:635-42, 2003). Crystal structures of the pentraxin domains of C-reactive protein and serum amyloid protein have been reported that identify binding surfaces for interactions with sugars. However, there are no reports of the pentraxin domain interaction with other proteins.

Similarly, there are no empirical data on the structure of the X-domain, but sequence analysis predicts low level homology to bacterial amino acid binding proteins and to the N-terminal extracellular domain of metabotropic glutamate receptors. The X Domain contains the lowest degree of amino acid sequence homology amongst different AMPAR subunits, with only 52-65% homology in pair-wise comparison of rat GluR1 to rat GluR2-4. Interestingly, this homology increases to 81-90% within the NAR1 region (FIG. 10B), and NAR1 of GluR1 is 100% conserved between rat and human and 92% to chicken. It is notable that the X-domain of GluR2 is required for its ability to induce a synaptic spine phenotype in neurons, suggesting a modulatory role in synapse formation. There are no high resolution studies of the X-domain, but sequence analysis predicts low level homology to bacterial amino acid binding proteins, and to the extracellular domain of metabotropic glutamate receptor. The mGluR ligand binding domain has been resolved at 4 Å, and the region corresponding to NAR1 is largely surface exposed, however, the low degree of sequence similarity limits this analysis. Moreover, in our biochemical assays Narp does not bind to mGluR5.

Attempts to identify a conserved motif within NAR1 that is essential for binding to Narp revealed a 12 amino acid sequence that is conserved in all reported AMPAR sequences. This sequence is sufficient to bind Narp, and blocks NAR1 binding to Narp. Moreover, the same putative Pentraxin binding motif was found in members of the cadherin family, and biochemical and histological studies provide evidence that Narp can bind cadherins.

NAR2 is also highly conserved from GluR1-4, and encompasses several residues that are essential for agonist binding. GluR1ΔNAR2 lacks AMPA-dependent channel activity. NAR2 appears to possess less avidity than NAR1 for Narp.

The diffusion limiting property of Narp suggests a molecular mechanism that rationalizes the ability of Narp and NP1 to increase excitatory synapse formation (O'Brien et al., Neuron 23:309-23, 1999). Vertebrate CNS synapse formation occurs en passant, or along the length of the axon at contact sites with developing dendrites. Secretion of Narp into the synaptic cleft of these young synapses may facilitate capture of diffusible AMPARs for synapse formation. The present study supports a role for this mechanism in synaptogenesis since the membrane diffusion of GluR1ΔNAR1 is not restriction by Narp, and GluR1ΔNAR1 does not accumulate at synapses.

Extrasynaptic membrane insertion of GluR1 occurs in physical association with Stargazin, and the AMPAR-Stargazin complex subsequently targets to synapses through capture of its C-terminus by PSD-95. While not wishing to be held to a particular theory, this mechanism may underlie the developmental restriction of the diffusion capture mechanism since both wild type GluR1 and GluR1ΔNAR1 show similar reduced diffusion after DIV12. Nevertheless, the present NAR1-dependent mechanism appears essential for synapse formation and may precede or parallel the Stargazin-PSD95 capture process.

Primary cultures of ganglion neurons from pentraxin knockout mice show delayed maturation of AMPA receptor responses. Moreover, in other studies Narp and NP1 knockout mice show loss of eye specific segregation of projections from the retina to the lateral geniculate nucleus (LGN) without changes in ganglion cell activity. This phenotype is consistent with the present mechanism and predicts that LGN neurons do not establish synapses that are sufficiently functional to mediate effective competition between projections during the developmental critical period. Mechanisms described in models synaptogenesis continue to be relevant for certain forms of plasticity in the adult brain. Narp and NP1 are highly expressed in synaptic regions of adult brain, and Narp is dynamically responsive to physiological synaptic activity.

### EXAMPLE 10

### mGluR1/5-Dependent Long-Term Depression Requires the Regulated Ectodomain Cleavage of NPR by TACE

Pentraxin Expression Constructs. NPR was cloned by screening a lambda zap library of rat hippocampal cDNA. To generate a probe, an NPR polynucleotide was amplified by PCR from a rat hippocampal oligo(dT)-primed cDNA library using the following primers: forward 5′-AAAAAGAATTCCCGGTACCGGCTCTTGCTC (SEQ ID NO:15) and reverse 5′-TCTGGTCTACGAGAAG GAGAAGATCTTTTTT (SEQ ID NO: 16) The PCR product was subcloned into PRK5 using EcoRI and XbaI. A 675 bp NPR fragment within this PCR product generated by EcoRI and NarI restriction enzyme digestion was used as a probe. Of the 29 plaques that were purified and rescued into pBluescript, 24 contained a 4.1 kb cDNA with a 1.5 kb open reading frame encoding NPR; the remaining 5 were not NPR clones. The sequences of two of these clones were matched with Genbank clone NM_{—}030841. There were some discrepancies in the 5′ untranslated region of NPR. However, the veracity of these clones was later confirmed by Genbank NM_{—}030841.2, which replaced NM_{—}030841. The open reading frame of NPR was subcloned into the pRK5 expression vector using the same primers listed above. Constructs with 3′ myc and HA tags were generated using the same forward primer used above and the following reverse primers: 5′CGTTCTCTTCCCGATTCCGTTCTAGATACCTCGTTTTCGAGTAAAGACTCCTT CTAGAGTTAAAGATCT (SEQ ID NO:17) for the myc tag and 5′CTACACACGTTCTCTTCCCGATTCCGTTCTAGATACATAGGTATACTGCAGG GTCTGATACGTATCAGATCT (SEQ ID NO: 18) for the HA tag. The PCR products were subcloned into PRK5 using EcoRI and XbaI. Generation of Narp mutants Narp (C1, 3S)-myc and Narp (C1, 2, 3S)-myc was described previously (Xu et al., Neuron 39:513-28, 2003). Narp (C1, 3S)-myc is a Narp mutant in which cysteines 14 and 79 are mutated to serines. Narp (C1, 2, 3S)-myc is a Narp mutant in which cysteines 14, 26, and 79 are mutated to serines. Chimeras of NP1 and Narp were constructed by introducing silent mutations (resulting in no change at protein level) at specific sites within the cDNAs encoding NP1 and Narp to create unique restriction enzyme sites; chimeras were then created by subcloning digestion products of NP1 and Narp cDNAs into the PRK5 expression vector (Xu et al., supra). The same strategy was employed to construct the Narp/NPR chimeras labeled CM-1 through CM-6. All chimeras were generated to contain C-terminal myc tags. The identities of all constructs generated were confirmed by DNA sequencing.

Antibodies. Three polyclonal antibodies specific for NPR were generated: Ab 4450, Ab 4999, and Ab LFC-NPR. Antibodies 4450 and 4999 were generated by fusing regions of NPR to glutathione S-transferase (GST). Antibody 4450 was generated against a unique region in the second coiled-coil domain of NPR (amino acids 171-287) by amplifying the corresponding region of NPR cDNA using primers: forward 5′GGATCCCGCGACACTATGGCC GACG (SEQ ID NO:19) and reverse 5′GGATGTCAGGGGGTCTACGCAGCTG (SEQ ID NO:20). Antibody 4999 was generated against a region of the first coiled-coil extracellular domain of NPR (amino acids 90-150) by amplifying the corresponding region of NPR cDNA using primers forward 5′GGATCCCTCAGTCG CTTCCTCTGCAC (SEQ ID NO:21) and reverse 5′CTGTGGTAAGCGCTCGAGCAGCTGT (SEQ ID NO:22). The PCR products were subcloned into pGEX-4T2 (Pharmacia, Uppsala, Sweden) using BamHI and SalI. Antibodies 4450 and 4999 were prepared by Covance of Princeton, N.J. in rabbit and guinea pig, respectively, by immunization with purified NPR-GST fusion proteins produced from the above-generated expression vectors using standard protocols. Antibodies were affinity-purified by passing serum through the appropriate NPR-GST fusion protein coupled to AffiGel (Bio-Rad Laboratories, Richmond, Calif.), following the manufacturer's recommendations. Eluted fractions of purified antibody were dialyzed and the titer determined by immunoblot analysis. Cleavage selective antibody to LFC-NPR was generated using strategies previously described (Ye et al., Neuroscience 114:217-27, 2002). Briefly, rabbits were immunized with peptide LPPGTDNASAC (SEQ ID NO:23) and serum collected (Genemed Synthesis, San Francisco, Calif.). The C-terminal cysteine residue was added to facilitate conjugation and to facilitate purification. The antibody was affinity-purified from serum by passing serum through a RAP coupling gel (Zymed, San Francisco, Calif.) to which the peptide was conjugated via the C-terminal cysteine residue. The column was washed and the antibody eluted according to the manufacturer's recommendations. Purified antibody was dialyzed and the titer determined by immunoblot analysis. All other antibodies were purchased commercially or were previously described: PSD-95 antibody (Affinity Bioreagents, #MA1-046, Deerfield, Ill.), β-Actin (Sigma, A5441, St. Louis, Mo.), and EEA1 antibody (Affinity Bioreagents, MA1-5016, Deefield, Ill.). N-terminal GluR1 antibody (JH5072) was a kind gift from Richard L. Huganir.

Cell Culture, Transfection, Western Blot Analysis, and Co-immunoprecipitation analysis. HEK293T and COS-7-7 (ATCC, Manassas, Va.) cell maintenance and transfection methods have been described previously (Xu et al., supra). Preparation and transfection of cultured hippocampal neurons were described previously (Rumbaugh et al., J Neurosci 23:4567-76, 2003). Western blot analysis of homogenates of HEK293T cells, cultured neurons, and brain extracts was performed as described previously (Xu et al., supra). Co-IP analysis to measure interaction between NPR and AMPAR was performed as described (Xu et al., supra).

Immunoelectron Microscopy. Immunogold studies of NPR and LFC-NPR localization were performed as described previously (O'Brien et al., Neuron 23:309-23, 1999; Petralia et al., J Neurosci 13:1722-32, 2001; Petralia and Wenthold, Methods Mol Biol 128:72-93, 1999; Xu et al., supra). Briefly, young adult rats were perfused with 4% paraformaldehyde+0.5% glutaraldehyde and sections of tissue were frozen in liquid propane in a Leica CPC cryopreparation chamber (Vienna, Austria) and then freeze substituted into Lowicryl HM-20 in a Leica AFS. Ultrathin sections on grids were incubated in 0.1% sodium borohydride plus 50 mM glycine in Tris-buffered saline plus 0.1% Triton X-100 (TBST), followed by 10% normal goat serum (NGS) in TBST, then primary antibodies in 1% NGS/TBST, followed by immunogold in 1% NGS/TBST plus 0.5% polyethylene glycol (20,000 MW), and then stained with uranyl acetate and lead citrate. Controls lacking the primary antibody showed only rare gold labeling. Double-labeling included either polyclonal GluR2/3 (Petralia and Wenthold, supra) or monoclonal GluR2 (Chemicon; Temecula, Calif.).

Immunohistochemistry. Immunohistochemistry was performed on fresh frozen slices of hippocampus from naïve 3-month-old Sprague-Dawley rats. Fresh-frozen slices of hippocampus were prepared by freezing dissected rat hippocampus in optimal cutting temperature compound (Sakura, Tokyo, Japan). The frozen hippocampus was then sectioned at 20 μm on superfrost-coated slides. Tissue slices were thawed and fixed with 4% sucrose/4% paraformaldehyde in PBS for 20 min at room temperature (RT). After washing with PBS, the sections were permeablized using 0.2% Triton X-100 in PBS and then blocked using 10% normal goat serum in PBS. The sections were again washed with PBS, then incubated at 4° C. overnight with affinity-purified anti-NPR Ab 4450 at a 1:200 dilution in blocking solution; control sections were also incubated with rabbit preimmune antibody and with Ab 4450 that had been pre-incubated with immunization antigen. The sections were washed and incubated 1 hr at RT with rhodamine-conjugated AffiniPure donkey anti-rabbit IgG antibody at a dilution of 1:500 in blocking solution (Jackson ImmunoResearch, West Grove, Pa.). After washing with PBS, the sections were cover slipped using the Prolong Antifade Kit (Molecular Probes, Eugene, Oreg.). Images were viewed and captured using a Nikon E800 epifluorescence microscope fitted with a Princeton Instruments CCD camera.

Immunocytochemical staining. Immunocytochemical staining of heterologous cells was performed as described (Xu et al., supra). For PMA-stimulation experiments, PMA (Sigma Cat #: P8139) at (150 nM) was added 90 minutes prior to live staining. Images were viewed and captured using Nikon E800 epifluorescence microscope fitted with a Princeton Instruments CCD camera. For live staining, neurons cultured on coverslips were incubated at 10° C. with the appropriate primary antibodies at the indicated dilutions in conditioned culture medium for 20 min. They were then washed with PBS and fixed with 4% paraformaldehyde/4% sucrose in PBS for 20 min at room temperature. Cells were permeablized with 0.2% Triton X-100 in PBS for 5 min and then blocked in 10% normal goat serum in PBS for 1 hr at room temperature. If intracellular proteins were of interest, coverslips were then incubated with antibody diluted in 10% normal goat serum for 1 hr at room temperature or overnight at 4° C. Coverslips were washed with PBS and incubated with secondary antibody conjugated to Alexa488 and Alexa555 (1:500 in 10% normal goat serum, Molecular Probes Eugene, Oreg.). Coverslips were mounted using the Prolong Antifade Kit (Molecular Probes, Eugene, Oreg.). For immunocytochemistry studies, mGluR1/5 were activated using the selective agonist (S)-DHPG (Tocris Cat.# 0805) with mGluR5 allosteric potentiator DFB (Tocris Cat.#1625). Neurons were incubated with DFB (50 μM) for 5 min and then DHPG (50 μM) for an additional 5 min in neuronal incubation medium and then transferred into the original incubation medium. In cases where TAPI-2 was used, TAPI-2 (50 μM, Calbiochem, La Jolla, Calif.) was preincubated with neurons 30 min before DHPG stimulations; neurons were incubated with TAPI-2 (50 μM) after DHPG stimulation as well. Cells were incubated at 37° C. to allow endocytosis. At indicated time points, cells were incubated with N-terminal GluR1 mouse antibody (JH 4093; 0.2 μg/100 μl; kind gift from T. Huganir) in neuronal incubation media (NIM) (1×MEM (Invitrogen), 2% Glutamax 1 (Invitrogen), 15 mM Hepes (Invitrogen), 1 mM sodium pyruvate (Invitrogen), 33 mM Glucose, 2% B27 (Invitrogen) in H₂O; pH 7.4) for 20 min at 10° C. (to block endocytosis during staining). Cells were then processed for immunofluorescent staining as described above. An acid strip protocol was employed to measure endocytosed antibody labeled GluR1 (Snyder et al., 2001). Neurons were washed with progressively cooler NIM to slow basal endocytosis. Cells were then live labeled at 10° C. with GluR1 N-terminal antibody (0.2 μg/100 μL JH4094) antibody in NIM for 20 min. After washing unbound excess antibody, cells were incubated sequentially with DFB, DHPG, and original incubation medium at 37° C., as described above. After appropriate incubation times, cells were washed with 4° C. TBS. Remaining surface bound GluR1 antibody was stripped with stripping buffer (0.5M NaCl, 0.2 M Acetic acid, pH 3.5) for 4 min on ice. Neurons were then washed with 4° C. TBS followed by processing for immunofluorescent staining as described above. All neurons were viewed and captured using UltraVIEW spinning disk confocal microscope (PerkinElmer Life Sciences, Wellesley, Mass., USA) with a Nikon Eclipse TE 200 microscope and a 63× objective (Nikon, Melville, N.Y.). Images were acquired and saved as Tiff files with a dynamic range of 65536 grey levels (16-bit binary). Image analysis was performed using Metamorph imaging software (Universal Imaging, Downingtown, Pa.). For quantification of the punctate structures (e.g. GluR1), images were thresholded by grey value to differentiate single puncta. Segments of dendrites were selected and number of puncta was calculated per 10 μL of length dendrite. Figures show ratios of calculated mean densities (number of puncta per unit length) from treated/control. Error bars were calculated using SEM and Gaussian error propagation. n=number of measured dendrites. Significance was determined by paired Student's T-test.

Identification of cleavage sites. Culture medium was harvested from 5×10 cm dish cultures of HEK293T cells transfected with myc-tagged NPR. The medium was incubated with mouse anti-myc monoclonal antibody 9e10 (Roche, Indianapolis, Ind.) covalently conjugated to 150 μL of GammaBind G Sepharose beads (Amersham Biosciences, Piscataway, N.J.) overnight at 4° C. with rotation. The beads were washed twice with 1% Triton X-100 and twice with PBS. Loading buffer (125 mM Tris (pH 6.8), with 4% SDS, 20% glycerol, and 0.016% bromophenol blue) was added to the beads and boiled for 2 min. The myc purified protein was then subjected to SDS-PAGE and transferred to Problott (Perkin-Elmer, Wellesly, Mass.). Purification of the proteins was confirmed by staining with Coomassie blue. The stained bands were cut out and subjected to automated Edman degradation at the Biosynthesis and Sequencing Facility, Johns Hopkins University School of Medicine (Dept. of Biological Chemistry), Baltimore, Md.

Cleavage Assay HEK293T cells were plated in OptiMEM (Invitrogen, Carlsbad, Calif.) containing 5% FBS onto 6-well dishes at a density of 4×10⁵ cells/mL (2 mL/well) 1 day before transfection. Cells were transfected with NPR in pRK5 using LipofectAMINE 2000 (Invitrogen, Carlsbad Calif.). After 24 hr the medium was replaced with serum-free OptiMEM. At 48 hr after transfection, a sample of medium was removed and the medium was replaced with serum-free OptiMEM containing the appropriate drug or vehicle: 150 nM PMA (Sigma Cat #: P8139), 25 μM GM6001 (Calbiochem Cat. # 444255), 25 μM GM6001 negative control (Calbiochem Cat. #364205), or 50 μM TAPI-2 (Calbiochem Cat. # 579052). Samples from each well were obtained at specific time points, immediately placed on ice, centrifuged at 15,000 g to remove cell debris, and then treated with 2% β-mercaptoethanol containing loading buffer. At the end of the time course, the cells were lysed with RIPA buffer, centrifuged to remove cell debris, and treated with 2% β-mercaptoethanol in loading buffer. Samples (20 μL of medium or lysate diluted to 1/5) were run on SDS-PAGE gels and analyzed by Western blotting.

Cell Surface Biotinylation. Surface biotinylation of GluR1 at the plasma membrane was described previously (Snyder et al., Nat Neurosci 4:1079-85, 2001) with some modifications. Cultured cortical neurons were treated with DHPG for 5 minutes. Medium was then replaced with previously cultured medium. After 45 minutes, neurons were cooled on ice and washed with cold PBS containing 1 mM CaCl₂ and 0.5 mM MgCl₂ (PBS++). Cell surface proteins were labeled with cell-impermeable EZ-Link Sulfo-NHS-SS-Biotin (sulfosuccinimidyl-2-(biotinamido)ethyl-1,3-dithiopropionate; Pierce) in PBS⁺⁺ (1 mg/mL) at 4° C. for 30 min. Unreacted biotin was quenched by washing cells with cold 100 mM Glycine (pH 7.4). Cultures were lysed using RIPA buffer and homogenates were centrifuged at 13, 200 rpm to remove debris. 10% of the homogenates were used for total protein. 90% of the remaining homogenates were incubated with immobilized NeutrAvidin beads (Pierce). After washing with RIPA buffer, bound proteins were eluted with 2× Laemmli sample buffer supplemented with 2% β-ME. The eluted proteins were subjected to SDS-PAGE and Western blotting followed by ECL (Amersham Biosciences) and exposure to autoradiography film. Primary antibody concentrations were as follows: GluR1N at 0.12 μg/mL and β-actin at 1:1000. Quantification performed on digital images scanned from exposure matched autoradiographs. Densitometric analysis of these digital images was performed using ImageJ software (Version 1.33, National Institutes of Health). Significance was determined by paired Student's T-test.

mGluR1/5-dependent LTD in hippocampus. Hippocampal slices from postnatal day 21 to 28 mice were prepared as previously described (Huber et al., Science 288:1254-72000). In brief, the brain was rapidly removed and placed in ice-cold modified artificial cerebrospinal fluid (aCSF; bubbled with 95% O₂/5% CO₂) which comprised: 110 mM choline chloride, 2.5 mM KCl, 7 mM MgCl₂, 0.5 mM CaCl₂, 2.4 mM sodium pyruvate, 1.3 mM sodium L-ascorbate, 1.2 mM NaH₂PO₄, 25 mM NaHCO₃, and 20 mM D-glucose. Transverse slices (400 μm thick) were cut on a Vibratome 3000 (The Vibratome company). Slices were transferred to a reservoir chamber filled with a normal aCSF which comprised (in mM): 124 mM NaCl, 2.5 mM KCl, 1.3 mM MgCl₂, 2.5 mM CaCl₂, 1 mM NaH₂PO₄, 26.2 mM NaHCO₃, and 20 D-glucose. Slices were allowed to recover for 2-6 hr at room temperature (22-24° C.). For recording, slices were transferred to an interface type chamber, maintained at 32° C. for 1 hr, and perfused continuously with aCSF at a rate of 2.5 mL/min. Standard methods were used to record field EPSPs (fEPSPs) from the stratum radiatum in response to stimulation of the Schaffer collateral-commissural pathway with a concentric bipolar tungsten electrode (FHC, Bowdoin, Me.). The stimulus intensity was adjusted to elicit 50-60% of the maximal response. Evoked responses were stored on-line and analyzed off-line using Clampfit (Ver. 9.2). LTD was induced using either by application of mGluR1/5-selective agonist 3,5-dihydroxyphenylglycine (DHPG; 50 μM, 5 min) or by a paired pulse low frequency stimulation protocol (PP-1 Hz: 50 ms interstimulus interval at 1 Hz for 15 min). The strength of the synaptic response was measured as the fEPFP slope. To study the characteristics of the basal synaptic transmission, either interstimulus intervals ranging from 30 to 150 msec for the measurement of paired-pulse facilitation or varied stimulus intensity to plot the synaptic responses against presynaptic fiber volley amplitude for the input-output relation was applied. LTD was expressed as percentage of the fEPSP slope during the baseline recording. All data shown for the time course are mean±SEM.

Recording and Ca²⁺ imaging in cerebellar cultures. Cultures of embryonic mouse cerebellum were prepared as previously described and recordings were performed according to our previously published method (Leitges et al., Neuron 44:585-94, 2004). Briefly, patch electrodes were filled with a solution containing: 135 mM CsCl, 10 mM HEPES, 0.5 mM EGTA, 4 mM Na2-ATP, and 0.4 mM Na-GTP, adjusted to pH 7.35 with CsOH. Cells were bathed in 140 mM NaCl, 5 mM KCl, 2 mM CaCl2, 0.8 mM MgCl2, 10 mM HEPES, 10 mM glucose, 0.0005 mM TTX, and 0.3 mM picrotoxin, adjusted to pH 7.35 with NaOH, which flowed at a rate of 0.5 mL/min. Patch electrodes were pulled from N51A glass and polished to yield a resistance of 2-4 MΩ. Iontophoresis electrodes (1 μm tip diameter) were filled with 10 mM glutamate (in 10 mM HEPES, pH 7.1) and were positioned ˜20 μm away from large-caliber dendrites. Test pulses were delivered using negative current pulses (300-700 nA, 30-80 msec duration). LTD-inducing pairing stimuli consisted of six, 3-s-long depolarizations to 0 mV, each delivered together with a test pulse of glutamate.

Membrane currents were recorded with an Axopatch 200A amplifier, and digitized at 10 kHz for exogenous glutamate responses and 20 kHz for mEPSCs. Signals were lowpass filtered at 5 kHz (1 kHz for mEPSCs) and acquired using Axograph X software (Axograph Scientific, Sydney, Australia). Recordings in which Rᵢₙₚᵤₜ or Rₛₑᵣᵢₑₛ varied by more than 10% were excluded from the analysis. For analysis of mEPSCs, Axograph X mini analysis software was used. This detected events based on closeness of fit of the mEPSC to a sliding template. Events smaller than − [symbol] 4 pA were discarded. A separate template was created for each recording by averaging 30 of its most unambiguous mEPSCs as selected by eye.

Bis-Fura-2 ratio imaging of intracellular free Ca²⁺ in the dendritic shafts of cultured Purkinje cells was accomplished by measuring the background corrected fluorescence ratio at 340 and 380 nm excitation using a cooled CCD camera system. In these experiments, EGTA was removed from the internal saline and replaced with 100 μM bis-Fura-2.

For gene delivery, a gold/glycerol mixture was first prepared as follows: 20 mg of 0.6 μm gold microcarriers were washed once in 70% ethanol and 3 times in sterile deionized, distilled water, and resuspended in 0.5 ml of sterile 50% glycerol. For each transfection into Purkinje cell cultures, 5 μg of plasmid cDNA, 1 μg of EGFP cDNA, 50 μL of 2.5 M CaCl₂, and 10 μL of 1 M spermidine were sequentially added to 50 μL of the gold/glycerol mixture. The preparation was gently vortexed for 15 minutes, after which the gold particles were centrifuged down and washed once with 70% ethanol and once with 100% ethanol. Washed gold particles were resuspended in 60 μL of 100% ethanol and pipetted onto macrocarrier disks (9 μL/disk). Following vortexing and an ethanol wash, microcarrier-containing solution was evaporated on the surface of six macrocarrier discs and delivered to the cerebellar cultures (at 6-7 DIV) using the Helios Gene Gun System (Bio-Rad) operating at a pressure of 450 psi and a vacuum of 20 in-Hg. Conditioned medium was aspirated from the surface of the cell culture immediately before transfection and was returned immediately thereafter. Cultures were then returned to the incubator for a minimum of 22 hours prior to electrophysiological recording. Transfected Purkinje cells were identified by imaging GFP signals with 480 nm illumination.

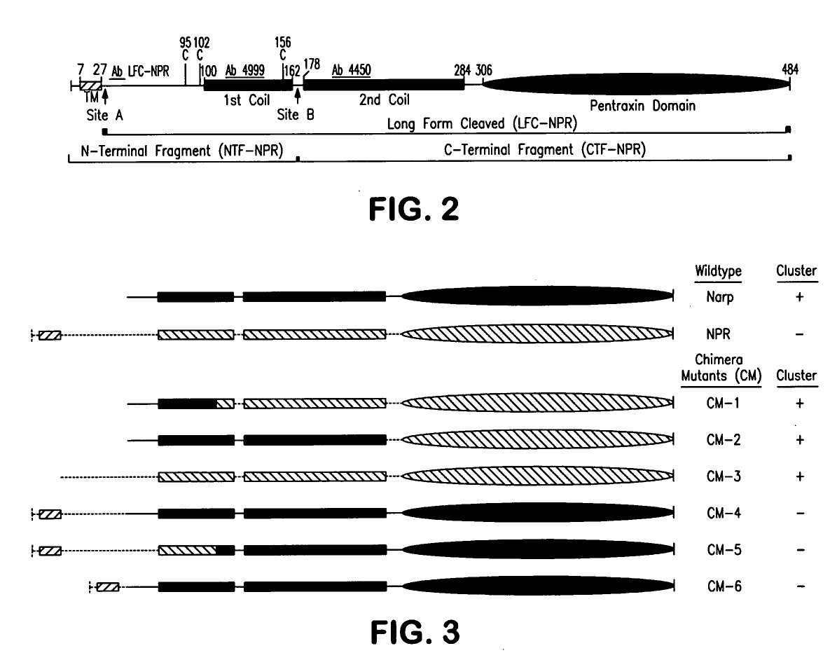

Full length NPR was expressed in HEK293T cells. Cell lysates and medium were assayed for expression of NPR using an antibody generated against a unique region in the 2nd coiled-coil domain (Ab 4450, FIG. 2). Consistent with its predicted molecular weight and presence of a transmembrane domain, NPR immunoreactivity was detected in cell lysates as a single band migrating at ˜62 kDa. When culture medium was examined using the same antibody (Ab 4450), bands at ˜62 kDa and ˜45 kDa were detected. The abundance of the lower molecular weight species in the medium suggested that NPR may be proteolytically processed. To examine this, the ˜62 kDa and ˜45 kDa bands were affinity purified from the medium and the amino acid sequence of each was determined by N-terminal Edman sequencing. The secreted ˜62-kDa species was identified to be a fragment of NPR that begins at Site A at amino acid L36, just C-terminal to the predicted transmembrane domain (FIG. 2). The ˜45 kDa species was identified as a NPR fragment that begins at Site B, at amino acid D176. Cleavage at Site B was predicted to produce a corresponding N-terminal fragment ending at amino acid A175 (FIG. 2). To test this, an additional antibody, Ab 4999, was generated against the first coiled-coil extracellular domain of NPR (FIG. 2). Ab 4999 detected a ˜62 kDa band in both the lysates and medium. Ab 4999 detected an additional band of ˜20 kDa present in the lysates, but not in the medium. This corresponds to the predicted molecular weight of a membrane associated N-terminal fragment produced by cleavage at Site B.

These data suggested that NPR undergoes cleavage at Site A (L36) and B (D176) to generate the fragments identified and illustrated in FIG. 2. The possibility that these products might arise from alternative splice forms of NPR can be excluded since the indicated fragments were isolated from transgene expression of cloned NPR cDNA. Cleavage at Site A (amino acid L36) produced a secreted product, designated “long form cleaved NPR” (LFC-NPR), which lacked the transmembrane domain and the short cytosolic region. LFC-NPR migrated on SDS-PAGE with nearly the same apparent molecular mass as full-length NPR, although on optimal gels, they resolved as a doublet. An antibody generated against the cytosolic N-terminus was not specific and was unable to detect the predicted residual N-terminus. Cleavage at Site B (amino acid D176) produced two cleavage products, designated “N-terminal fragment” (NTF-NPR) and “C-terminal fragment” (CTF-NPR). It is noteworthy that CTF-NPR was detected in the medium, whereas NTF-NPR was present only in lysates of transfected cells. This suggested that cleavage at Site B produced NTF-NPR that remains on the cell surface. Taken together, these data suggested that NPR was cleaved at either Site A or Site B, but not both, since a truncated NTF-NPR fragment was not detected in the medium.

Extracts from adult rat hippocampus, cerebellum, and cortex were analyzed for NPR cleavage products by Western blot. Ab 4450 and Ab 4999 detected bands that run at the expected ˜62 kDa after treatment with reducing reagent. Both antibodies detected the lower molecular weight species from the brain extracts: Ab 4450 detected a band of ˜45 kDa that corresponds to CTF-NPR, and Ab 4999 detected a band of ˜20 kDa that corresponds to NTF-NPR. All immunoreactivity for NPR using Ab 4450 or Ab 4999 was absent in brain extracts from mice that lack NPR.

Narp and NP1 form highly organized homo- and hetero-multimer complexes that are covalently linked by N-terminal disulfide bonds (Xu et al., supra). Moreover, NPR and Narp formed heteromultimer complexes when coexpressed in HEK293T cells. Heteromultimer complex formation is dependent on the first and third cysteines of NPs that flank the first coiled-coil domain (Xu et al., supra). NPR was co-expressed with mutants of Narp, in which two or all three of the cysteines are mutated to serine. In mutant “Narp (C1, 2S),” the cysteines at positions 14 and 79 were mutated to serines, and in mutant “Narp (C1, 2, 3S)” the cysteines at positions 14, 26, and 79 were mutated to serines. NPR did not co-immunoprecipitate (co-IP) with the mutant Narps. Thus, it appeared that NPR and Narp formed heterocomplexes that are stabilized by disulfide bonds. To examine NP complexes in brain, lysates from a control mouse were prepared and immunoprecipitated with rabbit antibody specific to NP1 or Narp. Lysates from knockout mice NP1 KO, Narp KO and combined NP1/Narp/NPR KO (NP TKO) were also utilized as controls. NPR (Ab 4999) specifically co-IPed with Narp and NP1. The ˜25 kDa band, presumptive NTF-NPR, also specifically co-IPed with both Narp and NP1. These data confirmed the specific association of NPR with Narp and NP1, and support the model that the N-terminal half of NPR mediates its interaction with NPs. The pentraxin domain of Narp and NP1 is required for interaction with AMPAR.

To determine if NPR would interact with AMPAR, AMPAR subunits from GluR1 or GluR6 were HA tagged (GluR1-HA and GluR6-HA, respectively) were co-transfected with myc-tagged Narp (Narp-myc) or NPR(NPR-myc) into HEK293T cells. Lysates were assayed for co-immunoprecipitation. GluR1-HA co-IPed with Narp-myc while GluR6-HA did not. Similarly, GluR1-HA but not GluR6-HA co-IPed with NPR-myc.

Analysis of NP protein expression in brain revealed strikingly different developmental profiles. In the hippocampus, Narp and NPR show marked increases during the second postnatal week that parallel the developmental increase of GluR1. In contrast, NP1 expression is highest early in development and decreases in adolescent and adult brain. It should be noted that even in the adult brain, NP1 protein is in stoichiometric excess to Narp by a ratio of >4:1.

Light microscopy revealed distinct distributions for each of the NPs that overlap in specific regions. For example, NPR and NP1 co-localized to strata oriens and radiatum of CA fields, while all three were enriched in the dentate hilus and mossy fiber projection to CA3. Narp and NPR were present in the molecular layer of the dentate gyrus but showed distinct laminar patterns. NPs were expressed in discrete localizations in the cerebellum as well. All three NPs were present in cell bodies of the Purkinje cell layer, and both NP1 and NPR were predominant in the molecular layer while Narp was relatively enriched in the granule cell layer. Thus, in combination with biochemical data that indicates all three NPs form complexes, the differential anatomic expression patterns suggest that the precise composition of the NP complex may be highly variable.

Hippocampal cultures were examined to assess the subcellular distribution of NPR in neurons. NPR antibody (Ab 4450) detected large clusters of NPR that co-localized with PSD-95, a marker for excitatory synapses, but which did not co-localize with glutamic acid decarboxylase, a marker for inhibitory axons. The subcellular localization of NPR was further examined using immunoelectron microscopy. Labeling of NPR with antibody (Ab 4450) was concentrated in various tubulovesicular structures, probably mostly endosomes, in presynaptic terminals and postsynaptic spines (CA3 stratum lucidum). Labeling of NPR was also seen in the synaptic cleft as well as in dense-core vesicles in or adjacent to the presynaptic active zone and contacting the membrane. These studies confirm that NPR is present at excitatory synapses and suggest an association with dynamic vesicular structures.

The ability of NPR to form clusters of AMPAR when expressed in COS-7 cells was examined. Unexpectedly, NPR was uniformly distributed on the cell surface. To test the hypothesis that the transmembrane domain of NPR might restrict its ability to form clusters, chimeric mutants (CM) of Narp and NPR were generated in which the N-terminal and C-terminal ends were swapped (FIG. 3). Chimeras CM-1 and CM-2, which include various lengths of Narp N-terminus that replace the NPR transmembrane domain, formed clusters on the surface of transfected COS-7 cells stained live with anti-myc antibody. In contrast, chimeras CM-4 and CM-5, which maintain one or both coiled-coil domains of Narp coupled to the NPR transmembrane domain, did not. Moreover, CM-3, in which the transmembrane domain of NPR is replaced with the signal peptide of Narp, and CM-6, in which the signal peptide of Narp is replaced with the transmembrane domain of NPR, demonstrated that the transmembrane domain of NPR is necessary (CM-3) and sufficient (CM-6) to inhibit pentraxin cluster formation. Consistent with the observation that NPR co-IPs AMPARs, CM-3 co-clustered GluR2. Thus, cleavage of the transmembrane domain allows NPR to cluster, and co-cluster AMPAR.

Regulated matrix metalloproteases (MMPs) can cleave type I and II transmembrane proteins (Black, Int J Biochem Cell Biol 34:1-5, 2002; Blobel, Curr Opin Cell Biol 12:606-12, 2000; Brown et al., Cell 100:391-8, 2000). Accordingly, experiments were conducted to determine whether cleavage of NPR could be induced by signaling pathways that activate MMPs. Cultures of cells transfected with NPR were treated with phorbol myristate acetate (PMA; 150 nM), which is known to activate MMPs. Medium from the cultures was sampled at subsequent time points. PMA increased the accumulation of LFC-NPR in the medium. The ˜45 kDa CTF-NPR form was also present in the medium, but its stimulated accumulation was less than LFC-NPR.

Based on these observations, it was hypothesized that cleavage at Site A was mediated by TACE, since TACE is reported to mediate PKC stimulated cleavage of Type I and II proteins such as TNFα, TGFα, and APP in multiple cell types. Consistent with this hypothesis, PMA-stimulated cleavage at Site A (resulting in the release LFC-NPR into the culture medium) was inhibited by the drug TAPI-2 (50 μM), which specifically inhibits TACE (Arribas et al., J Biol Chem 11376-82, 1996). Stimulated secretion of LFC-NPR into the medium was also inhibited by the general MMP inhibitor GM6001 (25 μM). Together, these observations implicated TACE in regulated cleavage of NPR at Site A. Cleavage at Site B was partially inhibited by TACE inhibitor.

LFC-NPR is similar to chimera CM3, which formed clusters that co-cluster AMPAR. Accordingly, the properties of LFC-NPR produced after stimulated cleavage with PMA were examined. COS-7 cells expressing NPR-myc were stained live with anti-myc antibody after treatment with PMA (150 nM) or vehicle. Transfected COS-7 cells treated with vehicle exhibited a uniform NPR distribution, typical of a membrane protein. When cells were treated with PMA (90 min) to promote cleavage and stimulate release of LFC-NPR, large clusters of NPR were detected. To determine if induction of NPR clusters by PMA resulted in co-clusters with AMPAR, COS-7 cells co-transfected with NPR-myc and GluR2-HA without PMA treatment exhibited were examined for clustering of NPR and GluR2. Diffuse NPR and GluR2 staining was observed. In contrast, COS-7 cells treated with PMA exhibited co-clusters of NPR and GluR2. These observations support the hypothesis that NPR cleavage by TACE induces NPR to cluster, and to co-cluster AMPAR.

To evaluate the localization of endogenous LFC-NPR as well as the regulation of its production by Site A cleavage in neurons, a cleavage-site selective antibody against LFC-NPR (Ab LFC-NPR) was generated using a synthetic peptide that mimics the new epitope at the N-terminus of LFC-NPR created by cleavage (FIG. 2). When Ab LFC-NPR was used to blot C-terminal tagged NPR-myc expressed in HEK293T cells, it selectively detected a ˜60 kDa protein that was enriched in the medium relative to lysates, while the anti-myc detected NPR equally in both fractions. Ab LFC-NPR detected a protein that migrates at ˜60 kDa molecular weight that superimposes with LFC-NPR detected by Ab 4450 in brain lysates from control mouse forebrain, and that was absent from brain lysates prepared from NPR KO mouse. These observations confirmed that Ab LFC-NPR selectively detects LFC-NPR.

The possibility that LFC-NPR associates with AMPAR on the surface of neurons was next examined. Cultured hippocampal neurons were transfected with GluR1 tagged with N-terminal extracellular α-bungarotoxin (BTX)-binding site (BBS). Surface GluR1-BBS and native LFC-NPR were then visualized by live application of rhodamine-linked BTX and Ab LFC-NPR. Labeling was performed at 10° C. to limit internalization. Live staining revealed co-clusters of LFC-NPR and GluR1-BBS on the surface of cultured hippocampal neurons. These findings indicated that LFC-NPR co-localizes with surface AMPAR.

Whether LFC-NPR co-localizes with AMPAR after endocytosis was next assessed. Surface GluR1-BBS and native NPR were labeled live with rhodamine-BTX at 10° C., and cultures were returned to 37° C. and then treated with 50 μM (S)-3,5-dihydroxyphenylglycine (DHPG) for 5 min. Transient application of DHPG, a selective agonist for mGluR1/5 receptors, produces rapid endocytosis of AMPAR. DHPG-containing medium was replaced with fresh medium and the cultures incubated for an additional 45 min. Cultures were then cooled to 4° C. Surface rhodamine-BTX and Ab LFC-NPR were removed using stripping buffer as described (Snyder et al., Nat Neurosci 4:1079-85, 2001). Cells were then permeablized and treated with fluorophore-linked rabbit secondary antibody to visualize internalized LFC-NPR. Internalized puncta of LFC-NPR and GluR1BBS co-localized in discrete puncta within dendrites suggested that LFC-NPR and AMPAR co-localize in endosomes. Moreover, NPR co-localized with EEA1, a marker for early endosomes. Finally, internalized LFC-NPR punctae often co-localized with PSD-95, suggesting that the vesicles play a role in trafficking to or from the synapse.

Double immunogold labeling with Ab LFC-NPR and antibodies to AMPAR was performed to assess if these proteins co-localize in postsynaptic endosomes in vivo. Antibodies to GluR2 were used in place of a monoclonal antibody to GluR1 for double labeling. The Ab LFC-NPR immunoEM was similar to that of NPR (Ab 4450), with labeling in tubulovesicular and vesicular structures in presynaptic terminals and postsynaptic spines in the CA1 and CA3 regions of the hippocampus and in the Purkinje spines of the cerebellar cortex. Labeling was also associated with distinctive endosomal complexes in the dendrite adjacent to the spine synapse, and in other endosomal structures. In double-labeling for LFC-NPR and AMPAR, co-localization was evident in the postsynaptic membrane and endosomal structures.

Ab LFC-NPR was used to examine the possibility that mGluR1/5 might regulate cleavage of NPR. mGluR1/5 activates PKC via phospholipase C and the production of diacylglycerol (DAG) and inositol triphosphate (IP₃) that releases intracellular Ca²⁺. Cultured cortical neurons (˜DIV 14) were treated with 50 μM DHPG for 5 minutes then returned to control medium for intervals of 0 to 90 min. Ab LFC-NPR detected a ˜60 kDa band from lysates that increased in intensity (expressed as a percent control) over time after stimulation with DHPG. No specific band was detected in the medium by Ab LFC-NPR or other NPR Abs. Absence of LFC-NPR in the medium of neurons was consistent with the observation that both Narp and NP1 were absent from medium even though they are secreted proteins detected on the cell surface using live labeling methods. These data suggested that secreted (Narp and NP1) and soluble (LFC-NPR) NP proteins remained associated with neuronal membranes in a manner that is distinct from heterologous cells. Together with studies that use Ab LFC-NPR for live labeling of neurons and trafficking studies, it was concluded that the ˜60 kDa band detected by Ab LFC-NPR is LFC-NPR. 90 minutes after stimulation with DHPG, levels of LFC-NPR increased by 60.7%±2.5%. Pretreatment of cultured neurons with TAPI-2 blocked the DHPG-stimulated increase in LFC-NPR. These results support the hypothesis that NPR undergoes TACE cleavage in neurons that can be regulated by mGluR1/5. They also establish that there is a substantial basal level of LFC-NPR on the surface of cultured neurons.

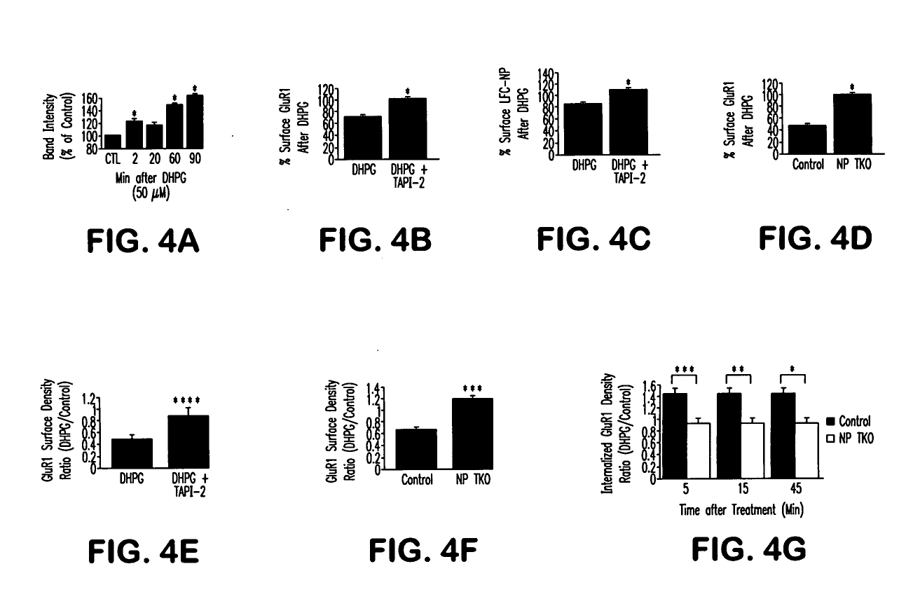

Activation of mGluR1/5 using DHPG in neurons produces a rapid and sustained reduction of surface AMPAR that represents a cellular model of LTD. Because NPR processing is induced by mGluR1/5 stimulation and LFC-NPR co-localizes with internalized AMPAR, the role that NPR processing might play in mGluR1/5-dependent trafficking of AMPAR was examined. Cell surface expression of GluR1 in cultured cortical neurons was assayed 45 min after DHPG treatment using the soluble biotinylating reagent to enrich surface proteins. Surface GluR1 was reduced 45 min after DHPG treatment compared to vehicle control treated cultured cortical neurons (74.1±2.2% of vehicle control). The effect of DHPG on surface GluR1 was blocked by pretreatment with TAPI-2 (102.4±4.9% of vehicle control) suggesting that TACE activity is necessary for DHPG mediated reduction of cell surface GluR1. Using the same technique, DHPG-mediated trafficking of NPR was next examined. Similar to GluR1, surface LFC-NPR was reduced after DHPG treatment (84.3±7.2% of vehicle control, FIG. 4C). These changes in LFC-NPR were blocked by pretreatment of cultures with TAPI-2 (108.9±16.0% of vehicle control).

To examine the role of NPs in mGluR1/5 mediated AMPAR surface expression, surface GluR1 in neurons cultured from control mouse and NP TKO mouse cortex after DHPG treatment were compared. Control neurons exhibited a significant reduction in surface GluR1 45 min after DHPG treatment (46.3±8.3% of vehicle control, FIG. 4D). In contrast, there was no change in surface GluR1 in NP TKO after treatment with DHPG (98.4±9.7% of vehicle control).

An immunocytochemical approach was employed to validate the biochemical findings. Cultured hippocampal neurons (DIV 14-17) were treated with DHPG for 5 min. In parallel studies, the addition of an mGluR5 allosteric potentiator (50 μM 3,3′-difluorobenzaldazine, DFB) 5 min before DHPG stimulation increased the consistency of the DHPG-evoked response. 45 min after DHPG stimulation, cells were progressively cooled to 10° C. and live labeled with GluR1 N-terminal specific antibody, fixed, and surface GluR1 detected via fluorescently labeled secondary antibodies. Consistent with the biochemical results, surface GluR1 puncta density was reduced in DHPG treated neurons (47.6±8.5% of vehicle control). There was little change in surface GluR1 puncta density after DHPG treatment if neurons were pretreated with TAPI-2 (84.7±12.6% of vehicle control). Surface GluR1 puncta density was also examined in cultured neurons from NP TKO and control mice. Again, surface GluR1 puncta density was reduced in DHPG treated neurons derived from control mice (68.5±6.0% of vehicle control) while surface GluR1 puncta density was unchanged by DHPG treatment in neurons cultured from NP TKO mice (113.4±6.3% of vehicle control). Therefore, the immunocytochemistry experiments confirmed the role of TACE activity and NP expression in mGluR1/5 mediated AMPAR cell surface reduction.

Internalization of AMPAR in neurons derived from NP TKO and control mice was examined using an acid-strip immunocytochemical staining protocol (Carroll et al., PNAS 96:14112-7, 1999). GluR1 Ab was added to cooled cultures and washed prior to the addition of DHPG, at defined times Ab on the cell surface was removed and internalized GluR1 assayed. DHPG treatment evoked an increase in internalized GluR1 in neurons derived from control mice (FIG. 4G). Internalized GluR1 was maximal within 5 min after addition of DHPG. This decreased at later time points (15 min and 45 min) presumably due to cycling of labeled GluR1 internalized through the internalized pool and reinsertion into the membrane. In contrast, DHPG treatment did not increase GluR1 internalization in neurons from NP TKO mice (FIG. 4G). The largest difference in DHPG induced GluR1 internalization density between control and NP TKO neurons was observed at the earliest time point (5 min), which suggested that NPs are required for mGluR1/5-dependent rapid endocytosis of GluR1.

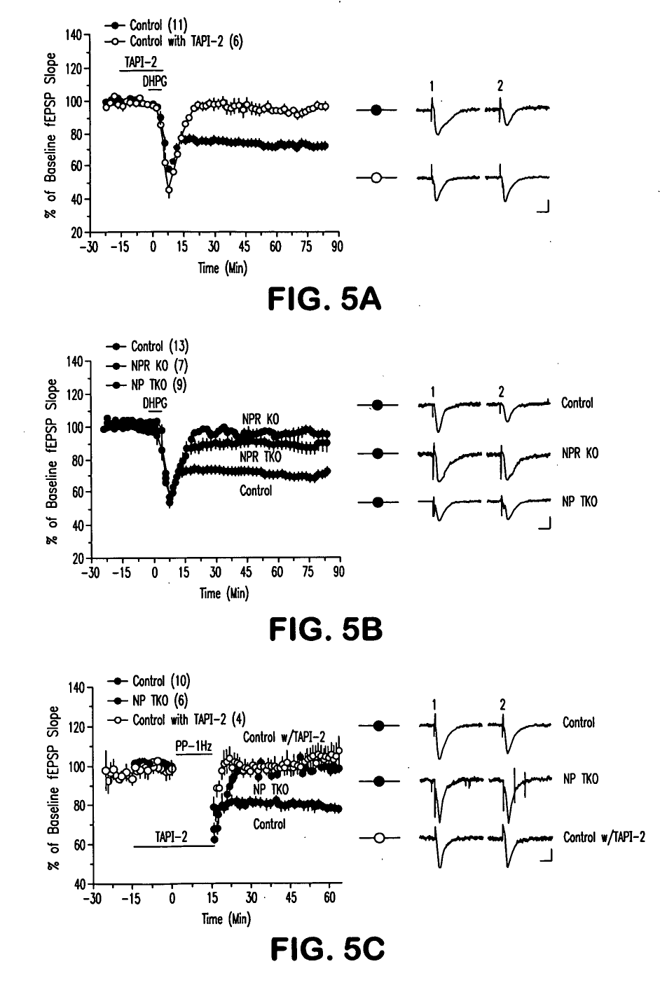

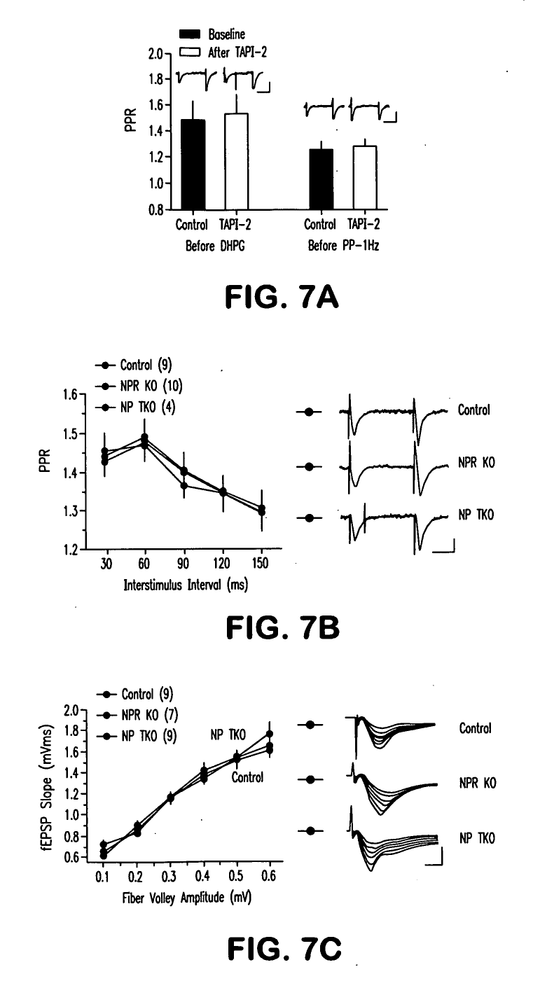

To evaluate the hypothesis that regulated cleavage of NPR plays a role in synaptic AMPAR trafficking, the Schaffer collateral-CA1 synapse was examined because it exhibits reliable mGluR1/5-dependent LTD that is mediated by postsynaptic AMPAR internalization (Camodeca et al., Neuropharmacology 38:1597-1606, 1999; Fitzjohn et al., Neuropharmacology 38:1577-83, 1999; Huber et al., J Neurophysiol 86:321-5, 2000; Kemp and Bashir, Neuropharmacology 38:495-504, 1999; Palmer et al., Neuropharmacology 36:1517-32, 1997; Snyder et al., Nat Neurosci 4:1079-85, 2001). Using field potential recording, a chemical form of mGluR1/5-dependent LTD in the CA1 region of control mouse (3-4 week old) hippocampal slices was examined. Application of the mGluR1/5 agonist DHPG (50 μM for 5 min) elicited a biphasic effect: a large depression of evoked field excitatory postsynaptic potential (fEPSP) slope during DHPG exposure, followed by a smaller depression of fEPSP slope that remains after washout (74.0±3.3% of baseline at t=75 min, n=11; FIG. 5A). Treatment of slices with TAPI-2 (50 μM for 20 min, starting 15 min before onset of DHPG) did not alter basal fEPSP slope or probability of release as indexed by paired pulse ratio (PPR) (FIG. 7A), and had no effect on the initial, phasic EPSP depression during DHPG treatment. However, TAPI-2 effectively blocked DHPG-induced LTD measured at t=75 min (93.8±2.5% of baseline, n=6; p<0.01; FIG. 5A). These results suggested that TACE activity is necessary for mGluR1/5-dependent hippocampal LTD.

Chemical mGluR1/5-dependent LTD in the hippocampal slices prepared from control, NPR KO, and NP TKO mice was next examined. Presynaptic function and basal synaptic transmission in hippocampal slices prepared from NPR KO and NP TKO at the Schaffer collateral-CA1 synapse exhibited no differences compared to control as indexed by PPR and fiber volley/fEPSP slope functions, respectively (FIGS. 7B and 7C). Moreover, the initial DHPG-induced depression of fEPSP slope was identical in control, NPR KO, and NP TKO slices (FIG. 5B). However, DHPG-induced LTD was significantly impaired in slices prepared from both NPR KO and NP TKO mice (96.3±2.5% n=7; p<0.01; 85.9±4.1% n=9; p<0.01, respectively) when compared to wild-type mice (68.6±3.7% n=13, FIG. 5B). A synaptically induced form of mGluR1/5-dependent LTD at the Schaffer collateral-CA1 synapse was next examined. Paired-pulse stimulation repeated at 1 Hz for 15 min (PP-1 Hz) at the Schaffer collateral-CA1 synapses produced an mGluR1/5 dependent form of LTD (Huber et al., Science 288:1254-7, 2000; Kemp and Bashir, supra). Using field recordings in control hippocampal slices, PP-1 Hz induced LTD (79.7±2.2%; n=10; FIG. 5C). Pretreatment of control slices with TAPI-2 (50 μM) blocked PP-1 Hz induced LTD (101.8±5.6% of baseline at t=50 min; n=4; p<0.01, FIG. 5C). Finally, PP-1 Hz induced LTD was absent in NP TKO slices (96.2±1.0%; n=6; p<0.01).

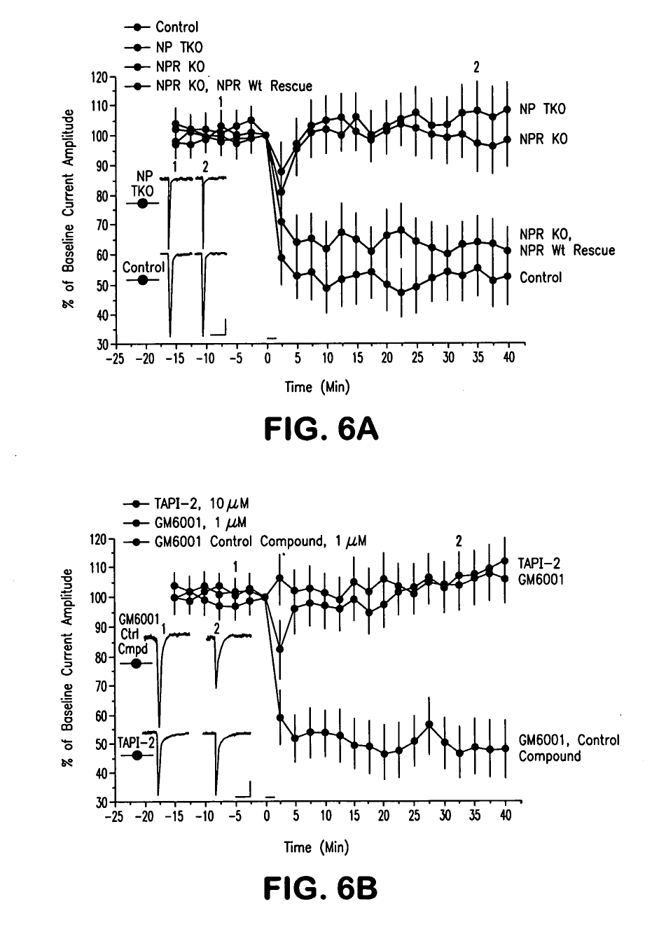

Other studies have shown that LTD in Purkinje cells cultured from embryonic mouse cerebellum requires activation of mGluR1, and is expressed postsynaptically as a reduction in surface AMPA receptors (Chung et al., Science 300:1751-5, 2003; Leitges et al., Neuron 44:585-94, 2004; Linden, PNAS 98:14066-71, 2001; Matsuda et al., EMBO J. 19:2765-74, 2000; Steinberg et al., Neuron 49:845-60, 2006; Wang and Linden, Neuron 25:635-47, 2000). Here, LTD was examined using a purely postsynaptic model in which iontophoretic test pulses of glutamate are applied to voltage-clamped Purkinje cells in culture. Following a baseline recording period, LTD was induced by pairing six 3 sec long depolarizing steps to 0 mV with six glutamate test pulses. When test pulses were resumed after pairing, LTD of the glutamate-evoked inward current was evident (52±9.0% of baseline at t=40 min, n=7 cells, FIG. 6A). When these experiments were repeated in cultures derived from NPR KO mice (98±8.6% of baseline, n=6) or NP TKO mice (108±9.3% of baseline, n=8), LTD induction was completely blocked (FIG. 6A). As a test of the specificity of the NPR KO blockade of LTD, a rescue experiment was performed. NPR KO Purkinje cells were subject to biolistic transfection with a plasmid driving strong expression of wtNPR. This manipulation succeeded in restoring LTD to near-control levels (61±7.6% of baseline, n=6, FIG. 6A).

To assess the role of TACE in cerebellar LTD, two different inhibitors were used: TAPI-2 (10 μM) and GM6001 (1 μM). Both of these treatments produced a complete blockade of LTD induction (112±8.6% and 106±8.4% of baseline at t=40 min, respectively, n=7 for both groups, FIG. 6B). However, application of the inactive GM6001 control compound (1 μM) failed to do so (48±9.8% of baseline, n=7, FIG. 6B). Importantly, none of these manipulations appeared to affect basal synaptic strength as indexed by mEPSC amplitude and kinetics (Table 2). Drugs and genetic manipulations can sometimes impact cerebellar LTD induction through their side effects on either mGluR1 function or voltage-sensitive Ca2+ channel function. None of the manipulations herein affected either depolarization-evoked or DHPG-evoked dendritic Ca2+ transients (Table 2), suggesting that these induction side effects cannot explain the observed blockade of LTD.

TABLE 2
Basal electrophysiological properties in cultured cerebellar Purkinje cells

| | | Resting | Depol- | DHPG- | | 10-90% | |
|---|---|---|---|---|---|---|---|
| | Rinput | Ca2+ | evoked | evoked | mEPSC | rise | 50% decay |
| Treatment | (Ω) | (nM) | (nM) | (nM) | amp (pA) | (msec) | (msec) |
| Control | 205 ± 30 | 115 ± 23 | 530 ± 67 | 245 ± 34 | 27 ± 7 | 2.0 ± 0.3 | 7.8 ± 0.5 |
| NP TKO | 188 ± 27 | 101 ± 26 | 557 ± 54 | 276 ± 38 | 34 ± 6 | 1.7 ± 0.3 | 8.5 ± 0.6 |
| NPR KO | 199 ± 25 | 96 ± 20 | 560 ± 60 | 259 ± 38 | 35 ± 7 | 1.6 ± 0.2 | 7.3 ± 0.5 |
| NPR KO, | 217 ± 32 | 95 ± 20 | 483 ± 49 | 280 ± 40 | 26 ± 5 | 1.7 ± 0.2 | 8.9 ± 0.7 |
| NPR wt | | | | | | | |
| rescue | | | | | | | |
| TAPI−2, | 202 ± 28 | 109 ± 22 | 604 ± 89 | 231 ± 35 | 27 ± 7 | 1.8 ± 0.4 | 7.7 ± 0.6 |
| 10 μM | | | | | | | |
| GM6001, | 210 ± 26 | 123 ± 27 | 557 ± 65 | 268 ± 39 | 25 ± 6 | 2.0 ± 0.3 | 8.0 ± 0.6 |
| 1 μM | | | | | | | |
| GM6001 | 196 ± 29 | 100 ± 25 | 526 ± 55 | 277 ± 42 | 26 ± 6 | 1.6 ± 0.3 | 7.6 ± 0.7 |
| control, | | | | | | | |
| 1 μM | | | | | | | |

Values are mean ± SEM. n = 10 Purkinje cells/treatment group.

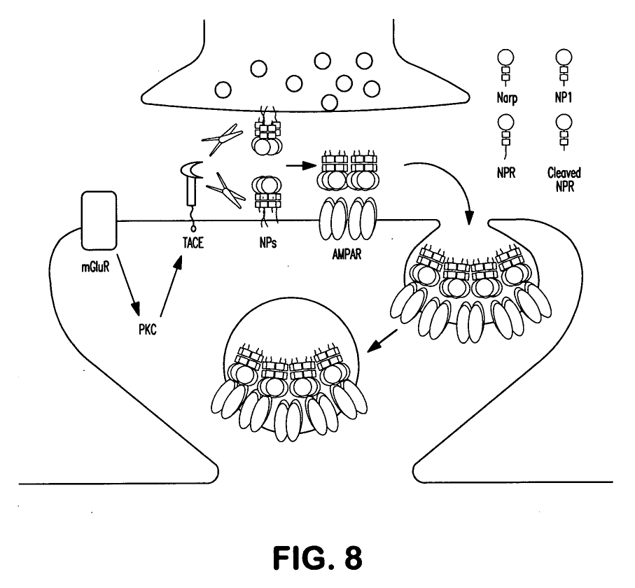

While not wishing to be held to a particular theory, the present findings suggest that NPR, and its regulated cleavage by TACE, are essential for group 1 mGluR1/5-dependent LTD (FIG. 8). mGluR1/5 induces the cleavage of NPR, which is blocked by selective TACE inhibitors. Cleaved NPR undergoes rapid internalization and co-localizes with AMPAR in postsynaptic vesicular structures that are confirmed by immunoEM. mGluR1/5 stimulation results in accelerated AMPAR endocytosis and a reduction of steady state AMPAR levels, and both of these processes are blocked by TACE inhibitors, and are absent in NP TKO neurons. Finally, mGluR1/5-dependent LTD is blocked by TACE inhibitors and is absent or dramatically reduced in neurons derived from mice lacking NPR. This model envisions that the transmembrane domain of NPR prevents its being incorporated into endosomes. Cleavage of NPR allows it, together with linked NPs and their associated pool of AMPAR, to enter endosomes, and thereby increases the efficacy of AMPAR endocytosis. This mechanism appears to be broadly relevant at excitatory synapses since similar responses are evident at both the hippocampal Schaffer collateral-CA1 synapse and in Purkinje cells in primary culture.

While not wishing to be held to a particular theory, the above model of mGluR-NPR function in synaptic plasticity implicates TACE, and perhaps other TACE-like metalloproteases, in a cellular process that has been described in other systems as ectodomain shedding. Extracellular metalloproteases have been implicated in the migration of neuronal precursor cells, axonal growth and guidance, neural cell adhesion, and activity dependent remodeling of neuronal connections. In particular, TACE is involved in the ectodomain shedding of membrane-anchored growth factors and cytokines such as the TNFα and the EGFR ligand TGFα. Ectodomain shedding of membrane proteins can release functional domains of a shed protein. For example, crosstalk between G-protein coupled receptor (GPCR) and EGF receptor (EGFR) involves metalloprotease-dependent release of pro-HB-EGF (Prenzel et al., Nature 402:884-8, 1999). In this process, GPCR activates a metalloprotease leading to the cellular release of membrane bound HB-EGF and activation of EGFR signaling via the released domain of HB-EGF. TACE-like proteases have been shown to act similarly on cell adhesion molecules. ADAM10 has been shown to cleave Ephrin-A5 when bound to its receptor, EphA3. Cleavage of Eprhin leads to the endocytosis of the Ephrin/Eph complex, which may function to mediate axonal growth cone collapse. In the brain, TACE expression is enriched in the hippocampus, cerebellar cortex, and cortex. The relationship of proteases and paradigms of learning and memory such as LTP have recently been appreciated. The present studies suggest that mGluR1/5 regulates TACE activity. The mechanism of coupling may involve PKC. Importantly, cleavage of NPR and endocytosis are closely coupled events. This is inferred from the fact that neurons possess substantial levels of LFC-NPR on their surface prior to DHPG stimulation, yet removal of LFC-NPR and AMPAR from the cell surface requires the action of TACE. Thus, the transmembrane domain appears important for both inhibiting NPR endocytosis prior to TACE activation and for LFC-NPR endocytosis after cleavage.

The molecular mechanisms governing mGluR1/5-dependent LTD are perhaps best understood in Purkinje cells. mGluR1/5 receptor activation results in generation of diacyl glycerol and release of Ca²⁺ from intracellular stores and these signals combine with Ca²⁺ influx via voltage-sensitive channels to activate PKC. Of the various isoforms of PKC, the classical PKCα, which is activated by combined DAG and Ca²⁺, has been implicated by KO and RNAi studies as well as by a transgenic mouse model expressing a PKC inhibitor (isoform non-specific) selectively in Purkinje cells. One of the actions of PKCα is to phosphorylate GluR2 at serine 880, and this has the effect of reducing its binding to the scaffolding protein GRIP. In contrast, the binding of the BAR domain containing protein PICK1 is not altered by phosphorylation and consequently, there results an increase in PICK1 binding to GluR2. The physiological correlate of enhanced PICK1 binding is an increase in GluR2 endocytosis rate and a reduction in steady state level of synaptic AMPAR. Other studies have examined the consequence of interrupted PICK1 binding to GluR2 in transgenic mice report increased AMPAR at the lateral margins of spines and within endosomal structures within spines. These localizations are consistent with the notion that PICK1 is important for endosomal trafficking of AMPAR.

While not wishing to be held to a particular mechanism, in the hippocampus, mGluR1/5-dependent LTD of the Schaffer collateral-CA1 synapse shares several mechanistic similarities to mGluR1/5-dependent LTD in the Purkinje cells. However, there are also important differences. Schaffer collateral/CA1 synapse LTD induced by mGluR1/5 is mediated by a postsynaptic increase in the rate of AMPAR endocytosis. However, hippocampal mGluR1/5-dependent LTD is insensitive to PKC specific inhibitors and is sensitive to phosphatase inhibitors. Moreover, mGluR1/5-dependent LTD in the medial perforant path of the dentate gyrus is blocked by PKC inhibitors (Huang et al., Neurosci Lett 274:71-74, 1999).

Another striking feature of mGluR1/5-dependent LTD at the Schaffer collateral-CA1 synapse is that its maintenance, after as brief a time as 10 min, is dependent on de novo protein translation of preexisting mRNA. The identity of the translated proteins that are required for this process is currently unknown. However, Arc/Arg3.1 is a candidate for such a protein. Its mRNA is robustly induced and transported to dendrites where is may be locally translated at synaptic sites. Additionally, Arc/Arg3.1 interacts with proteins involved in endocytotic machinery and modulates trafficking of AMPAR. NMDA dependent LTD at the Schaffer collateral-CA1 synapse is reduced in the Arc/Arg3.1 KO. Currently, the role of Arc/Arg3.1 in mGluR1/5-dependent LTD is not known but may involve maintenance of mGluR1/5-dependent LTD expression which has been shown to be protein synthesis dependent (Snyder et al., 2001). Thus, while not wishing to be held to a particular mechanism, TACE dependent cleavage of NPR may mediate the early phase of mGluR1/5-dependent LTD and Arc/Arg3.1 may mediate the maintenance of LTD expression.

Narp and NP1 are involved in synapse formation. Moreover, a motif within the X-domain of AMPAR, termed the NP association region (NAR), has been identified which is necessary and sufficient to bind Narp and to target glutamate receptors to synapses. Interestingly, a NAR-like sequence was identified in E- and N-cadherins, and these proteins were confirmed to bind Narp. Accordingly, the targeting of NPs to developing synapses may involve physical interactions of NPs with E- and N-cadherins, which are present at sites of cell-cell contract at the earliest stage of synapse formation. In such a model, NPs then act to restrict the diffusion of AMPAR present within the plasma membrane and capture the AMPAR at emerging excitatory synapses. While the physiology of synapse formation and synaptic depression, as revealed in the present study, appear overtly reciprocal, the ability of NPs to bind and cluster AMPAR may be central to both. In synapse formation, NPs capture and cluster AMPAR at synapses, while in synaptic depression, NPs capture AMPAR at sites of regulated endocytosis. It is notable that present model of NPs in LTD is dependent on NPR, which shows a marked developmental increase during the 2nd and 3rd postnatal weeks in the hippocampus.

Although the invention has been described with reference to the above examples, it will be understood that modifications and variations are encompassed within the spirit and scope of the invention. Accordingly, the invention is limited only by the following claims.

What is claimed is:

1. An isolated fragment of neuronal pentraxin receptor (NPR).

2. The fragment of claim 1, wherein the fragment is capable of internalizing AMPA receptors, clustering AMPA receptors, or modulating metabotropic glutamate receptor (mGluR) dependent long term depression (LTD) of a synapse.

3. The fragment of claim 1, wherein the fragment is a fragment produced by cleavage of NPR at cleavage site A or cleavage site B.

4. The fragment of claim 1, wherein the fragment is at least 90% identical to a sequence selected from the group consisting of SEQ ID NO:5, SEQ ID NO:7, and SEQ ID NO:9.

5. An isolated polynucleotide encoding the fragment of claim 1.

6. An isolated peptide comprising a Narp association region of an AMPA receptor, wherein the peptide is capable of binding a long pentraxin or a cadherin protein.

7. The peptide of claim 6, having at least 90% identity to the sequence set forth in SEQ ID NO: 11.

8. The peptide of claim 6, comprising a sequence selected from the group consisting of SEQ ID NO: 11, SEQ ID NO:24, and SEQ ID NO:25.

9. An isolated polynucleotide encoding a peptide of claim 6.

10. An antibody that blocks binding of a neuronal pentraxin to AMPAR.

11. The antibody of claim 10, wherein the antibody binds and Narp association region of AMPAR.

12. A method of identifying a compound that modulates metabotropic glutamate receptor (mGluR) dependent internalization of AMPA receptors, the method comprising:
(a) incubating the compound and a post-synaptic cell expressing a neuronal pentraxin receptor (NPR) or a functional fragment thereof and an mGluR1/5 under conditions sufficient to allow the compound to interact with the cell;
(b) determining the effect of the compound on the internalization of AMPA receptors upon stimulation with DHPG; and
(c) comparing the mGluR-dependent internalization of AMPA receptors upon stimulation with DHPG in the presence of compound, to the mGluR-dependent internalization of AMPA receptors upon stimulation with DHPG in the absence of compound, wherein a difference in the mGluR-dependent internalization of AMPA receptors indicates that the compound modulates mGluR-dependent internalization of AMPA receptors.

13. The method of claim 12, wherein the post-synaptic cell is a host cell containing an expression vector comprising a polynucleotide encoding NPR or encoding a functional, a conservative variant thereof.

14. The method of claim 12, wherein the effect is inhibition of the internalization of AMPA receptors.

15. The method of claim 12, wherein the effect is stimulation of the internalization of AMPA receptors.

16. A method of identifying a compound that modulates metabotropic glutamate receptor (mGluR) dependent long term depression (LTD) of a synapse, said method comprising:
(a) incubating the compound and a post-synaptic cell expressing a neuronal pentraxin receptor (NPR) or a functional fragment thereof and an mGluR1/5 under conditions sufficient to allow the compound to interact with the cell;
(b) determining the effect of the compound on the mGluR-dependent LTD upon stimulation with DHPG; and
(c) comparing the mGluR-dependent LTD upon stimulation with DHPG in the presence of compound to the mGluR-dependent LTD upon stimulation with DHPG in the absence of compound, wherein a difference in the mGluR-dependent LTD indicates that the compound modulates mGluR-dependent LTD.

17. The method of claim 16, wherein the post-synaptic cell is a host cell containing an expression vector comprising a polynucleotide encoding NPR or encoding a functional, a conservative variant thereof.

18. The method of claim 16, wherein the effect is inhibition of mGluR-dependent LTD.

19. The method of claim 16, wherein the effect is stimulation of mGluR-dependent LTD.


SEQUENCE LISTING (35 sequences)

SEQ ID NO: 1 - DNA, 2562 bp, Rattus norvegicus
  CDS (128)..(1423)
tggtgctggcgtttccctgcttgcacgcggttccctcgagcgccgctccgaccgacgtagccggccgcgaaggcgcccagacggcaagccagcgacccatgctgaagtgagcgcccaggtcagcgagatgctggcgctgctgaccgccggcgtggcgctcgccgtggccgcgggacaagcccaggataacccgatacctggcagtcgcttcgtgtgcaccgcgctgccccccgaagcggcgcgcgccggctgcccgctgcccgcgatgcccatgcagggaggcgcgctgagccctgaggaggagctgcgagccgctgtgctgcactggcgcgagaccgtcgtgcagcagaaggagacgctgggcgctcagcgagaagccatccgagaactcaccagcaagctggcccgctgtgagggactagccggcggtaaggcgcgcggcacgggggccacgggcaaggacaccatgggcgacctgccgcgggacccgggccacgtcgtggagcagcttagccgctcgctgcagaccctcaaggaccgcttggagagcctcgagctccaactccacaccaacgcgtctaatgccgggctgccgagcgacttccgagaggtgctccagcggaggctgggggagctggagaggcagttgctacgcaaggtggccgagctggaagacgagaagtccctgctccacaatgagacctcggctcaccggcagaagacagagaacacactgaatgcactgctgcagagggtgactgagctggagagaggcaacagtgcattcaagtcaccagatgcattcaaagtgtccctccctctccgtacaaactacctatacggcaagatcaagaagacgttgcccgagctgtatgccttcaccatctgcctgtggctgcggtccagcgcctcgccaggcatcggcacgccattctcctacgctgtgcctgggcaagccaatgagattgtgctgatagagtggggtaacaatcccatagagctgcttatcaacgacaaggtcgcacagctgcccctgtttgtcagcgatggcaagtggcaccatatctgcatcacctggaccactcgagacggcatgtgggaagcattccaggacggggagaagctgggcaccggggagaacctggcaccctggcatcccatcaagccagggggtgtgctcatcctggggcaggagcaggacactgtgggaggcagatttgatgccacacaggccttcgttggagagcttagccagttcaacatatgggaccgtgtcctccgggcacaagagatcatcaacatcgccaactgctccacgaacatgcctggaaacatcatcccatgggtggacaacaatgtcgatgtgtttggaggggcttccaagtggcctgtggagacgtgcgaagagcgtctcctggacttgtagctaccttctccctgtcccagaggccaagagcgggctgttctggggagttcaaggcatctattcccgagttcaactaaaatctctggcctgagtaggaaagaaccagagcccctaaggcaggctgtgtggcctcctttgtcttaggctcctatgttcttactgctttgttctttggtgggaagtgaccgaagccctgggaagagtcctgagccacttcctgctggggtttctagtaaagtctgtgagcctctccacccctcctgtaaatgctagtgcaacccagccctgcctgtcattttggatccttagtgtctcgtgtgtgcttcccgtctgtcccctttgatggctgtgtggtcatcctaccggggtggcctgggtcccttgtgtgtgtagcacatccctgcttttgactgaacacagtgcacagaagctacccgcccctgaaacagggtctctccctcagtgtcatgtgcactctggtctctccctctgaggggactgcagctgctggagggccagctgcccagacagtccccagcatccccaaagcagaccctccgccatggagaaagtcccccacagcttccccaccctctgtccacctctcagaccccacgcttctaaggaccattgctgggttggctttcaaaagctgctgctctcatctggtgccaaaagttcatttgcagcttctacaccgttctgtgtggtttggggattgactttattcccccacaaaagaggaacagccattagaagccagcctcccctccttttgatgctcagcccactgtgaagagtgagcttgcttgtaagccacattggtttctgtgagcatctgactctcccccgtccagtattttccccggaactggagattcgagcctagttcggctgctacctgcttagtgactccaggctgcatcatgtatcataatttattttaaagacaaagtgattcagtggggaaatttataaagctataaatattatatattttatttttcatacatgtttaaagtgcggatccatggatgttccatttgtaggaccagcttgacgtgcccatcctgacattgtatgccacaagagctcttgtgatgatggaattttgattaaagtgcactggaagatga

SEQ ID NO: 2 - protein, 432 aa, Rattus norvegicus
MLALLTAGVALAVAAGQAQDNPIPGSRFVCTALPPEAARAGCPLPAMPMQGGALSPEEELRAAVLHWRETVVQQKETLGAQREAIRELTSKLARCEGLAGGKARGTGATGKDTMGDLPRDPGHVVEQLSRSLQTLKDRLESLELQLHTNASNAGLPSDFREVLQRRLGELERQLLRKVAELEDEKSLLHNETSAHRQKTENTLNALLQRVTELERGNSAFKSPDAFKVSLPLRTNYLYGKIKKTLPELYAFTICLWLRSSASPGIGTPFSYAVPGQANEIVLIEWGNNPIELLINDKVAQLPLFVSDGKWHHICITWTTRDGMWEAFQDGEKLGTGENLAPWHPIKPGGVLILGQEQDTVGGRFDATQAFVGELSQFNIWDRVLRAQEIINIANCSTNMPGNIIPWVDNNVDVFGGASKWPVETCEERLLDL

SEQ ID NO: 3 - protein, 500 aa, Homo sapiens
MKFLAVLLAAGMLAFLGAVICIIASVPLAASPARALPGGADNASVASGAAASPGPQRSLSALHGAGGSAGPPALPGAPAASAHPLPPGPLFSRFLCTPLAAACPSGAQQGDAAGAAPGEREELLLLQSTAEQLRQTALQQEARIRADQDTIRELTGKLGRCESGLPRGLQGAGPRRDTMADGPWDSPALILELEDAVRALRDRIDRLEQELPARVNLSAAPAPVSAVPTGLHSKMDQLEGQLLAQVLALEKERVALSHSSRRQRQEVEKELDVLQGRVAELEHGSSAYSPPDAFKISIPIRNNYMYARVRKALPELYAFTACMWLRSRSSGTGQGTPFSYSVPGQANEIVLLEAGHEPMELLINDKVAQLPLSLKDNGWHHICIAWTTRDGLWSAYQDGELQGSGENLAAWHPIKPHGILILGQEQDTLGGRFDATQAFVGDIAQFNLWDHALTPAQVLGIANCTAPLLGNVLPWEDKLVEAFGGATKAAFDVCKGRAKA

SEQ ID NO: 4 - DNA, 5814 bp, Homo sapiens
cggccgcggcgacagctccagctccggctccggctccggctccggctccggctcccgcgcctgccccgctcggcccagcgcgcccgggctccgcgccccgaccccgccgccgcgcctgccgggggcctcgggcgcccccgccgcccgcctcacgctgaagttcctggccgtgctgctggccgcgggcatgctggcgttcctcggtgccgtcatctgcatcatcgccagcgtgcccctggcggccagcccggcgcgggcgctgcccggcggcgccgacaatgcttcggtcgcctcgggcgccgccgcgtccccgggcccgcagcggagcctgagcgcgctgcacggcgcgggcggttcagccgggccccccgcgctgcccggggcacccgcggccagcgcgcacccgctgccgcccgggcccctgttcagccgcttcctgtgcacgccgctggctgctgcctgcccgtcgggggcccagcagggggacgcggcgggcgctgcgccgggcgagcgcgaagagctgctgctgctgcagagcacggccgagcagctgcgccagacggcgctgcagcaggaggcgcgcatccgcgccgaccaggacaccatccgtgagctcaccggcaagctgggccgctgcgagagcggcctgccgcgcggcctccagggcgccgggccccgccgcgacaccatggccgacgggccctgggactcgcctgcgctcattctggagctggaggacgccgtgcgcgccctgcgggaccgcatcgaccgcctggagcaggagcttccagcccgtgtgaacctctcagctgccccagccccagtctctgctgtgcccaccggcctacactccaagatggaccagctggaggggcagctgctggcccaggtgctggcactggagaaggagcgtgtggccctcagccacagcagccgccggcagaggcaggaagtggaaaaggagttggacgtcctgcagggtcgtgtggctgagctggagcacgggtcctcagcctacagtcctccagatgccttcaagatcagcatccccatccgtaacaactacatgtacgcccgcgtgcggaaggctctgcccgagctctacgcattcaccgcctgcatgtggctgcggtccaggtccagcggcaccggccagggcacccccttctcctactcagtgcccgggcaggccaacgagattgtactgctagaggcgggccatgagcccatggagctgctgatcaacgacaaggtggcccagctgcccctgagcctgaaggacaatggctggcaccacatctgcatcgcctggaccacaagggatggcctatggtctgcctaccaggacggggagctgcagggctccggtgagaacctggctgcctggcaccccatcaagcctcatgggatccttatcttgggccaggagcaggataccctgggtggccggtttgatgccacccaggcctttgtcggtgacattgcccagtttaacctgtgggaccacgccctgacaccagcccaggtcctgggcattgccaactgcactgcgccactgctgggcaacgtccttccctgggaagacaagttggtggaggcctttgggggtgcaacaaaggctgccttcgatgtctgcaaggggagggccaaggcatgaggggccacctcatccagggcccctcccttgcctgccactttggggacttgaggggggtcatattccctcctcagcctgcccacgcactggccttccctcctgccccactcctggctgtgcctcccatttcccctcacctgtacccacacctccagaatgccctgccctgcgagtgtgtcccctgtccccacctgagtggggaggagcgtctcaagtgaacagtgggagcctgcccacctggcactgcactggagttgtctcttaccccaccctccctgcccatcaactgtatctgatttcactaattttgacagcacccccagtagggtaggattgtgtatgagggggaccccactatctcagtggtgggggtggccgcccgcccccttgtcccccatgcaacaggcccagtggcttccccttcagggccacaacaggctgtagaaggggatgacgaggacatcagaggttagacttaccctcctccctctttccaccagctgccagtcaagggcagtgggatctcgatggagcctccccccccccccacccatgcctccctcttcctcctctttcctcctctctttgtgtgtagcggtttgaatgttggttccatgcctggcccagccccacctcagtctccaggacattcctttcccagctccagcctggagggaaggggacaaagaccccaggaggccaaagggctgcagtcaccccttgtgctcacccatagtgatggccactggtatagtcatcgctctccctccatgccaaggacaggacttggaccgcttcagcctgggctgggagcagccctaaggtagaggcctcatggcccaggagaccccacctctggcagagccacattacctaccctgtgcatggtcctggggcagcaaggaagaagctcagagggtggggagaagcatgaagcagtgagcagagcactgggtgagagggagaagaccttggttcctagccagccctgctaatgtgctgtgtggccttctgtaagtccctgccctctctgggcctggccttcctcattcgtgagctgaggccctcgctttggtcatttgctctccagattgggtgtgagcttctctgtgattccaggtggatatgtggggaaagctctggtgaccctgggcttcgcaggggtagatcccaggactcggcagtggatgggatgcagccagtcatgggttagggtcagcagagactcagagtccagggcaaggttcaaggcagactaacctcatgcatggattgtaaaaaaccagctccctttggatcaacccagcctggcacccttgcctgtctgagagtgtctcaaagggctgatggcttcctggtccccttgagtcatcaccagcttccccaagagagtgtcagaatcttaagagctgagaggccgggcacggtggctcacgcctgtaatcccagcactttgggaggctgagacaggcagatcacttgaggtcaggagttcgaagtcagcctggccaacgtggtgaaaccccatcttcactaaaaatacaaaacttagctggttaggtggtgcatgcctgtagtcccagctactcgggaggccgaggcagaagaatctcttgaactgaggaggtggaggttgcagtgagccgagatcacgccattgcactccagcctgggcaacagagcaagactccatctcaaaaaaataataataatcttaaagatgagaaaagccaccccatctggcaccacagctgcatcttgcttgtgagaaatggggaagagttcagggaggacacgtgacctgcacaggatcacagagcatggggcagagccaggactagagctcagggcatctgactccctcttcagtgttcttccccctccatgttgcctgcccctgaagacctttgagttcagtctacacctaagcaggtagacatccgcgaggtcagatgctttccaacatgacacctgaacatcttcctttatgcaacacccaaacatcttggcatccccaccccaggaagtgcggggaggaggttatgatccctgggcgcttcggcagaatggagagctgaggtgtccctcccctgctagtcacctaccaggtgtctgagcagctgcatgctccctggctcaagtgggcactgtaccttttgcctgcctttttgttccctatctccactccctgaggccacttagcctgagacatgatgcaagagctgcaggccggggggctcagtgccatggaagctactccaagttgcattgcctcccgcgcccagatcctgctttccatttcgagaacataaatagattgcccagcccctccagtacaatcccactggaagaaaaggcaatggcgggcttcagccagacctgctgagacctaggttgccacggtaacagccaaagacatcaacccaagtgctgggtcaagtgtctcatcatactggcactgttgctggggtgacggcagaattcagaacttcaatttcagtgacgccaagcttgatgtgtttctgttattgttttgaagaaggtagctcttgtggaggacttgggagaaggatggggtcttaggaaggaggtgacagcacttgcatggtcacttgagcccacacacacgctcaaccccaagtcctttatgctttgtcacagtgaagatgagacctctgacgtccaagccttgttcctgtgctgcatcacccactcagccttccaaagggaacaggaacaaatttccccagcaccactgtttgggtcccgcttttcctatcttctgctgcccctgagcacatccaagcagacagggaaagaggagtcagacatggcccagtcacatcctgagctgctcctggctgataaccacgatggagcccgtgtttgtcctgccatctggcactgcactgagtgtggcacaggcaccgtcctgttgatctcacaacacagttctaagttaggacgttcttggctccgttagacaggtgaggaaactggggcacagagaggtgatgtcatctgcctggtgtcaatcagctagcaagtgatggagcccagatttcaaaccaaagggggttacgtccaggggctgagttcccactcacctgtgtagagtgccatctgggcaccattgctccagacgtgttccgacccctttcccagcccacagggcttgaagtgaaggaacagaggcagggggtgggccagccccagggccagggtccccttggtgaagccgtgccagggggctcagctgcttcagggaatgtgtccctcccaccatgggccagagcttcagcccttctttagctcagctagagttcacaggagagccaaaaaagaaaaggaagctgagcatctcccgagtcctgggcagggaaggggagggaaattgctgcttctccaactcttgcttggggccaagccctgcaccagttgcttcccagctgttatctgccagatcttcccatcttgtggcatgtggtgcccccaccaacatcccaaggggaccaatccccttgccaccactttgcatcacctgggaccacagatttggacaggaagggctctgagaagaggccaaagccctcattttacagatgaggaagctgaagcccggggaggggagcgaccctcaaggccacccagctggacacgggagacttgagcccagccttctgactgcattcagccctctctaggacgcagcagcctctccccagcactgagtcccccctcctttgtgtgtcccagcacccttggcctgagtaaacttggaaaggggctccctcccagagaagggactactctcttcacccctttattccagctgcctgccaccccagacccccacctcccaccctgacccccgacccctgggtggggaaggggctcacatgggcccaggctgagtgtgagtgagcatgtcaagttgtctgacactgtgacattagtgcaccctactgacaacccctccccagccttgcccctttctcctctccctgttttgtacataaattgacatgagctgcaacatgtgtgcgtgtgtgtgcgtgtgtgtgtgtgtgtatgtgtgtgtgatctgtgtcatggttttgttacctttttgtttttgtaaacttgaatgttcaaaataaacatgctgtttactctga

SEQ ID NO: 5 - protein, 175 aa, Homo sapiens
MKFLAVLLAAGMLAFLGAVICIIASVPLAASPARALPGGADNASVASGAAASPGPQRSLSALHGAGGSAGPPALPGAPAASAHPLPPGPLFSRFLCTPLAAACPSGAQQGDAAGAAPGEREELLLLQSTAEQLRQTALQQEARIRADQDTIRELTGKLGRCESGLPRGLQGAGPR

SEQ ID NO: 6 - protein, 43 aa, Rat
VDWKRPKYTSALTYDGVKVMAEAFQSLRRQRIDISRRGNAGDC

SEQ ID NO: 7 - protein, 459 aa, Homo sapiens
LPGGADNASVASGAAASPGPQRSLSALHGAGGSAGPPALPGAPAASAHPLPPGPLFSRFLCTPLAAACPSGAQQGDAAGAAPGEREELLLLQSTAEQLRQTALQQEARIRADQDTIRELTGKLGRCESGLPRGLQGAGPRRDTMADGPWDSPALILELEDAVRALRDRIDRLEQELPARVNLSAAPAPVSAVPTGLHSKMDQLEGQLLAQVLALEKERVALSHSSRRQRQEVEKELDVLQGRVAELEHGSSAYSPPDAFKISIPIRNNYMYARVRKALPELYAFTACMWLRSRSSGTGQGTPFSYSVPGQANEIVLLEAGHEPMELLINDKVAQLPLSLKDNGWHHICIAWTTRDGLWSAYQDGELQGSGENLAAWHPIKPHGILILGQEQDTLGGRFDATQAFVGDIAQFNLWDHALTPAQVLGIANCTAPLLGNVLPWEDKLVEAFGGATKAAFDVC

SEQ ID NO: 8 - protein, 43 aa, Rat
AHTATIKYTSALTYDAVQVMTEAFRNLRKQRIEISRRGNAGDC

SEQ ID NO: 9 - protein, 319 aa, Homo sapiens
RDTMADGPWDSPALILELEDAVRALRDRIDRLEQELPARVNLSAAPAPVSAVPTGLHSKMDQLEGQLLAQVLALEKERVALSHSSRRQRQEVEKELDVLQGRVAELEHGSSAYSPPDAFKISIPIRNNYMYARVRKALPELYAFTACMWLRSRSSGTGQGTPFSYSVPGQANEIVLLEAGHEPMELLINDKVAQLPLSLKDNGWHHICIAWTTRDGLWSAYQDGELQGSGENLAAWHPIKPHGILILGQEQDTLGGRFDATQAFVGDIAQFNLWDHALTPAQVLGIANCTAPLLGNVLPWEDKLVEAFGGATKAAFDVC

SEQ ID NO: 10 - protein, 43 aa, Rat
AKNAPPKYTSALTHDAILVIAEAFRYLRRQRVDVSRRGSAGDC

SEQ ID NO: 11 - protein, 43 aa, Homo sapiens; identical to SEQ ID NO: 6

SEQ ID NO: 12 - protein, 43 aa, Rat
SETPPPKYTSALTYDGVLVMAETFRSLRRQKIDISRRGNAGDC

SEQ ID NO: 13 - protein, 432 aa, Homo sapiens
MPAGRAARTCALLALCLLGAGAQDFGPTRFICTSVPVDADMCAASVAAGGAEELRSSVLQLRETVLQQKETILSQKETIRELTAKLGRCESQSTLDPGAGEARAGGGRKQPGSGKNTMGDLSRTPAAETLSQLGQTLQSLKTRLENLEQYSRLNSSSQTNSLKDLLQSKIDELERQVLSRVNTLEEGKGGPRNDTEERVKIETALTSLHQRISELEKGQKDNRPGDKFQLTFPLRTNYMYAKVKKSLPEMYAFTVCMWLKSSATPGVGTPFSYAVPGQANELVLIEWGNNPMEILINDKVAKLPFVINDGKWHHICVTWTTRDGVWEAYQDGTQGGSGENLAPYHPIKPQGVLVLGQEQDTLGGGFDATQAFVGELAHFNIWDRKLTPGEVYNLATCSTKALSGNVIAWAESHIEIYGGATKWTFEACRQIN

SEQ ID NO: 14 - protein, 40 aa, Rat
KPDSGLLDGFMTIDAALMYDAVHVVSVAVQQFPQMTVSSL

SEQ ID NO: 15 - DNA, 30 bp, Artificial Sequence
  Primer
aaaaagaattcccggtaccggctcttgctc

SEQ ID NO: 16 - DNA, 31 bp, Artificial Sequence
  Primer
tctggtctacgagaaggagaagatctttttt

SEQ ID NO: 17 - DNA, 69 bp, Artificial Sequence
  Primer
cgttctcttcccgattccgttctagatacctcgttttcgagtaaagactccttctagagttaaagatct

SEQ ID NO: 18 - DNA, 72 bp, Artificial Sequence
  Primer
ctacacacgttctcttcccgattccgttctagatacataggtatactgcagggtctgatacgtatcagatct

SEQ ID NO: 19 - DNA, 25 bp, Artificial Sequence
  Primer
ggatcccgcgacactatggccgacg

SEQ ID NO: 20 - DNA, 25 bp, Artificial Sequence
  Primer
ggatgtcagggggtctacgcagctg

SEQ ID NO: 21 - DNA, 26 bp, Artificial Sequence
  Primer
ggatccctcagtcgcttcctctgcac

SEQ ID NO: 22 - DNA, 25 bp, Artificial Sequence
  Primer
ctgtggtaagcgctcgagcagctgt

SEQ ID NO: 23 - protein, 11 aa, Artificial Sequence
  Synthetic construct
LPPGTDNASAC

SEQ ID NO: 24 - protein, 12 aa, Homo sapiens
LRRQRIDISRRG

SEQ ID NO: 25 - protein, 45 aa, Homo sapiens
SVFVRTTEEGMIRVRKSKGKYAYLLESTMNEYIEQRKPCDTMKVG

SEQ ID NO: 26 - protein, 45 aa, Rat; identical to SEQ ID NO: 25

SEQ ID NO: 27 - protein, 45 aa, Rat
SVFVRTTAEGVARVRKSKGKYAYLLESTMNEYIEQRKPCDTMKVG

SEQ ID NO: 28 - protein, 45 aa, Rat
SVFTKTTADGVARVRKSKGKFAFLLESTMNEYIEQRKPCDTMKVG

SEQ ID NO: 29 - protein, 45 aa, Rat
SVFTRTTAEGVARVRKSKGKFAFLLESTMNEYIEQRKPCDTMKVG

SEQ ID NO: 30 - protein, 42 aa, Rat
SVLVKSNEEGIQRVLTSDYAFLMESTTIEFVTQRNCNLTQIG

SEQ ID NO: 31 - protein, 42 aa, Rat
DWKRPKYTSALTYDGVKVMAEAFQSLRRQRIDISRRGNAGDC

SEQ ID NO: 32 - protein, 42 aa, Mouse; identical to SEQ ID NO: 31

SEQ ID NO: 33 - protein, 42 aa, Homo sapiens; identical to SEQ ID NO: 31

SEQ ID NO: 34 - protein, 41 aa, Zebrafish
KRRLKYTGALTYDGVKVMSTAFQNLRRQRIDISRRGNAGEC

SEQ ID NO: 35 - protein, 42 aa, Artificial Sequence; identical to SEQ ID NO: 31
  Consensus sequence